SEQUENCE LISTING (48 sequences)

SEQ ID NO: 1 - protein, 119 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSS

SEQ ID NO: 2 - protein, 5 aa, Artificial Sequence
  Description of Artificial Sequence Synthetic peptide
DYGVH

SEQ ID NO: 3 - protein, 16 aa, Artificial Sequence
  Description of Artificial Sequence Synthetic peptide
VIWSGGGTAYNTALIS

SEQ ID NO: 4 - protein, 11 aa, Artificial Sequence
  Description of Artificial Sequence Synthetic peptide
RGSYPYNYFDA

SEQ ID NO: 5 - protein, 110 aa, Artificial Sequence
  Description of Artificial Sequence Synthetic polypeptide
QAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 6 - protein, 14 aa, Artificial Sequence
  Description of Artificial Sequence Synthetic peptide
GSSTGAVTASNYAN

SEQ ID NO: 7 - protein, 7 aa, Artificial Sequence
  Description of Artificial Sequence Synthetic peptide
GHNNRPP

SEQ ID NO: 8 - protein, 9 aa, Artificial Sequence
  Description of Artificial Sequence Synthetic peptide
ALWYSDHWV

SEQ ID NO: 9 - protein, 15 aa, Artificial Sequence
  Description of Artificial Sequence Synthetic peptide
GGGGSGGGGSGGGGS

SEQ ID NO: 10 - protein, 20 aa, Artificial Sequence
  Description of Artificial Sequence Synthetic peptide
GGGGSGGGGSGGGGSGGGGS

SEQ ID NO: 11 - protein, 30 aa, Artificial Sequence
  Description of Artificial Sequence Synthetic polypeptide
GGGGSGGGGSGGGGSGGGGSGGGGSGGGGS

SEQ ID NO: 12 - protein, 211 aa, Homo sapiens
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 211 aa, Homo sapiens
GPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 14 - protein, 211 aa, Homo sapiens
GPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 211 aa, Homo sapiens
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 233 aa, Homo sapiens
DLEPKSPDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 28 aa, Unknown
  Description of Unknown CD4 sequence
MALIVLGGVAGLLLFIGLGIFFCVRCRH

SEQ ID NO: 18 - protein, 71 aa, Unknown
  Description of Unknown CD8 sequence
PTTTPAPRPPTPAPTIASQPLSLRPEACRPAAGGAVHTRGLDFACDIYIWAPLAGTCGVLLLSLVITLYCN

SEQ ID NO: 19 - protein, 27 aa, Unknown
  Description of Unknown CD28 sequence
FWVLVVVGGVLACYSLLVTVAFIIFWV

SEQ ID NO: 20 - protein, 21 aa, Unknown
  Description of Unknown CD3zeta sequence
LCYLLDGILFIYGVILTALFL

SEQ ID NO: 21 - protein, 27 aa, Unknown
  Description of Unknown 4-1BB ligand receptor sequence
IISFFLALTSTALLFLLFFLTLRFSVV

SEQ ID NO: 22 - protein, 25 aa, Unknown
  Description of Unknown CD4 signal peptide sequence
MNRGVPFRHLLLVLQLALLPAATQG

SEQ ID NO: 23 - protein, 21 aa, Unknown
  Description of Unknown CD8 signal peptide sequence
MALPVTALLLPLALLLHAARP

SEQ ID NO: 24 - protein, 21 aa, Unknown
  Description of Unknown CD8 signal peptide sequence
MRPRLWLLLAAQLTVLHGNSV

SEQ ID NO: 25 - protein, 18 aa, Unknown
  Description of Unknown CD28 signal peptide sequence
MLRLLLALNLFPSIQVTG

SEQ ID NO: 26 - protein, 20 aa, Unknown
  Description of Unknown IL2 signal peptide sequence
MYRMQLLSCIALSLALVTNS

SEQ ID NO: 27 - protein, 245 aa, Artificial Sequence
  Description of Artificial Sequence Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 28 - protein, 263 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 29 - DNA, 744 bp, Artificial Sequence
  Description of Artificial Sequence Synthetic polynucleotide
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgca

SEQ ID NO: 30 - DNA, 789 bp, Artificial Sequence
  Description of Artificial Sequence Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 31 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 251 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

SEQ ID NO: 35 - protein, 42 aa, Homo sapiens
KRGRKKLLYIFKQPFMRPVQTTQEEDGCSCRFPEEEEGGCEL

SEQ ID NO: 36 - protein, 276 aa, Homo sapiens
MCVGARRLGRGPCAALLLLGLGLSTVTGLHCVGDTYPSNDRCCHECRPGNGMVSRCSRSQNTVCRPCGPGFYNDWSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 37 - protein, 196 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVWCILGCILICWLTKKKYSSSVHDPNGEYMFMRATAKKSRLTDVTL

SEQ ID NO: 38 - protein, 220 aa, Homo sapiens
MACLGFQRHKAQLNLATRTWPCTLLFFLLFIPVFCKAMHVAQPAWLASSRGIASFVCEYASPGKATEVRVTVLRQADSQVTEVCAATYMMGNELTFLDDSICTGTSSGNQLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 39 - protein, 286 aa, Homo sapiens
MQIPQAPWPVVWAVLQLGWRPGWFLDSPDRPWNPPTFSPALLWTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGWGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 40 - protein, 524 aa, Homo sapiens
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 41 - protein, 368 aa, Homo sapiens
MLGQWTLILLLLLKVYQGKGCQGSADHWSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 42 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 43 - protein, 777 aa, Artificial Sequence
  Description of Artificial Sequence Synthetic polypeptide
TMNRGVPFRHLLLVLQLALLPAATQGHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGDLEPKSPDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAAAMALIVLGGVAGLLLFIGLGIFFCVRCRHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 44 - protein, 491 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 1053 aa, Artificial Sequence
  Description of Artificial Sequence Synthetic polypeptide
TMNRGVPFRHLLLVLQLALLPAATQGHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGKDPKAAAMALIVLGGVAGLLLFIGLGIFFCVRCRHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKATNFSLLKQAGDVEENPGPALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 823 aa, Artificial Sequence
  Description of Artificial Sequence Synthetic polypeptide
TMNRGVPFRHLLLVLQLALLPAATQGHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGHHHHHHDKLVKCEGISLLAQNTSWLLLLLLSLSLLQATDFMSLATNFSLLKQAGDVEENPGPMALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 4 aa, Unknown
  Description of Unknown CTLA-4  motif sequence
YVKM

SEQ ID NO: 48 - protein, 6 aa, Artificial Sequence
  Description of Artificial Sequence Synthetic 6xHis tag
HHHHHH

(19) United States
(12) Patent Application Publication
(10) Pub. No.: US 2023/0346988 A1
(43) Pub. Date: Nov. 2, 2023

(54) SYSTEMS FOR IN VIVO MONITORING OF IMMUNE CELLS IN PATIENTS UNDERGOING CELLULAR IMMUNOTHERAPY

(71) Applicant: Memorial Sloan Kettering Cancer Center, New York, NY (US)

(72) Inventors: Steven Larson, New York, NY (US); Darren Veach, New York, NY (US); Sarah Cheal, New York, NY (US); Ouathek Ouerfelli, New York, NY (US); Guangbin Yang, New York, NY (US); Nai-Kong Cheung, New York, NY (US); Brian Santich, New York, NY (US); Simone Krebs, New York, NY (US); Megan Dacek, New York, NY (US); David Scheinberg, New York, NY (US)

(21) Appl. No.: 18/003,645

(22) PCT Filed: Jun. 28, 2021
(86) PCT No.: PCT/US2021/039420

§ 371 Date: Dec. 28, 2022

Related U.S. Application Data
(60) Provisional application No. 63/045,628, filed on Jun. 29, 2020.

(51) Int. Cl. A61K 51/04; C07K 16/28; C07K 16/44; A61K 39/00

(52) U.S. Cl. CPC A61K 51/0482; C07K 16/2803; A61K 51/0497; C07K 16/44; A61K 39/4637; C07K 2319/02; C07K 2319/03; A61K 2239/38; C07K 2317/622; C07K 2317/31; C07K 2319/33

(57) ABSTRACT

The present disclosure provides kits and methods for tracking or monitoring in vivo biodistribution, viability, and/or expansion of immune cells in a cancer patient undergoing cellular immunotherapy.

### CROSS-REFERENCE TO RELATED APPLICATIONS

This application is a U.S. National Stage Application of PCT/US2021/039420, filed Jun. 28, 2021, which claims the benefit of and priority to U.S. Provisional Patent Application No. 63/045,628, filed Jun. 29, 2020, the contents of which are incorporated by reference herein in its entirety.

### STATEMENT OF GOVERNMENT SUPPORT

This invention was made with government support under CA008748 and CA184746 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 13, 2021, is named 115872-2238_SL.txt and is 77,969 bytes in size.

### TECHNICAL FIELD

The present technology relates generally to kits and methods for tracking or monitoring in vivo biodistribution, viability, and/or expansion of immune cells in a cancer patient undergoing cellular immunotherapy.

### BACKGROUND

The following description of the background of the present technology is provided simply as an aid in understanding the present technology and is not admitted to describe or constitute prior art to the present technology.

CAR T cell therapy has been especially potent for tumors of the blood and lymph nodes, i.e., leukemia and lymphoma. However, many tumors do not respond well to CAR T cell therapy, especially solid tumors of the colon, lung, and breast. D'Aloia et al., Cell Death & Disease 9, 282 (2018). One reason might be that CAR-T cells do not find their way to the tumor due to a variety of resistance mechanisms, including the tumor microenvironment that deflects immune surveillance and attack. Other challenges include heterogeneously expressed tumor target antigens and impaired long-term persistence of CAR T cells at the tumor site.

Accordingly, there is an urgent need in tumor immunology for compositions and methods that “track” or “trace” CAR T cells within the body of a subject.

### SUMMARY OF THE PRESENT TECHNOLOGY

Provided herein are kits and methods for creating specialized reporter immune cells that are capable of providing real-time updates on the in vivo behavior/activity (e.g., in vivo biodistribution, viability, and/or expansion) of immune cells of any immune specificity in a subject suffering from cancer.

In one aspect, the kits may include non-endogenous expression vectors comprising recombinant nucleic acids encoding any of the fusion proteins disclosed herein, and instructions for transducing the non-endogenous expression vectors (e.g., viral vectors) into immune cells and tracking or monitoring in vivo biodistribution, viability, and/or expansion of the transduced recombinant immune cells. The immune cells may be derived from an autologous donor or an allogenic donor. Additionally or alternatively, the kits of the present technology further comprise a biotinylated DOTA-based hapten; and one or more DOTA-bearing bischelate(s). The biotinylated DOTA-based hapten is useful for FACS, quantification of DOTA binding sites in transfected immune cells, and histology assays. In some embodiments, the one or more DOTA-bearing bischelate(s) in the kit may be labelled with a suitable radionuclide.

Additionally or alternatively, in some embodiments, at least one of the non-endogenous expression vectors, the biotinylated DOTA-based hapten; and one or more DOTA-bearing bischelate(s) may be stored in lyophilized form. In any of the embodiments disclosed herein, the kits can also comprise, e.g., a buffering salts (e.g., for radiochemistry and/or reconstituting the lyophilized contents of the kit to generate isotonic solutions suitable for use in cell assays or ex vivo inoculation), excipients, radioprotectants, preservatives, stabilizing agents, cell culture medium, cell culture supplements, ITLC strips, and the like. Excipients that may aid in resuspension, stability and shelf life of the non-endogenous expression vectors (e.g., viral vectors), biotinylated DOTA-based hapten or DOTA-bearing bischelate(s) include ascorbic acid (as an antioxidant), sodium benzoate (e.g., 0.5% sodium benzoate as a preservative) and optionally stabilizers to preserve viral infectivity and titer such as sorbitol (e.g., 10% sorbitol). Examples of buffers that are compatible with radiochemistry are NH₄OAc (ammonium acetate), NaOAc, (sodium acetate), or HEPES (4-(2-hydroxyethyl)-1-piperazineethanesulfonic acid). The typical patient dose for radiolabeled DOTA-bearing bischelate(s) ranges from 5-10 mCi for imaging applications. The one or more DOTA-bearing bischelate(s) contained in the kit may be present in an amount sufficient for one or patient doses, as well as quality control testing by radio-instant TLC or radio-HPLC.

The kits of the present technology can further comprise components necessary for determining biotin-streptavidin binding competence of the biotinylated DOTA-based hapten, such as streptavidin coated beads (e.g., which may be used in simple centrifugal pulldown or spin filtration assays). Additionally or alternatively, the kits of the present technology may comprise recombinant, non-immune cells (e.g., HEK 293T) comprising any of the fusion proteins disclosed herein, and/or instructions for transducing the non-endogenous expression vectors (e.g., viral vectors) into non-immune and assaying for biotin-streptavidin binding competence via flow cytometry, cell sorting/purification, or immunohistochemistry.

The kits of the present technology can further comprise components necessary for detecting expression levels and/or activity of the reporter gene and/or the DOTA binding fragment of any embodiment of the fusion proteins disclosed herein. The kits can also contain a control sample or a series of control samples, which can be assayed and compared to the test sample. Each component of the kit can be enclosed within an individual sterile container (e.g., USP grade sterile glass vial sealed with a vinyl septum) and all of the various containers can be within a single package, along with instructions for interpreting the results of the assays performed using the kit. The kits of the present technology may contain a written product on or in the kit container. The written product describes how to use the reagents contained in the kit, e.g., for tracking or monitoring in vivo biodistribution, viability, and/or expansion of the recombinant immune cells. In certain embodiments, the use of the reagents can be according to the methods of the present technology.

In any embodiment disclosed herein, the biotinylated DOTA-based hapten may be of Formula I

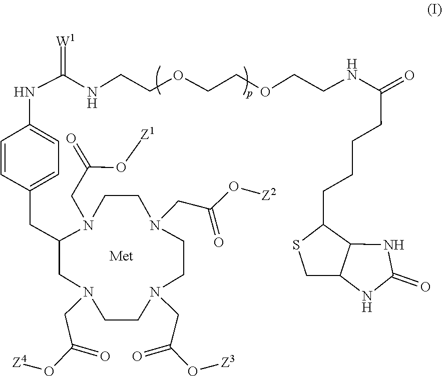

or a pharmaceutically acceptable salt thereof, wherein Met is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or a ¹⁶⁰Gd³⁺; W¹ is S or O; Z¹, Z², Z³, and Z⁴ are each independently a lone pair of electrons (i.e., providing an oxygen anion) or H; and p is 1, 2, 3, 4, 5, 6, 7, 8, 9, 10, 11, 12, 13, 14, 15, 16, 17, 18, 19, 20, 21, or 22. In any embodiment disclosed herein, the biotinylated DOTA-based hapten of Formula I may be of Formula IA

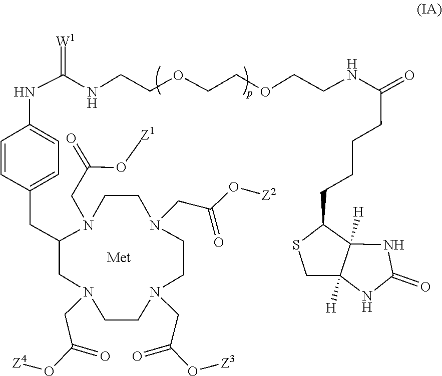

or a pharmaceutically acceptable salt thereof.

In any embodiment disclosed herein, the DOTA-bearing bischelate may be of Formula II

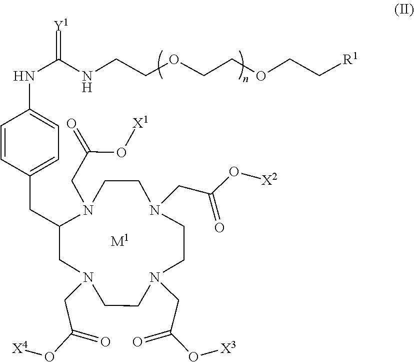

or a pharmaceutically acceptable salt thereof, wherein M1 is a chelated ¹⁷⁵Lu³⁺, ⁴⁵Sc³⁺, ⁶⁹Ga³⁺, ⁷¹Ga³⁺, ⁹⁸Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺,

### R¹ is

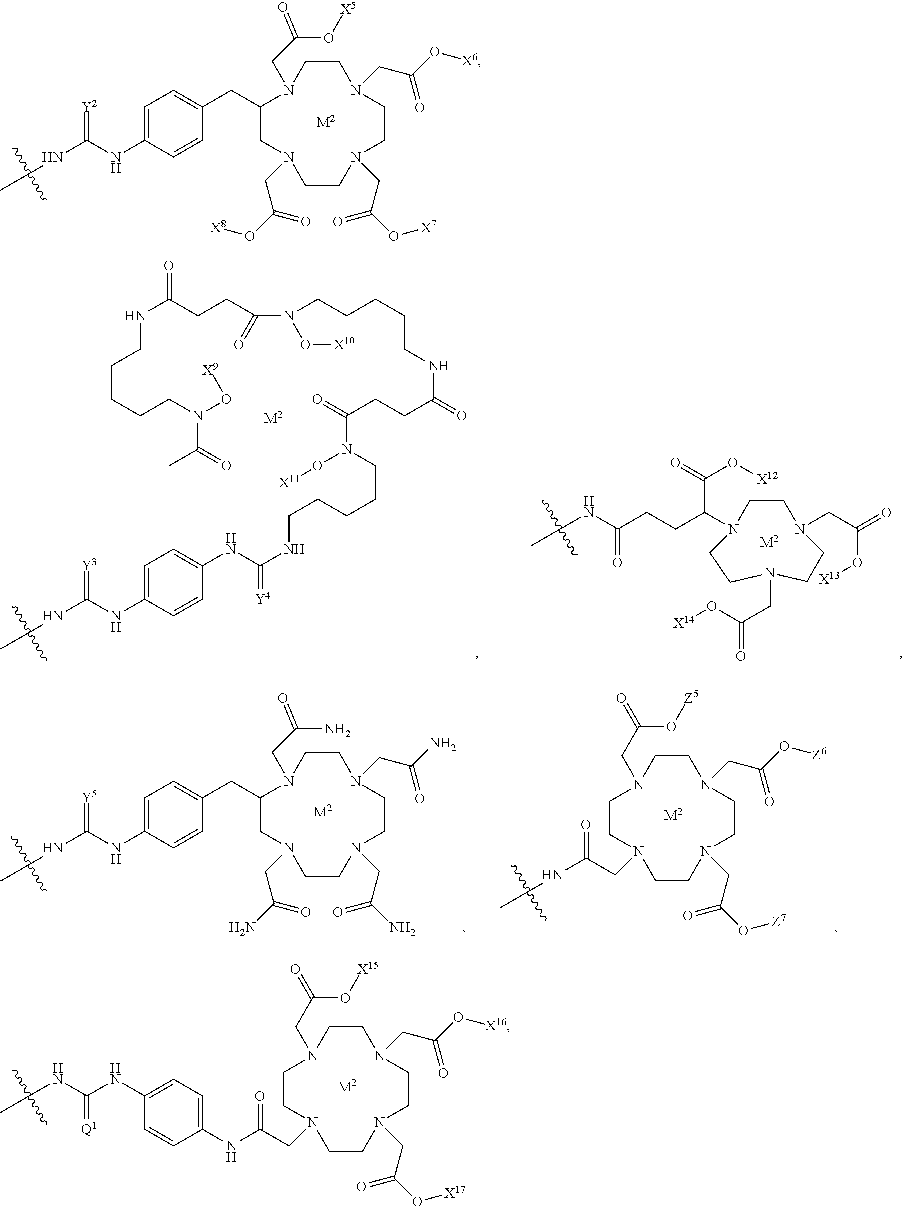

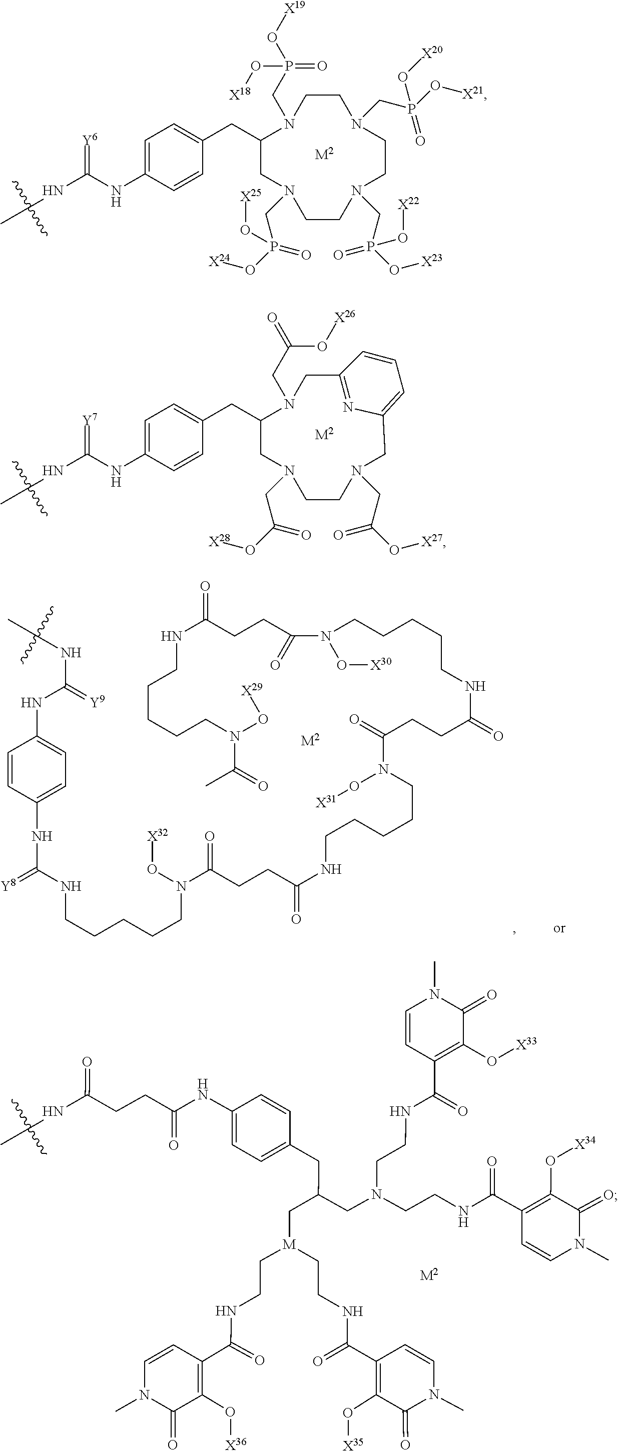

M² is independently at each occurrence a radionuclide cation chelated by the R¹ group; X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², X²³, X²⁴, X²⁵, X²⁶, X²⁷, X²⁸, X²⁹, X³⁰, X³¹, X³², X³³, X³⁴, X³⁵, and X³⁶ are each independently a lone pair of electrons (i.e., providing an oxygen anion) or H; Z⁵, Z⁶, and Z⁷ are each independently a lone pair of electrons (i.e., providing an oxygen anion) or H; Y², Y³, Y⁴, Y⁵, Y⁶, Y⁷, Y⁸ and Y⁹ are each independently S or O; Q¹ is S or O; and n is 1, 2, 3, 4, 5, 6, 7, 8, 9, 10, 11, 12, 13, 14, 15, 16, 17, 18, 19, 20, 21, or 22. In certain embodiments, n is 3. Additionally or alternatively, in some embodiments, the radionuclide cation is a divalent cation or a trivalent cation.

The DOTA-bearing bischelate of Formula II includes a radionuclide cation that is chelated by the R¹ group. The radionuclide cation may be an alpha particle-emitting isotope, a beta particle-emitting isotope, an Auger-emitter, or a combination of any two or more thereof. Examples of alpha particle-emitting isotopes include, but are not limited to, ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At and ²⁵⁵Fm. Examples of beta particle-emitting isotopes include, but are not limited to, ⁸⁶Y, ⁹⁰Y, ⁸⁹Sr, ¹⁶⁵Dy, ¹⁸⁶Re, ¹⁸⁸Re, ¹⁷⁷Lu, and ⁶⁷Cu. Examples of Auger-emitters include ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, and ²⁰³Pb. In any embodiment herein, the radionuclide cation may be ⁸⁹Zr, ⁶⁸Ga, ²⁰³Pb, ²¹²Pb, ²²⁷Th, or ⁶⁴Cu. In any embodiment herein, the radionuclide cation may be a divalent cation or a trivalent cation.

In any embodiment herein, the radionuclide cation may have a decay energy in the range of 20 to 6,000 keV. Decay energies can be within the range of 60 to 200 keV for an Auger emitter, 100-2,500 keV for a beta emitter, and 4,000-6,000 keV for an alpha emitter. Maximum decay energies of useful beta-particle-emitting radionuclides can range from 20-5,000 keV, 100-4,000 keV, or 500-2,500 keV. Decay energies of useful Auger-emitters can be <1,000 keV, <100 keV, or <70 keV. Decay energies of useful alpha-particle-emitting radionuclides can range from 2,000-10,000 keV, 3,000-8,000 keV, or 4,000-7,000 keV.

In one aspect, the present disclosure provides a method for tracking recombinant immune cells in a subject in vivo comprising: (a) administering to the subject an effective amount of any recombinant immune cell described herein, wherein the recombinant immune cell is configured to localize to a tissue expressing the target antigen recognized by the recombinant immune cell; (b) administering to the subject an effective amount of a DOTA-bearing bischelate, wherein the DOTA-bearing bischelate is configured to bind to the fusion protein expressed by the recombinant immune cell and comprises a radionuclide; and (c) determining the biodistribution of the recombinant immune cells in the subject by detecting radioactive levels emitted by the DOTA-bearing bischelate that are higher than a reference value. In another aspect, the present disclosure provides a method for tracking recombinant immune cells in a subject in vivo comprising: (a) administering to the subject an effective amount of a complex comprising any recombinant immune cell described herein and a DOTA-bearing bischelate comprising a radionuclide, wherein the complex is configured to localize to a tissue expressing the target antigen recognized by the recombinant immune cell; and (b) determining the biodistribution of recombinant immune cells in the subject by detecting radioactive levels emitted by the DOTA-bearing bischelate that are higher than a reference value.

In yet another aspect, the present disclosure provides a method for monitoring viability of recombinant immune cells in a subject comprising: (a) administering to the subject an effective amount of any recombinant immune cell described herein, wherein the recombinant immune cell is configured to localize to a tissue expressing the target antigen recognized by the recombinant immune cell; (b) administering to the subject an effective amount of a DOTA-bearing bischelate, wherein the DOTA-bearing bischelate is configured to bind to the fusion protein expressed by the recombinant immune cell and comprises a radionuclide; (c) detecting radioactive levels emitted by the DOTA-bearing bischelate that are higher than a reference value at a first time point; (d) detecting radioactive levels emitted by the DOTA-bearing bischelate that are higher than a reference value at a second time point; and (e) determining that the recombinant immune cells in the subject are viable when the radioactive levels emitted by the DOTA-bearing bischelate at the second time point are comparable to that observed at the first time point. In some embodiments, the method further comprises administering to the subject a second effective amount of the DOTA-bearing bischelate prior to step (d). Also disclosed herein is a method for monitoring viability of recombinant immune cells in a subject comprising: (a) administering to the subject an effective amount of a complex comprising any recombinant immune cell described herein and a DOTA-bearing bischelate comprising a radionuclide, wherein the complex is configured to localize to a tissue expressing the target antigen recognized by the recombinant immune cell; (b) detecting radioactive levels emitted by the DOTA-bearing bischelate that are higher than a reference value at a first time point; (c) detecting radioactive levels emitted by the DOTA-bearing bischelate that are higher than a reference value at a second time point; and (d) determining that the recombinant immune cells in the subject are viable when the radioactive levels emitted by the DOTA-bearing bischelate at the second time point are comparable to that observed at the first time point.

In yet another aspect, the present disclosure provides a method for monitoring expansion of recombinant immune cells in a subject comprising: (a) administering to the subject an effective amount of any recombinant immune cell described herein, wherein the recombinant immune cell is configured to localize to a tissue expressing the target antigen recognized by the recombinant immune cell; (b) administering to the subject a first effective amount of a DOTA-bearing bischelate, wherein the DOTA-bearing bischelate is configured to bind to the fusion protein expressed by the recombinant immune cell and comprises a radionuclide; (c) detecting radioactive levels emitted by the DOTA-bearing bischelate that are higher than a reference value at a first time point; (d) administering to the subject a second effective amount of the DOTA-bearing bischelate after step (c); (e) detecting radioactive levels emitted by the DOTA-bearing bischelate that are higher than a reference value at a second time point; and (f) determining that the recombinant immune cells in the subject have expanded when the radioactive levels emitted by the DOTA-bearing bischelate at the second time point are higher relative to that observed at the first time point.

In any and all embodiments of the methods disclosed herein, the radioactive levels emitted by the complex or the DOTA-bearing bischelate are detected using positron emission tomography (PET) or single photon emission computed tomography (SPECT).

The DOTA-bearing bischelate may be administered at any time between 1 minute to 4 or more days following administration of the recombinant immune cells expressing any of the fusion proteins disclosed herein. For example, in some embodiments, the DOTA-bearing bischelate is administered 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, or any range therein, following administration of the recombinant immune cells expressing any of the fusion proteins disclosed herein. Alternatively, the DOTA-bearing bischelate may be administered at any time after 4 or more days following administration of the recombinant immune cells expressing any of the fusion proteins disclosed herein.

Additionally or alternatively, in some embodiments of the methods disclosed herein, the DOTA-bearing bischelate is administered intravenously, intramuscularly, intraarterially, intrathecally, intracranially, intracapsularly, intraorbitally, intradermally, intraperitoneally, transtracheally, subcutaneously, intracerebroventricularly, orally, intratumorally, or intranasally. In certain embodiments, the DOTA-bearing bischelate is administered into the cerebral spinal fluid or blood of the subject.

In some embodiments of the methods disclosed herein, the radioactive levels emitted by the DOTA-bearing bischelate are detected between 2 to 120 hours after the DOTA-bearing bischelate is administered. In certain embodiments of the methods disclosed herein, the radioactive levels emitted by the DOTA-bearing bischelate are expressed as the percentage injected dose per gram tissue (% ID/g). The reference value may be calculated by measuring the radioactive levels present in normal tissues, and computing the average radioactive levels present in normal tissues±standard deviation. In some embodiments, the reference value is the standard uptake value (SUV). See Thie J A, J Nucl Med. 45(9):1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

In any and all embodiments of the methods disclosed herein, the radionuclide is an alpha particle-emitting isotope, a beta particle-emitting isotope, or an Auger-emitter. Examples of radionuclides include ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu. In any of the preceding embodiments of the methods disclosed herein, the subject is human.

In any and all embodiments of the methods disclosed herein, the subject is diagnosed with, or is suspected of having cancer. Examples of cancer include, but are not limited to, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

### BRIEF DESCRIPTION OF THE DRAWINGS

FIGS. 1A-1C show the workflow for the kits of the present technology for CAR-T cells. The kits disclosed herein include three components: (1) an expression vector system (e.g., a viral vector) that can be used to transduce immune cells (e.g., CAR T cells) that express a membrane-bound DOTA binding scFv fragment that captures DOTA-based haptens (e.g., radioactive or non-radioactive) (see FIG. 1A); (2) a biotinylated DOTA-based hapten for Fluorescence-activated cell sorting (FACS) characterization to test the number of DOTA binding sites transduced per cell, as well as histological assays (see FIGS. 1A-1B); and (3) a DOTA-bearing bischelate for visualizing and assessing the quantitative biodistribution of the transduced immune cells in vivo (see FIG. 1C, illustrating via use of PET imaging).

FIGS. 2A-2B shows exemplary DOTA-bearing bischelates and biotinylated DOTA-based haptens useful in the kits of the present technology. The biotinylated DOTA-based hapten contained in vial 2 is useful for in vitro flow cytometry characterization of transduced CAR-T cells to verify successful transduction with the expression vector system of vial 1, and to assay for functional binding to DOTA-based haptens (example of a biotinylated DOTA-based hapten illustrated in FIG. 2A). The DOTA-bearing bischelate present in vial 3 may be utilized for in vivo imaging using quantitative non-invasive PET following injection of the transduced CAR T cells into a patient. Exemplary DOTA-bearing bischelates are provided in FIG. 2B illustrating the long-lived PET emitter Zr-89 (half-life: 3.3 days) for multi-day tracking (top) and/or the short-lived PET emitter Ga-68 (half-life: 68 minutes) for single-snapshot tracking (bottom). Zr-89 and Ga-68 are used in nuclear medicine for radiolabeling of antibodies and small molecules, respectively.

FIGS. 3A-3C show three different strategies to virally transduce primary human T cells with both C825 and CD19-CAR. FIG. 3A show transduction with two single constructs, one encoding C825 with a GFP reporter (top) and one encoding the CD19 CAR (bottom). FIG. 3B shows a bicistronic construct encoding C825 with a transmembrane domain and GFP reporter and CD19 CAR, separated by P2A cleavage site. FIG. 3C shows a bicistronic construct encoding C825 with a Thy1 GPI linkage and His tag reporter and CD19 CAR, separated by a P2A cleavage site. Representative flow plots of transduction of primary human T cells are shown on the right.

FIG. 4 shows representative theranostic radiohaptens for SPECT (In-111), PET (Zr-89), and alpha-therapy (Ac-225 and Pb-212). Alternatively, DOTA-hapten [¹⁷⁷Lu]Lu-DOTA-benzene (see FIG. 1) can be used for theranostic beta-therapy.

FIG. 5A shows a maximum intensity projection (MIP) PET/CT of a mouse bearing bilateral 293T only/293T-C825 xenografts in the upper shoulders approximately 18 h post-injection of [⁸⁶Y]DOTA-benzene. 293T cells exhibit a cancer stem cell-like phenotype when cultured as 3D spheres, as well as form phenotypically stable xenografts with histological appearance of high-grade, poorly differentiated tumor. The 293T-C825 tumor uptake determined by ex vivo biodistribution immediately following imaging was ˜14% ID/g. FIG. 5B shows an in vitro saturation binding assay for 293T-C825 cells (solid line) and 293T cells (dotted line) for radiohapten capture. FIG. 5C shows a comparison of biodistribution of tracer pretargeted [²²⁵Ac]Pr-DOTA or [¹¹¹In]Pr-DOTA in groups of SW1222 human colorectal cancer tumor-bearing athymic nude mice at 24 h p.i. Following i.v. injections of huA33-C825 bispecific antibody (0.25 mg, 1.19 nmol), clearing agent, and radiolabeled DOTA haptens, the animals were euthanized 24 h later for organ collection and assayed for radioactivity. Data is presented as mean±SEM. FIG. 5D shows an MIP SPECT/CT image approximately 24 h p.i. of pretargeted [¹¹¹In]Pr-DOTA in a SW1222 tumor-bearing athymic nude mouse. The SW1222-xenograft can be clearly delineated in the flank.

FIGS. 6A-6B show radiohapten capture imaging results of C825-CD19 CAR-T cells with membrane expression of C825 (a picomolar binding affinity, hapten capture scFv antibody). FIG. 6A shows in vitro saturation binding curve using [In]Pr-DOTA binding to quantify C825 expression in C825-CD19 CAR-T cells. FIG. 6B shows a NSG mouse with a s.c. Raji GFP-fluc tumor in the right shoulder. Ten days after i.v. CAR-T cell injection (2.5×10⁶), the mouse was injected with ¹¹¹In-radiohapten for in vivo tracking of CAR T cells (either: CD19 CAR+C825, or control CD19 CAR only). FIG. 6B shows SPECT/CT images collected 18 h after injection of ¹¹¹In-radiohapten. Shown are MIP images for animals treated with C825+CD19 CAR T cells, or control CD19 CAR T cells only.

FIG. 7 shows an exemplary FACS assay for functional C825-hapten binding using GPA33×C825 bispecific antibodies.

FIG. 8 shows BIACORE assay results at 10 nM concentration.

FIG. 9A shows schematic structures of retroviral vectors SFG-Thor, SFG-19BBz (CAR) and SFG-C825. FIG. 9B shows that there is no difference between SFG-Thor T cells and SFG-19BBz (CAR) T cells with respect to killing CD19(+) Raji tumor cells as measured by in vitro 4 h cytotoxicity assays. SFG-C825 and non-transduced (NT) T cells are included as negative controls. (n=3-4 donors). FIG. 9C shows in vitro binding of [¹¹¹In]InPr at 1 h. This representative data set demonstrates the specific binding of the radiolabeled DOTA probe to C825-expressing T cells, whereas no significant uptake was observed in SFG-19BBz (CAR) and NT T cells. (All experiments were performed in triplicate at 37° C.). Data are mean±SD. FIG. 9D shows in vitro binding kinetics of [¹¹¹In]InPr to SFG-Thor T cells (n=3 independent assays; representative example shown). FIG. 9E shows an exemplary scheme of in vivo study for assessing T cell targeting to tumor cells. ⁶⁸Ga-NODAGA-Pr (100 mCu, 700 pmol) was used as the radiotracer and administered 10 days after T cell administration (1×10⁶). FIG. 9F shows exemplary Maximum intensity projection (MIP) images at 1 h post-injection (p.i.) of ⁶⁸Ga-NODAGA-Pr depicting homing and accumulation of SFG-Thor T cells at the tumor (right shoulder, red arrow). No uptake above background at the tumor site is noted following SFG-19BBz (CAR) T cell administration (blue arrow). FIG. 9G shows mean uptake in tumors and tumor-to-normal-tissue-ratios (TNR) (SFG-Thor: n=4; SFG-19BBz (CAR): n=2) using image-based biodistribution. **, P<0.01.

FIGS. 10A-10B show in vivo tracking of the engineered CAR T cells of the present technology with ⁸⁶Y-DOTABn. FIG. 10A shows an exemplary scheme for tracking engineered T cells in vivo in a s.c. Raji-tumor mouse model (3×10⁶ cells) with established treatment failure. Seven days post tumor inoculation, mice were injected i.v. with either 3×10⁶ huC825-19BBz or 3×10⁶ 19BBz T cells. On day 17 post T cell administration, mice demonstrating persistent growing tumor burden indicating treatment failure were i.v. injected with ⁸⁶Y-DOTA-Bn (3.7 MBq; 40 pmol) to assess persistence and localization of the transplanted T cells. FIG. 10B shows Maximum intensity projection (MIP) and axial PET/CT images at 1, 3 and 16 h p.i. depict accumulation of huC825-19BBz-CAR T cells at the tumor (orange circle). Highest intratumoral T cell uptake was seen at 3 h pi of 4.9% ID/g (vs 0.8% ID/g in control). No uptake above background at the tumor is noted in control mice (19BBz CAR; green circle). Rapid, predominant renal tracer clearance was noted.

FIG. 11 shows representative flow cytometry assay of capture by cell surface-anchored huC825.

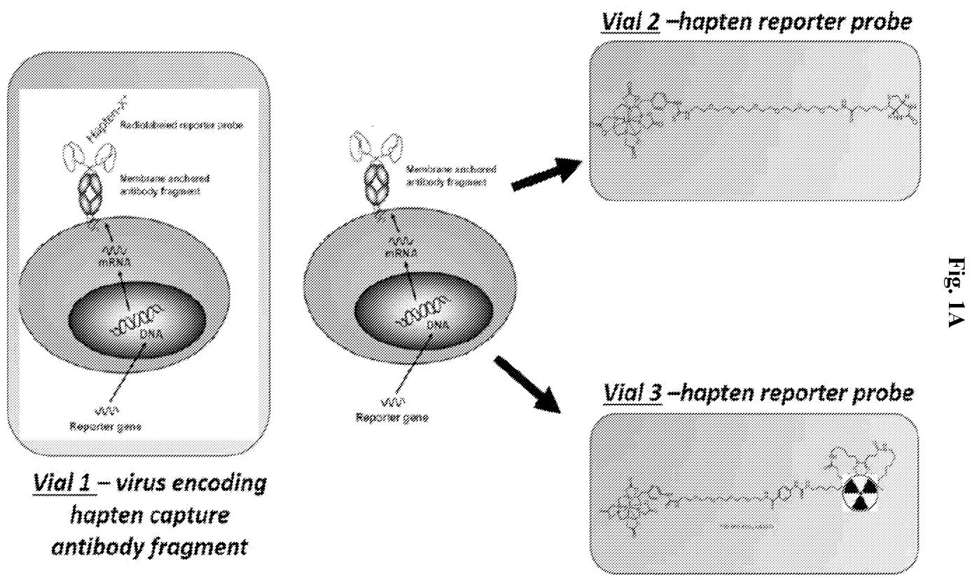

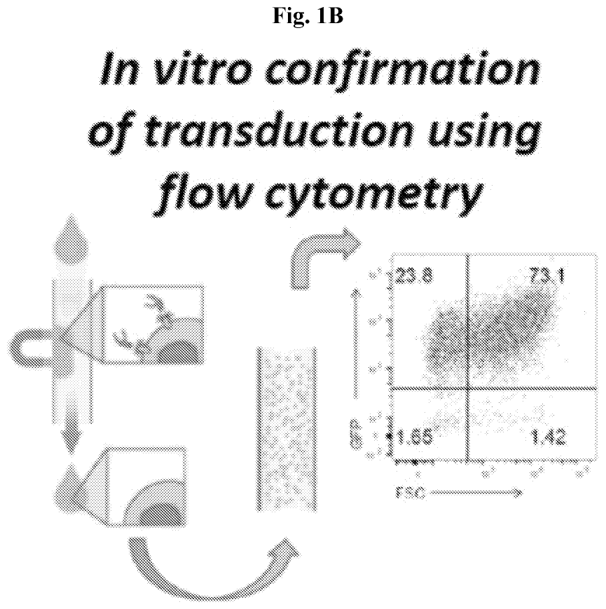

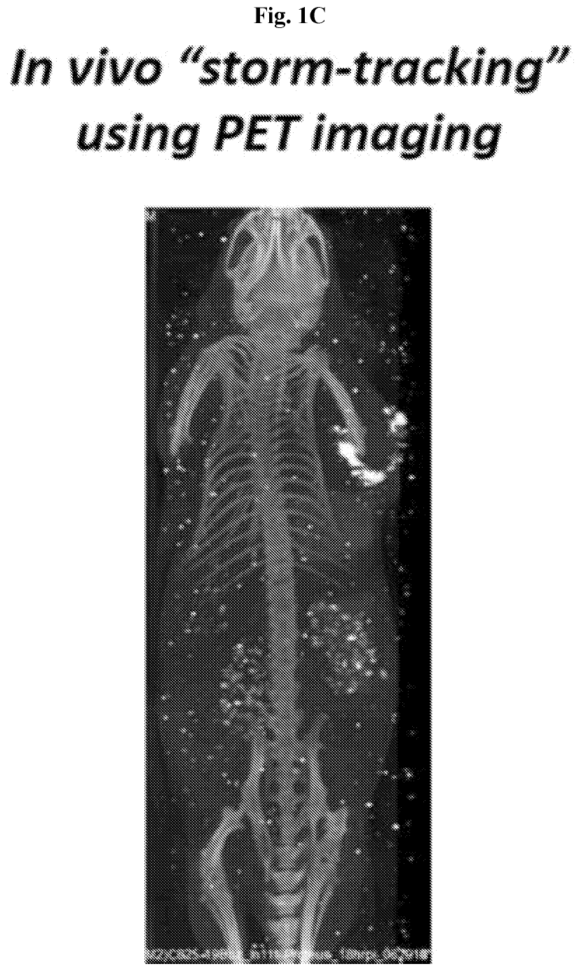

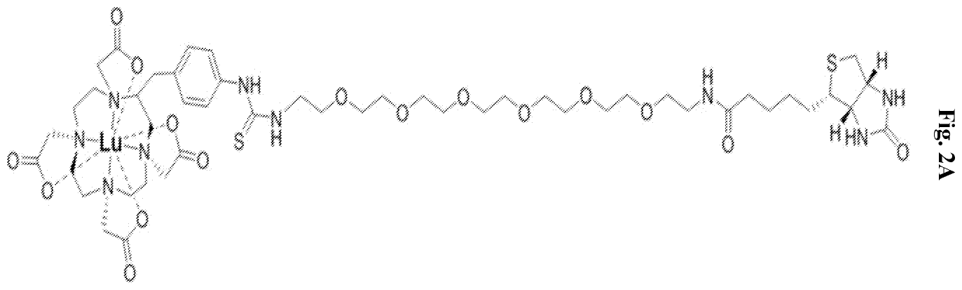

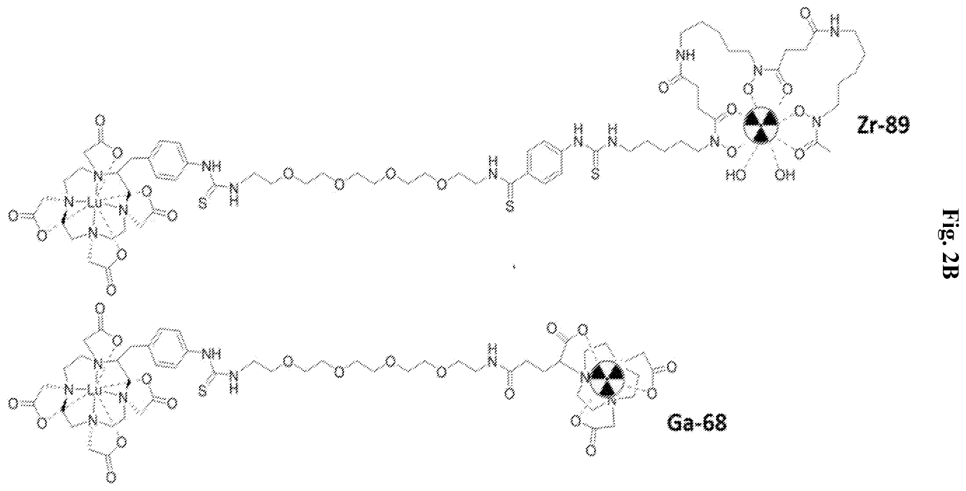

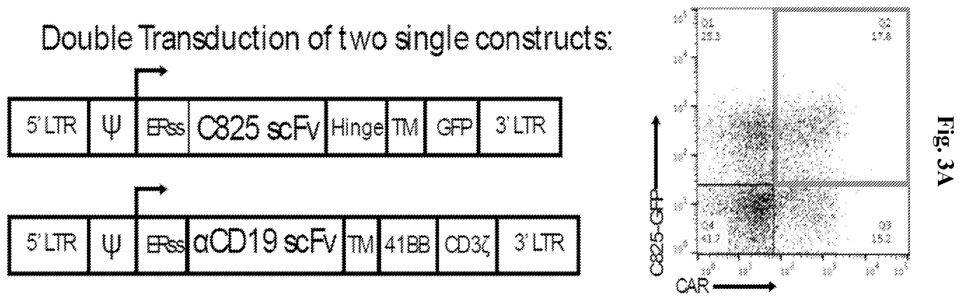

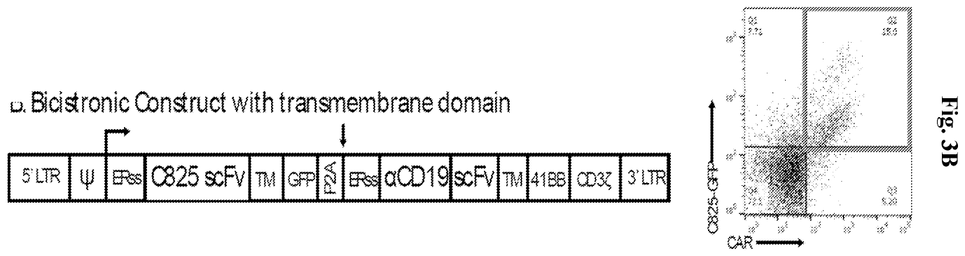

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure provides kits for rapid transduction of immune cells (e.g., CAR T cells) to create specialized reporter cells that are useful for monitoring the in vivo biodistribution, viability, and expansion of immune cells having any target specificity employed as an immunologic cancer therapy. The tracker system includes three components: (1) an expression vector system (e.g., a viral vector) that can be used to transduce immune cells (e.g., CAR T cells) that express a membrane-bound DOTA binding scFv fragment that captures DOTA-based haptens (e.g., radioactive or non-radioactive); (2) a biotinylated DOTA-based hapten for Fluorescence-activated cell sorting (FACS) characterization to test the number of DOTA binding sites transduced per cell, as well as histological assays; and (3) a DOTA-bearing bischelate for visualizing and assessing the quantitative biodistribution of the transduced immune cells in vivo.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The phrase “and/or” as used in the present disclosure will be understood to mean any one of the recited members individually or a combination of any two or more thereof—for example, “A, B, and/or C” would mean “A, B, C, A and B, A and C, or B and C.”

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), intracranially, rectally, intrathecally, intratumorally or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a DOTA hapten will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; nanobodies; camelids; linear antibodies; single-chain antibody molecules; and multi-specific antibodies formed from antibody fragments.

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFᵥ)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody can selectively bind. The antigen may be a protein, carbohydrate, nucleic acid, lipid, hapten, or other naturally occurring or synthetic compound. In some embodiments, the target antigen may be a DOTA-based hapten or a tumor antigen. An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, “Bₘₐₓ” is the total density (concentration) of receptors in a sample of tissue.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. Examples of cancers include, but are not limited to, neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

“Detectable label” as used herein refers to a molecule or a compound or a group of molecules or a group of compounds used to identify a nucleic acid, protein, molecule, or compound of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired effect (e.g., a diagnostic or therapeutic/prophylactic effect). In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, the epitope is a conformational epitope or a non-conformational epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

The term “Fe region”, “Fc domain”, or “Fc fragment” as used herein refers to a C-terminal region of an immunoglobulin heavy chain, which is capable of binding to a mammalian Fc(gamma) or Fc(Rn) receptor, human Fc(gamma) or Fc(Rn) receptor. An Fc receptor (Felt) refers to a receptor that binds to an Fc fragment or the Fc region of an antibody. In certain embodiments, the FcR is a native human FcR sequence. In some embodiments, the FcR binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIII (an “inhibiting receptor”), which have similar amino acid sequences that differ primarily in the cytoplasmic domains thereof. FcRs are described in Ravetch and Kinet, 1991, Ann. Rev. Immunol., 9:457-92; Capel et al., 1994, Immunomethods, 4:25-34; and de Haas el al., 1995, J. Lab. Clin. Med., 126:330-41. “FcR” also includes the neonatal receptor, FcRn, which is responsible for the transfer of maternal IgGs to the fetus (Guyer et al., 1976 J. Immunol., 117:587 and Kim et al 1994, J. Immunol., 24:249) and contributes to the prolonged in vivo elimination half-lives of antibodies and Fc-fusion proteins in vivo. The Fc fragment, region, or domain may be a native sequence Fc region. Although the boundaries of the Fe region of an immunoglobulin heavy chain might vary, the human IgG heavy chain Fe region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a DOTA binding fragment (e.g., C825 scFv), a transmembrane domain or a glycosylphosphatidylinositol (GPI)-anchored polypeptide (e.g., Thy1), and optionally a reporter gene. In some embodiments, the fusion protein optionally comprises a spacer domain (e.g., an IgG Fc domain). Fusion of a DOTA binding fragment (e.g., C825 scFv) with a transmembrane domain or glycosylphosphatidylinositol (GPI)-anchored polypeptide, and optionally a reporter gene results in a membrane-bound DOTA binding molecule that permits the specific capture of detectably labelled DOTA-based haptens. Accordingly, the fusion proteins described herein are useful in characterizing in vitro and in vivo properties of radioactive or non-radioactive DOTA-based hapten probes, such as uptake, pharmacokinetics (e.g., affinity), biodistribution, specificity, cytotoxicity, and the like. In some embodiments, a fusion protein is in a complex with, or is in association with a radioactive or non-radioactive DOTA-based hapten probe. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4.sup.th ed., Cold Spring Harbor Laboratory Press, Cold Spring Harbor, N.Y. (2012)), the entire contents of which are incorporated herein by reference.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, a “heterologous nucleic acid sequence” is any nucleic acid sequence placed at a location where it does not normally occur. A heterologous nucleic acid sequence may comprise a sequence that does not naturally occur in a cell, or it may comprise only sequences naturally found in the cell, but placed at a non-normally occurring location in the cell. In some embodiments, the heterologous nucleic acid sequence is not an endogenous sequence. In certain embodiments, the heterologous nucleic acid sequence is an endogenous sequence that is derived from a different cell. In other embodiments, the heterologous nucleic acid sequence is a sequence that occurs naturally in a cell but is then relocated to another site where it does not naturally occur, rendering it a heterologous sequence at that new site.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Leski. J. Mol. Biol. 196:901-917 (1987)).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, MD. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Leski. J. Mol. Biol. 196:901-917 (1987)).

As used herein, the terms “individual”, “patient”, or “subject” can be an individual organism, a vertebrate, a mammal, or a human. In some embodiments, the individual, patient or subject is a human.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, “linker” typically refers to a portion of a molecule or entity that connects two or more different regions of interest (e.g., particular structural and/or functional domains or moieties of interest). The linker may lack a defined or rigid structure and/or may not materially alter the relevant function of the domain(s) or moiety(ies) within the two or more different regions of interest. In some embodiments, the linker is or comprises a polypeptide and may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids long. In certain embodiments, a polypeptide linker may have an amino acid sequence that is or comprises GGGGSGGGGSGGGGS (i.e., [G₄S]₃) (SEQ ID NO: 9), GGGGSGGGGSGGGGSGGGGS (i.e., [G₄S]₄) (SEQ ID NO: 10), or GGGGSGGGGSGGGGSGGGGSGGGGSGGGGS (i.e., [G₄S]₆) (SEQ ID NO: 11).

As used herein, “operably linked” means that expression control sequences are positioned relative to a nucleic acid of interest to initiate, regulate or otherwise control transcription of the nucleic acid of interest. In some embodiments, transcription of a polynucleotide operably linked to an expression control element (e.g., a promoter) is controlled, regulated, or influenced by the expression control element.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the cell of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the cell of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a cell. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, a “reporter gene” refers to a polynucleotide sequence encoding a gene product (e.g., polypeptide) that can generate, under appropriate conditions, a detectable signal that allows detection of the presence and/or quantity of the gene product.

As used herein, a “spacer domain” is a polypeptide that links two distinct regions or domains of a protein (e.g., the distinct regions of a fusion protein). In some embodiments, spacer domains have no specific biological activity, and their purpose is simply to link two protein domains, or to preserve the minimum distance or spatial relationship between said protein domains. Additionally or alternatively, is some embodiments, the constituent amino acids of the spacer domains may be selected based on physicochemical properties, such as flexibility, hydrophilicity, net charge, proteolytic sensitivity or lack thereof, and lack of immunogenicity.

As used herein, “specifically binds” refers to a molecule (e.g., an antibody or antigen binding fragment thereof) which recognizes and binds another molecule (e.g., an antigen or hapten), but that does not substantially recognize and bind other molecules. The terms “specific binding,” “specifically binds to,” or is “specific for” a particular molecule (e.g., an antigen, or an epitope on an antigen, or hapten), as used herein, can be exhibited, for example, by a molecule having a K_{D} for the molecule to which it binds to of about 10⁻⁴ M, 10⁻⁵M, 10⁻⁶M, 10⁻⁷ M, 10⁻⁸M, 10⁻⁹M, 10⁻¹⁰ M, 10⁻¹¹ M or 10⁻¹²M. The term “specifically binds” may also refer to binding where a molecule (e.g., an antibody or antigen binding fragment thereof) binds to a particular antigen, or an epitope on a particular antigen, or hapten, without substantially binding to any other antigen, epitope, or hapten.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, a “vector” refers to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

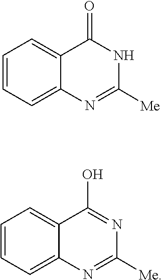 [SMILES: Cc1nc2ccccc2c(=O)[nH]1 ; Cc1nc(O)c2ccccc2n1]

As another example, guanidines may exhibit the following isomeric forms in protic organic solution (e.g., water), also referred to as tautomers of each other:

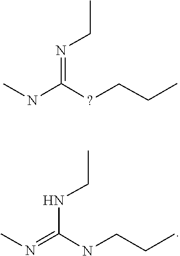

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

### Kits

The present technology provides kits for use in any of the methods described herein. In one aspect, the present disclosure provides kits including any of the recombinant immune cells disclosed herein and instructions for tracking or monitoring in vivo biodistribution, viability, and/or expansion of the recombinant immune cells.

In one aspect, the kits may include non-endogenous expression vectors comprising recombinant nucleic acids encoding any of the fusion proteins disclosed herein, and instructions for transducing the non-endogenous expression vectors (e.g., viral vectors) into immune cells and tracking or monitoring in vivo biodistribution, viability, and/or expansion of the transduced recombinant immune cells. The immune cells may be derived from an autologous donor or an allogenic donor. Additionally or alternatively, the kits of the present technology further comprise a biotinylated DOTA-based hapten; and one or more DOTA-bearing bischelate(s). The biotinylated DOTA-based hapten is useful for FACS, quantification of DOTA binding sites in transfected immune cells, and histology assays. In some embodiments, the one or more DOTA-bearing bischelate(s) in the kit may be labelled with a suitable radionuclide.

Additionally or alternatively, in some embodiments, at least one of the non-endogenous expression vectors, the biotinylated DOTA-based hapten; and one or more DOTA-bearing bischelate(s) may be stored in lyophilized form. In any of the embodiments disclosed herein, the kits can also comprise, e.g., one or more of buffering salts (e.g., for radiochemistry and/or reconstituting the lyophilized contents of the kit to generate isotonic solutions suitable for use in cell assays or ex vivo inoculation), excipients, radioprotectants, preservatives, stabilizing agents, cell culture medium, cell culture supplements, ITLC strips, and the like. Excipients that may aid in resuspension, stability and shelf life of the non-endogenous expression vectors (e.g., viral vectors), biotinylated DOTA-based hapten or DOTA-bearing bischelate(s) include ascorbic acid (as an antioxidant), sodium benzoate (e.g., 0.5% sodium benzoate as a preservative) and optionally stabilizers to preserve viral infectivity and titer such as sorbitol (e.g., 10% sorbitol). Examples of buffers that are compatible with radiochemistry are NH₄OAc (ammonium acetate), NaOAc, (sodium acetate), or HEPES (4-(2-hydroxyethyl)-1-piperazineethanesulfonic acid). The typical patient dose for radiolabeled DOTA-bearing bischelate(s) ranges from 5-10 mCi for imaging applications. The one or more DOTA-bearing bischelate(s) contained in the kit may be present in an amount sufficient for one or patient doses, as well as quality control testing by radio-instant TLC or radio-HPLC.

The kits of the present technology can further comprise components necessary for determining biotin-streptavidin binding competence of the biotinylated DOTA-based hapten, such as streptavidin coated beads (e.g., which may be used in simple centrifugal pulldown or spin filtration assays). Additionally or alternatively, the kits of the present technology may comprise recombinant, non-immune cells (e.g., HEK 293T) comprising any of the fusion proteins disclosed herein, and/or instructions for transducing the non-endogenous expression vectors (e.g., viral vectors) into non-immune and assaying for biotin-streptavidin binding competence via flow cytometry, cell sorting/purification, or immunohistochemistry.

The kits of the present technology can further comprise components necessary for detecting expression levels and/or activity of the reporter gene and/or the DOTA binding fragment of any embodiment of the fusion proteins disclosed herein. The kits can also contain a control sample or a series of control samples, which can be assayed and compared to the test sample. Each component of the kit can be enclosed within an individual sterile container (e.g., USP grade sterile glass vial sealed with a vinyl septum) and all of the various containers can be within a single package, along with instructions for interpreting the results of the assays performed using the kit. The kits of the present technology may contain a written product on or in the kit container. The written product describes how to use the reagents contained in the kit, e.g., for tracking or monitoring in vivo biodistribution, viability, and/or expansion of the recombinant immune cells. In certain embodiments, the use of the reagents can be according to the methods of the present technology.

### Biotinylated DOTA-Based Haptens of the Present Technology

In any embodiment disclosed herein, the biotinylated DOTA-based hapten may be of Formula I

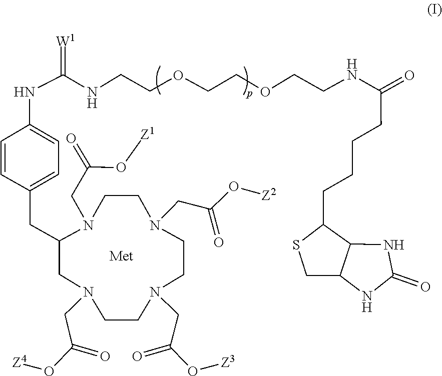

or a pharmaceutically acceptable salt thereof, wherein Met is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or a ¹⁶⁰Gd³⁺; W¹ is S or O; Z¹, Z², Z³, and Z⁴ are each independently a lone pair of electrons (i.e., providing an oxygen anion) or H; and p is 1, 2, 3, 4, 5, 6, 7, 8, 9, 10, 11, 12, 13, 14, 15, 16, 17, 18, 19, 20, 21, or 22. In any embodiment disclosed herein, the biotinylated DOTA-based hapten of Formula I may be of Formula IA

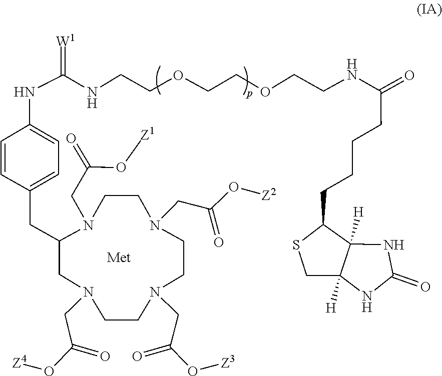

or a pharmaceutically acceptable salt thereof.

### DOTA-Bearing Bischelates of the Present Technology

In any embodiment disclosed herein, the DOTA-bearing bischelate may be of Formula II

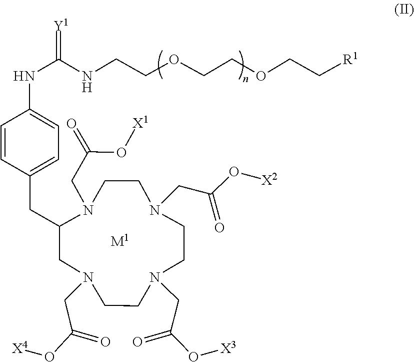

or a pharmaceutically acceptable salt thereof, wherein M¹ is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;

### R1 is

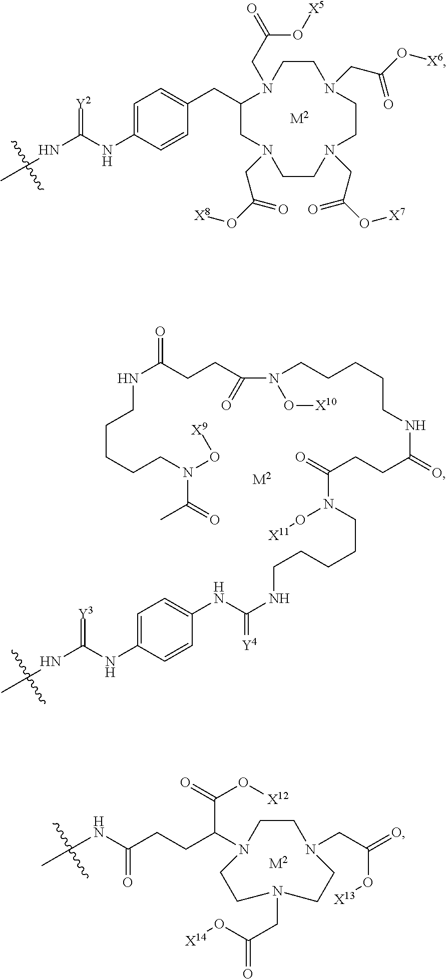

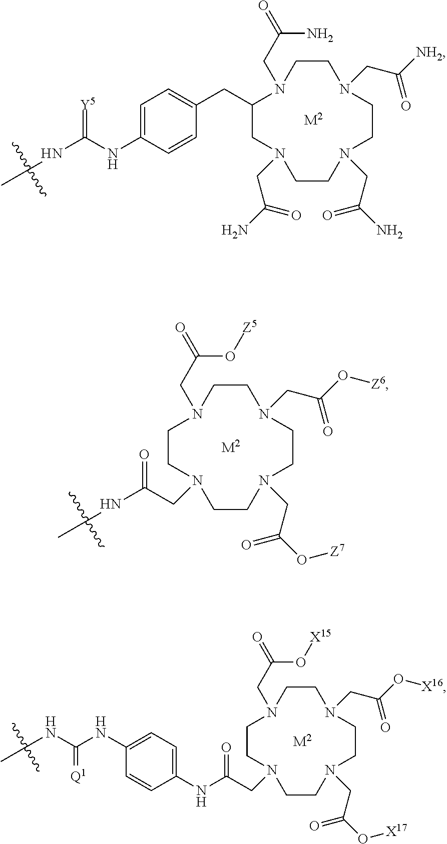

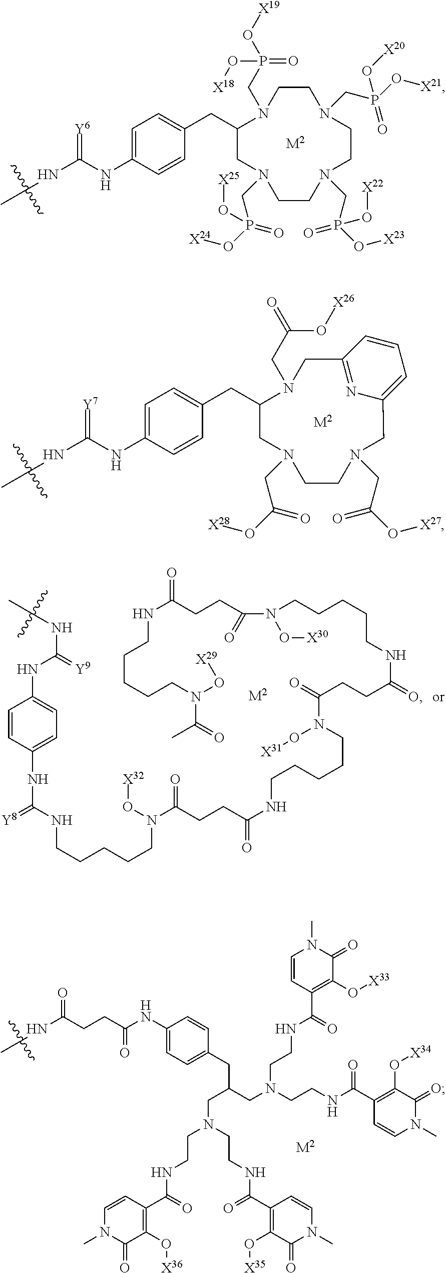

M² is independently at each occurrence a radionuclide cation chelated by the R¹ group; X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², X²³, X²⁴, X²⁵, X²⁶, X²⁷, X²⁸, X²⁹, X³⁰, X³¹, X³², X³³, X³⁴, X³⁵, and X³⁶ are each independently a lone pair of electrons (i.e., providing an oxygen anion) or H; Z⁵, Z⁶, and Z⁷ are each independently a lone pair of electrons (i.e., providing an oxygen anion) or H; Y¹, Y², Y³, Y⁴, Y⁵, Y⁶, Y⁷, Y⁸ and Y⁹ are each independently S or O; Q¹ is S or O; and n is 1, 2, 3, 4, 5, 6, 7, 8, 9, 10, 11, 12, 13, 14, 15, 16, 17, 18, 19, 20, 21, or 22. In certain embodiments, n is 3. Additionally or alternatively, in some embodiments, the radionuclide cation is a divalent cation or a trivalent cation.

The DOTA-bearing bischelate of Formula II includes a radionuclide cation that is chelated by the R¹ group. The radionuclide cation may be an alpha particle-emitting isotope, a beta particle-emitting isotope, an Auger-emitter, or a combination of any two or more thereof. Examples of alpha particle-emitting isotopes include, but are not limited to, ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, and ²⁵⁵Fm. Examples of beta particle-emitting isotopes include, but are not limited to, ⁸⁶Y, ⁹⁰Y, ⁸⁹Sr, ¹⁶⁵Dy, ¹⁸⁶Re, ¹⁸⁸Re, ¹⁷⁷Lu, and ⁶⁷Cu. Examples of Auger-emitters include ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, and ²⁰³Pb. In any embodiment herein, the radionuclide cation may be ⁸⁹Zr, ⁶⁸Ga, ²⁰³Pb, ²¹²Pb, ²²⁷Th, or ⁶⁴Cu. In any embodiment herein, the radionuclide cation may be a divalent cation or a trivalent cation.

In any embodiment herein, the radionuclide cation may have a decay energy in the range of 20 to 6,000 keV. Decay energies can be within the range of 60 to 200 keV for an Auger emitter, 100-2,500 keV for a beta emitter, and 4,000-6,000 keV for an alpha emitter. Maximum decay energies of useful beta-particle-emitting radionuclides can range from 20-5,000 keV, 100-4,000 keV, or 500-2,500 keV. Decay energies of useful Auger-emitters can be <1,000 keV, <100 keV, or <70 keV. Decay energies of useful alpha-particle-emitting radionuclides can range from 2,000-10,000 keV, 3,000-8,000 keV, or 4,000-7,000 keV.

### Compositions Including Fusion Proteins of the Present Technology

The fusion proteins of the present technology comprise a humanized DOTA binding fragment fused to a first transmembrane domain, or a glycosylphosphatidylinositol (GPI)-anchored polypeptide. The humanized DOTA binding fragment comprises a heavy chain immunoglobulin variable domain (V_{H}) sequence and a light chain immunoglobulin variable domain (V_{L}) sequence of SEQ ID NO: 1 and SEQ ID NO: 5, respectively. The V_{H} domain sequence may be located at the N-terminus or the C-terminus of the V_{L} domain sequence.

huC825 V_{H}
(SEQ ID NO: 1)
HVQLVESGGGLVQPGGSLRLSCAASGFSLTDYGVHWVRQAPGKGLEWLG
VIWSGGGTAYNTALISRFTISRDNSKNTLYLQMNSLRAEDTAVYYCARR
GSYPYNYFDAWGCGTLVTVSS
huC825 V_{L}
(SEQ ID NO: 5)
QAVVTQEPSLTVSPGGTVTLTCGSSTGAVTASNYANWVQQKPGQCPRGL
IGGHNNRPPGVPARFSGSLLGGKAALTLLGAQPEDEAEYYCALWYSDHW
VIGGGTKLTVLG

The V_{H} CDR1, V_{H} CDR2 and V_{H} CDR3 sequences of SEQ ID NO: 1 are DYGVH (SEQ ID NO: 2), VIWSGGGTAYNTALIS (SEQ ID NO: 3), RGSYPYNYFDA (SEQ ID NO: 4), respectively, and are underlined in order of appearance. The V_{L} CDR1, V_{L} CDR2 and V_{L} CDR3 sequences of SEQ ID NO: 5 are GSSTGAVTASNYAN (SEQ ID NO: 6), GHNNRPP (SEQ ID NO: 7), and ALWYSDHWV (SEQ ID NO: 8), respectively, and are underlined in order of appearance. Additionally or alternatively, in some embodiments, the sequence of an intra-peptide linker between the V_{H} domain sequence and the V_{L} domain sequence in the DOTA binding fragment is any one of SEQ ID NOs: 9-11.

In any and all embodiments of the fusion protein of the present technology, the DOTA binding fragment is located at the N-terminus of the first transmembrane domain or the glycosylphosphatidylinositol (GPI)-anchored polypeptide.

Additionally or alternatively, in some embodiments, the fusion protein of the present technology further comprises a reporter gene. The reporter gene may be a fluorescent reporter gene, a chemiluminescent reporter gene, or a bioluminescent reporter gene and/or may be located at the C-terminus of the first transmembrane domain or the glycosylphosphatidylinositol (GPI)-anchored polypeptide. Examples of suitable fluorescent reporter genes include, but are not limited to, GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, and Dronpa. Examples of bioluminescent reporter genes include, but are not limited to, Aequorin, firefly luciferase, Renilla luciferase, red luciferase, luxAB, and nanoluciferase. Examples of suitable chemiluminescent reporter genes include β-galactosidase, horseradish peroxidase (HRP), and alkaline phosphatase. Peroxidases generate peroxide that oxidizes luminol in a reaction that generates light, whereas alkaline phosphatases remove a phosphate from a substrate molecule, destabilizing it and initiating a cascade that results in the emission of light.

In any of the foregoing embodiments, the fusion protein further comprises a spacer domain interspersed between the DOTA binding fragment and the first transmembrane domain or the glycosylphosphatidylinositol (GPI)-anchored polypeptide. In some embodiments, the spacer domain is a Fc domain or a polyhistidine tag. In certain embodiments, the Fc domain comprises a Fc fragment of a mammalian IgG, e.g., human IgG. In some embodiments, the Fc fragment comprises or consists of the Fc region (e.g., CH2 domain and CH3 domain) of a mammalian IgG, e.g., human IgG. In certain embodiments, the Fc fragment comprises or consists of the Fe region (e.g., CH2 domain and CH3 domain) of human IgG₁, human IgG₂, human IgG₃, or human IgG₄. Exemplary sequences of Fc regions of (e.g., CH2 domain and CI-13 domain) human IgG₁, human IgG₂, human IgG₃, or human IgG₄ are provided below:

Fc region of Human IgG1
(SEQ ID NO: 12)
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK
Fc region of Human IgG2
(SEQ ID NO: 13)
GPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEK
TISKTKGQPREPQVYTLPPSREEMTKNQVSLTCLVKGFYPSDISVEWES
NGQPENNYKTTPPMLDSDGSFFLYSKLTVDKSRWQQGNVFSCSVMHEAL
HNHYTQKSLSLSPGK
Fc region of Human IgG3
(SEQ ID NO: 14)
GPSVFLFPPKPKDTLMISRTPEVTCVVVDVSHEDPEVQFKWYVDGVEVH
NAKTKPREEQYNSTFRVVSVLTVLHQDWLNGKEYKCKVSNKALPAPIEK
TISKTKGQPREPQVYTLPPSREEMTKNQVSLTCLVKGFYPSDIAVEWES
SGQPENNYNTTPPMLDSDGSFFLYSKLTVDKSRWQQGNIFSCSVMHEAL
HNRFTQKSLSLSPGK
Fc region of Human IgG4
(SEQ ID NO: 15)
GPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEAL
HNHYTQKSLSLSLGK
IgG4 CH2-CH3 spacer domain
(SEQ ID NO: 16)
DLEPKSPDKTHTCPPCPAPPVAGPSVFLFPPKPKDTLMISRTPEVTCVV
VDVSHEDPEVKFNWYVDGVEVHNAKTKPREEQYQSTYRVVSVLTVLHQD
WLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKL
TVDKSRWQQGNVFSCSVMHEALHNHYTQKSLSLSPGK

In some embodiments, the Fc fragment comprises or consists of an amino acid sequence having at least 80% (e.g., at least 80%, 85%, 90%, 95%, 97%, 99%, or more) identity to the Fc region (e.g., CH2 domain and CH3 domain) of a human IgG including the amino acid sequence of any one of SEQ ID NOs: 12-16. Additionally or alternatively, the Fc fragment comprises one or more mutations to prevent interaction with FcγRs. Examples of Fc mutations that prevent interaction with FcγRs are described in Hudecek et al., Cancer Immunol Res 3(2): 125-35 (2015).

Examples of glycosylphosphatidylinositol (GPI)-anchored polypeptides include, but are not limited to, uromodulin (Tamm-Horsfall glycoprotein), carbonic anhydrase type IV, alkaline phosphatase, Thy-1, BP-3, aminopeptidase P, and dipeptidylpeptidase.

Additionally or alternatively, in some embodiments, the fusion protein does not include an internal ribosome entry site (IRES), or a 2A self-cleaving peptide.

In accordance with the presently disclosed subject matter, the first transmembrane domain of the fusion protein may comprise an amino acid sequence that is at least 80% (e.g., at least 80%, 85%, 90%, 95%, 97%, 99%, or more) identical to an amino acid sequence of a transmembrane region of CD4 (SEQ ID NO: 17), CD8 (SEQ ID NO: 18), CD28 (SEQ ID NO: 19), CD3ζ (SEQ ID NO: 20) or 4-1BB ligand receptor (SEQ ID NO: 21).

In certain embodiments, the first transmembrane domain comprises an amino acid sequence that is at least 80% (e.g., at least 80%, 85%, 90%, 95%, 97%, 99%, or more) identical to an amino acid sequence of a transmembrane region of CD4, as set forth in SEQ ID NO: 17 as provided below:

(SEQ ID NO: 17)
MALIVLGGVAGLLLFIGLGIFFCVRCRH

In certain embodiments, the first transmembrane domain comprises an amino acid sequence that is at least 80% (e.g., at least 80%, 85%, 90%, 95%, 97%, 99%, or more) identical to an amino acid sequence of a transmembrane region of CD8, as set forth in SEQ ID NO: 18 as provided below:

(SEQ ID NO: 18)
PTTTPAPRPPTPAPTIASQPLSLRPEACRPAAGGAVHTRGLDFACDIYI
WAPLAGTCGVLLLSLVITLYCN

In certain embodiments, the first transmembrane domain comprises an amino acid sequence that is at least 80% (e.g., at least 80%, 85%, 90%, 95%, 97%, 99%, or more) identical to an amino acid sequence of a transmembrane region of CD28, as set forth in SEQ ID NO: 19 as provided below:

(SEQ ID NO: 19)
FWVLVVVGGVLACYSLLVTVAFIIFWV

In certain embodiments, the first transmembrane domain comprises an amino acid sequence that is at least 80% (e.g., at least 80%, 85%, 90%, 95%, 97%, 99%, or more) identical to an amino acid sequence of a transmembrane region of CD3ζ, as set forth in SEQ ID NO: 20 as provided below:

(SEQ ID NO: 20)
LCYLLDGILFIYGVILTALFL

In certain embodiments, the first transmembrane domain comprises an amino acid sequence that is at least 80% (e.g., at least 80%, 85%, 90%, 95%, 97%, 99%, or more) identical to an amino acid sequence of a transmembrane region of 4-1BB ligand receptor, as set forth in SEQ ID NO: 21 as provided below:

(SEQ ID NO: 21)
IISFFLALTSTALLFLLFFLTLRFSVV

Additionally or alternatively, in some embodiments, the fusion protein of the present technology further comprises an endoplasmic reticulum signal sequence. In certain embodiments, the endoplasmic reticulum signal sequence is a CD4 signal peptide comprising the sequence MNRGVPFRHLLLVLQLALLPAATQG (SEQ ID NO: 22). Additionally or alternatively, in some embodiments, the fusion protein of the present technology further comprises a CD8 signal peptide (e.g., MALPVTALLLPLALLLHAARP (SEQ ID NO: 23) or MRPRLWLLLAAQLTVLHGNSV (SEQ ID NO: 24)), a CD28 signal peptide (e.g., MLRLLLALNLFPSIQVTG (SEQ ID NO: 25)), or an IL2 signal peptide (e.g., MYRMQLLSCIALSLALVTNS (SEQ ID NO: 26)).

In any and all embodiments of the fusion protein disclosed herein, the fusion protein further comprises a receptor that binds to a target antigen. The receptor may be a T cell receptor, a native cell receptor, a non-native cell receptor, or a chimeric antigen receptor.

### Chimeric Antigen Receptors

CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

In some embodiments, the fusion proteins of the present technology or the recombinant immune cells provided herein comprise a “first generation” CAR, a “second generation” CAR or a “third generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD3ζ). “Third generation” CARs comprise those that provide multiple co-stimulation (e.g., CD28 and 4-1BB) and activation (e.g., CD3ζ).

In accordance with the presently disclosed subject matter, the CARs of the fusion proteins of the present technology or recombinant immune cells provided herein comprise an extracellular antigen-binding domain, a transmembrane domain and an intracellular domain.

Extracellular Antigen Binding Domain of a CAR. In certain embodiments, the extracellular antigen-binding domain of a CAR specifically binds a tumor antigen. In certain embodiments, the extracellular antigen-binding domain is derived from a monoclonal antibody (mAb) that binds to a tumor antigen. In some embodiments, the extracellular antigen-binding domain comprises an scFv. In some embodiments, the extracellular antigen-binding domain comprises a Fab, which is optionally crosslinked. In a some embodiments, the extracellular binding domain comprises a F(ab)₂. In some embodiments, any of the foregoing molecules are comprised in a fusion protein with a heterologous sequence to form the extracellular antigen-binding domain. In certain embodiments, the extracellular antigen-binding domain comprises a human scFv that binds specifically to a tumor antigen. In certain embodiments, the scFv is identified by screening scFv phage library with tumor antigen-Fc fusion protein.

In certain embodiments, the extracellular antigen-binding domain of a presently disclosed CAR has a high binding specificity and high binding affinity to a tumor antigen (e.g., a mammalian tumor antigen, such as a human tumor antigen). For example, in some embodiments, the extracellular antigen-binding domain of the CAR (embodied, for example, in a human scFv or an analog thereof) binds to a particular tumor antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸M or less, about 1×10⁻⁸M or less, about 5×10⁻⁹ or less, about 4×10⁻⁹ or less, about 3×10⁻⁹ or less, about 2×10⁻⁹ or less, or about 1×10⁻⁹M or less. In certain non-limiting embodiments, the K_{d} is from about 3×10⁻⁹M or less. In certain non-limiting embodiments, the K_{d} is from about 3×10⁻⁹ to about 2×10⁻⁷.

Binding of the extracellular antigen-binding domain (embodiment, for example, in a human scFv or an analog thereof) of a presently disclosed tumor antigen-targeted CAR can be confirmed by, for example, enzyme-linked immunosorbent assay (ELISA), radioimmunoassay (MA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the tumor antigen-targeted CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the human scFv of a presently disclosed tumor antigen-targeted CAR is labeled with GFP.

In some embodiments, the extracellular antigen-binding domain of the expressed CAR binds to tumor antigen that is expressed by a tumor cell. In some embodiments, the extracellular antigen-binding domain of the expressed CAR binds to tumor antigen that is expressed on the surface of a tumor cell. In some embodiments, the extracellular antigen-binding domain of the expressed CAR binds to tumor antigen that is expressed on the surface of a tumor cell in combination with an MHC protein. In some embodiments, the MEW protein is a MHC class I protein. In some embodiments, the MEW Class I protein is an HLA-A, HLA-B, or HLA-C molecules. In some embodiments, the extracellular antigen-binding domain of the expressed CAR binds to tumor antigen that is expressed on the surface of a tumor cell not in combination with an MHC protein.

In some embodiments, the extracellular antigen-binding domain of the expressed CAR binds to tumor antigen selected from among 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the extracellular antigen-binding domain of the expressed CAR binds to tumor antigen selected from among BCMA, CD19, mesothelin, MUC16, PSCA, WT1, and PRAME. Exemplary extracellular antigen-binding domains and methods of generating such domains and associated CARs are described in, e.g., WO2016/191246, WO2017/023859, WO2015/188141, WO2015/070061, WO2012/135854, WO2014/055668, which are incorporated by reference in their entirety, including the sequence listings provided therein.

In some embodiments, the extracellular antigen-binding domain of the expressed CAR binds to a CD19 tumor antigen. In some embodiments, the extracellular antigen-binding domain of the expressed CAR binds to a CD19 tumor antigen presented in the context of an MEW molecule. In some embodiments, the extracellular antigen-binding domain of the expressed CAR binds to a CD19 tumor antigen presented in the context of an HLA-A2 molecule.

In some embodiments, the extracellular antigen-binding domain of the expressed CAR binds to a “preferentially expressed antigen in melanoma” (PRAME) tumor antigen. In some embodiments, the extracellular antigen-binding domain of the expressed CAR binds to a PRAME tumor antigen presented in the context of an MEW molecule. In some embodiments, the extracellular antigen-binding domain of the expressed CAR binds to a PRAME tumor antigen presented in the context of an HLA-A2 molecule.

In some embodiments, extracellular antigen-binding domain of the expressed CAR binds to a WT1 (Wilm's tumor protein 1) tumor antigen. In some embodiments, the extracellular antigen-binding domain of the expressed CAR binds to a WT1 tumor antigen presented in the context of an MEW molecule. In some embodiments, the extracellular antigen-binding domain binds to a WT1 tumor antigen presented in the context of an HLA-A2 molecule.

In some embodiments, extracellular antigen-binding domain of the expressed CAR binds to a MUC16 tumor antigen. In some embodiments, the extracellular antigen-binding domain of the expressed CAR binds to a MUC16 tumor antigen presented in the context of an MEW molecule. In some embodiments, the extracellular antigen-binding domain binds to a MUC16 tumor antigen presented in the context of an HLA-A2 molecule.

In some embodiments, extracellular antigen-binding domain of the expressed CAR binds to a mesothelin tumor antigen. In some embodiments, the extracellular antigen-binding domain of the expressed CAR binds to a mesothelin tumor antigen presented in the context of an MEW molecule. In some embodiments, the extracellular antigen-binding domain binds to a mesothelin tumor antigen presented in the context of an HLA-A2 molecule.

In some embodiments, extracellular antigen-binding domain of the expressed CAR binds to a BCMA (B-cell maturation antigen) tumor antigen. In some embodiments, the extracellular antigen-binding domain of the expressed CAR binds to a BCMA tumor antigen presented in the context of an MHC molecule. In some embodiments, the extracellular antigen-binding domain binds to a BCMA tumor antigen presented in the context of an HLA-A2 molecule.

In some embodiments, extracellular antigen-binding domain of the expressed CAR binds to a PSCA (prostate stem cell antigen) tumor antigen. In some embodiments, the extracellular antigen-binding domain of the expressed CAR binds to a BCMA tumor antigen presented in the context of an MHC molecule. In some embodiments, the extracellular antigen-binding domain binds to a BCMA tumor antigen presented in the context of an HLA-A2 molecule.

In certain embodiments, the extracellular antigen-binding domain (e.g., human scFv) comprises a heavy chain variable region and a light chain variable region, optionally linked with a linker sequence, for example a linker peptide (e.g., SEQ ID NOs: 9-11), between the heavy chain variable region and the light chain variable region. In certain embodiments, the extracellular antigen-binding domain is a human scFv-Fc fusion protein or full length human IgG with V_{H} and V_{L} regions.

In certain embodiments, the extracellular antigen-binding domain comprises a human scFv that binds to a CD19 antigen. In some embodiments, the scFv comprises a polypeptide having an amino acid sequence of SEQ ID NO: 27.

(SEQ ID NO: 27)
EVKLQQSGAELVRPGSSVKISCKASGYAFSSYWMNWVKQRPGQGLEWIG
QIYPGDGDTNYNGKFKGQATLTADKSSSTAYMQLSGLTSEDSAVYFCAR
KTISSVVDFYFDYWGQGTTVTVSSGGGGSGGGGSGGGGSDIELTQSPKF
MSTSVGDRVSVTCKASQNVGTNVAWYQQKPGQSPKPLIYSATYRNSGVP
DRFTGSGSGTDFTLTITNVQSKDLADYFCQQYNRYPYTSGGGTKLEIK
R.

Additionally or alternatively, in some embodiments, the scFv comprises a polypeptide having an amino acid sequence of SEQ ID NO: 28.

(SEQ ID NO: 28)
MALPVTALLLPLALLLHAEVKLQQSGAELVRPGSSVKISCKASGYAFSS
YWMNWVKQRPGQGLEWIGQIYPGDGDTNYNGKFKGQATLTADKSSSTAY
MQLSGLTSEDSAVYFCARKTISSVVDFYFDYWGQGTTVTVSSGGGGSGG
GGSGGGGSDIELTQSPKFMSTSVGDRVSVTCKASQNVGTNVAWYQQKPG
QSPKPLIYSATYRNSGVPDRFTGSGSGTDFTLTITNVQSKDLADYFCQQ
YNRYPYTSGGGTKLEIKR

In some embodiments, the scFv comprises a polypeptide having an amino acid sequence that is at least 80%, at least 85%, at least 90%, or at least 95% identical to SEQ ID NO: 27 or SEQ ID NO: 28. For example, the scFv comprises a polypeptide having an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 27 or SEQ ID NO: 28.

In some embodiments, the scFv is encoded by a nucleic acid having a nucleic acid sequence of SEQ ID NO: 29.

(SEQ ID NO: 29)
GAGGTGAAGCTGCAGCAGTCTGGGGCTGAGCTGGTGAGGCCTGGGTCCT
CAGTGAAGATTTCCTGCAAGGCTTCTGGCTATGCATTCAGTAGCTACTG
GATGAACTGGGTGAAGCAGAGGCCTGGACAGGGTCTTGAGTGGATTGGA
CAGATTTATCCTGGAGATGGTGATACTAACTACAATGGAAAGTTCAAGG
GTCAAGCCACACTGACTGCAGACAAATCCTCCAGCACAGCCTACATGCA
GCTCAGCGGCCTAACATCTGAGGACTCTGCGGTCTATTTCTGTGCAAGA
AAGACCATTAGTTCGGTAGTAGATTTCTACTTTGACTACTGGGGCCAAG
GGACCACGGTCACCGTCTCCTCAGGTGGAGGTGGATCAGGTGGAGGTGG
ATCTGGTGGAGGTGGATCTGACATTGAGCTCACCCAGTCTCCAAAATTC
ATGTCCACATCAGTAGGAGACAGGGTCAGCGTCACCTGCAAGGCCAGTC
AGAATGTGGGTACTAATGTAGCCTGGTATCAACAGAAACCAGGACAATC
TCCTAAACCACTGATTTACTCGGCAACCTACCGGAACAGTGGAGTCCCT
GATCGCTTCACAGGCAGTGGATCTGGGACAGATTTCACTCTCACCATCA
CTAACGTGCAGTCTAAAGACTTGGCAGACTATTTCTGTCAACAATATAA
CAGGTATCCGTACACGTCCGGAGGGGGGACCAAGCTGGAGATCAAACGG
GCGGCCGCA.

In some embodiments, the scFv is encoded by a nucleic acid having a nucleic acid sequence of SEQ ID NO: 30.

(SEQ ID NO: 30)
ATGGCTCTCCCAGTGACTGCCCTACTGCTTCCCCTAGCGCTTCTCCTGC
ATGCAGAGGTGAAGCTGCAGCAGTCTGGGGCTGAGCTGGTGAGGCCTGG
GTCCTCAGTGAAGATTTCCTGCAAGGCTTCTGGCTATGCATTCAGTAGC
TACTGGATGAACTGGGTGAAGCAGAGGCCTGGACAGGGTCTTGAGTGGA
TTGGACAGATTTATCCTGGAGATGGTGATACTAACTACAATGGAAAGTT
CAAGGGTCAAGCCACACTGACTGCAGACAAATCCTCCAGCACAGCCTAC
ATGCAGCTCAGCGGCCTAACATCTGAGGACTCTGCGGTCTATTTCTGTG
CAAGAAAGACCATTAGTTCGGTAGTAGATTTCTACTTTGACTACTGGGG
CCAAGGGACCACGGTCACCGTCTCCTCAGGTGGAGGTGGATCAGGTGGA
GGTGGATCTGGTGGAGGTGGATCTGACATTGAGCTCACCCAGTCTCCAA
AATTCATGTCCACATCAGTAGGAGACAGGGTCAGCGTCACCTGCAAGGC
CAGTCAGAATGTGGGTACTAATGTAGCCTGGTATCAACAGAAACCAGGA
CAATCTCCTAAACCACTGATTTACTCGGCAACCTACCGGAACAGTGGAG
TCCCTGATCGCTTCACAGGCAGTGGATCTGGGACAGATTTCACTCTCAC
CATCACTAACGTGCAGTCTAAAGACTTGGCAGACTATTTCTGTCAACAA
TATAACAGGTATCCGTACACGTCCGGAGGGGGGACCAAGCTGGAGATCA
AACGG

In some embodiments, the scFv is encoded by a nucleic acid having a nucleic acid sequence that is at least 80%, at least 85%, at least 90%, or at least 95% identical to SEQ ID NO: 29 or SEQ ID NO: 30. In some embodiments, the scFv is encoded by a nucleic acid having a nucleic acid sequence of SEQ ID NO: 29 or SEQ ID NO: 30. In some embodiments, the scFv is encoded by a nucleic acid having a nucleic acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 29 or SEQ ID NO: 30.

In certain non-limiting embodiments, an extracellular antigen-binding domain of the presently disclosed CAR can comprise a linker connecting the heavy chain variable region and light chain variable region of the extracellular antigen-binding domain. In certain embodiments, the linker comprises amino acids having the sequence set forth in any one of SEQ ID NOs: 9-11.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway.

In certain embodiments, the signal peptide is covalently joined to the N-terminus of the extracellular antigen-binding domain. In certain embodiments, the signal peptide comprises a CD4 signal peptide (e.g., MNRGVPFRHLLLVLQLALLPAATQG (SEQ ID NO: 22)), a CD8 signal peptide (e.g., MALPVTALLLPLALLLHAARP (SEQ ID NO: 23) or MRPRLWLLLAAQLTVLHGNSV (SEQ ID NO: 24)), a CD28 signal peptide (e.g., MLRLLLALNLFPSIQVTG (SEQ ID NO: 25)), or an IL2 signal peptide (e.g., MYRMQLLSCIALSLALVTNS (SEQ ID NO: 26)).

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the fusion protein of the present technology may include a second transmembrane domain (i.e., the CAR transmembrane domain) that comprises a transmembrane region of CD4 (SEQ ID NO: 17), CD8 (SEQ ID NO: 18), CD28 (SEQ ID NO: 19), CD3ζ (SEQ ID NO: 20) or 4-1BB ligand receptor (SEQ ID NO: 21).

In certain non-limiting embodiments, the fusion protein or recombinant immune cells disclosed herein can also comprise a spacer region that links the extracellular antigen-binding domain of the CAR to the second transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG₁, the CH2CH3 region of immunoglobulin and portions of CD3, a portion of a CD28 polypeptide, a portion of a CD8 polypeptide, a variation of any of the foregoing which is at least about 80%, at least about 85%>, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3 polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170, or fragments thereof, and/or may optionally comprise up to one or up to two or up to three conservative amino acid substitutions. In certain embodiments, the CD3ζ polypeptide can have an amino acid sequence that is a consecutive portion of SEQ ID NO: 31, which is at least 20, or at least 30, or at least 40, or at least 50, and up to 164 amino acids in length. Alternatively or additionally, in various embodiments, the CD3ζ polypeptide has an amino acid sequence of amino acids 1 to 164, 1 to 50, 50 to 100, 100 to 150, or 150 to 164 of SEQ ID NO: 31. In certain embodiments, the CD3ζ polypeptide has an amino acid sequence of amino acids 52 to 164 of SEQ ID NO: 31.

SEQ ID NO: 31 is provided below:

(SEQ ID NO: 31)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the CD3ζ polypeptide has the amino acid sequence set forth in SEQ ID NO: 32, which is provided below:

(SEQ ID NO: 32)
RVKFSRSAEPPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In certain embodiments, the CD3ζ polypeptide has the amino acid sequence set forth in SEQ ID NO: 33, which is provided below:

(SEQ ID NO: 33)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a NCBI Reference No: P41273 or NP_001552 (SEQ ID NO: 34) or fragments thereof, and/or may optionally comprise up to one or up to two or up to three conservative amino acid substitutions.

SEQ ID NO: 34 is provided below:

(SEQ ID NO: 34)
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLGTK
ERDWCGPSPADLSPGASSVTPPAPAREPGHSPQIISFFLALTSTALLFLL
FFLTLRFSWKRGRKKLLYIFKQPFMRPVQTTQEEDGCSCRFPEEEEGGCE
L

In certain embodiments, the 4-1BB co-stimulatory domain has the amino acid sequence set forth in SEQ ID NO: 35, which is provided below:

(SEQ ID NO: 35)
KRGRKKLLYIFKQPFMRPVQTTQEEDGCSCRFPEEEEGGCEL

An OX40 polypeptide can have an amino acid sequence that is at least about 85%, about 90%, about 95%, about 96%, about 97%, about 98%, about 99% or 100% homologous to the sequence having a NCBI Reference No: P43489 or NP_003318 (SEQ ID NO: 36), or fragments thereof, and/or may optionally comprise up to one or up to two or up to three conservative amino acid substitutions.

SEQ ID NO: 36 is provided below:

(SEQ ID NO: 36)
MCVGARRLGRGPCAALLLLGLGLSTVTGLHCVGDTYPSNDRCCHECRPGN
GMVSRCSRSQNTVCRPCGPGFYNDWSSKPCKPCTWCNLRSGSERKQLCTA
TQDTVCRCRAGTQPLDSYKPGVDCAPCPPGHFSPGDNQACKPWTNCTLAG
KHTLQPASNSSDAICEDRDPPATQPQETQGPPARPITVQPTEAWPRTSQG
PSTRPVEVPGGRAVAAILGLGLVLGLLGPLAILLALYLLRRDQRLPPDAH
KPPGGGSFRTPIQEEQADAHSTLAKI

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 37) or fragments thereof, and/or may optionally comprise up to one or up to two or up to three conservative amino acid substitutions.

SEQ ID NO: 37 is provided below:

(SEQ ID NO: 37)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VWCILGCILICWLTKKKYSSSVHDPNGEYMFMRATAKKSRLTDVTL

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif (SEQ ID NO: 47) able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 38) (homology herein may be determined using standard software such as BLAST or FASTA) or fragments thereof, and/or may optionally comprise up to one or up to two or up to three conservative amino acid substitutions.

SEQ ID NO: 38 is provided below:

(SEQ ID NO: 38)
MACLGFQRHKAQLNLATRTWPCTLLFFLLFIPVFCKAMHVAQPAWLASSR
GIASFVCEYASPGKATEVRVTVLRQADSQVTEVCAATYMMGNELTFLDDS
ICTGTSSGNQLTIQGLRAMDTGLYICKVELMYPPPYYLGIGNGTQIYVID
PEPCPDSDFLLWILAAVSSGLFFYSFLLTAVSLSKMLKKRSPLTTGVYVK
MPPTEPECEKQFQPYFIPIN

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system. In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 39) or fragments thereof, and/or may optionally comprise up to one or up to two or up to three conservative amino acid substitutions.

SEQ ID NO: 39 is provided below:

(SEQ ID NO: 39)
MQIPQAPWPVVWAVLQLGWRPGWFLDSPDRPWNPPTFSPALLWTEGDNAT
FTCSFSNTSESFVLNWYRMSPSNQTDKLAAFPEDRSQPGQDCRFRVTQLP
NGRDFHMSVVRARRNDSGTYLCGAISLAPKAQIKESLRAELRVTERRAEV
PTAHPSPSPRPAGQFQTLVVGWGGLLGSLVLLVWVLAVICSRAARGTIGA
RRTGQPLKEDPSAVPVFSVDYGELDFQWREKTPEPPVPCVPEQTEYATIV
FPSGMGTSSPARRGSADGPRSAQPLRPEDGHCSWPL

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 40) or fragments thereof, and/or may optionally comprise up to one or up to two or up to three conservative amino acid substitutions.

SEQ ID NO: 40 is provided below:

(SEQ ID NO: 40)
MWEAQFLGLLFLQPLWVAPVKPLQPGAEVPWWAQEGAPAQLPCSPTIPLQ
DLSLLRRAGVTWQHQPDSGPPAAAPGHPLAPGPHPAAPSSWGPRPRRYTV
LSVGPGGLRSGRLPLQPRVQLDERGRQRGDFSLWLRPARRADAGEYRAAV
HLRDRALSCRLRLRLGQASMTASPPGSLRASDWVILNCSFSRPDRPASVH
WFRNRGQGRVPVRESPHHHLAESFLFLPQVSPMDSGPWGCILTYRDGFNV
SIMYNLTVLGLEPPTPLTVYAGAGSRVGLPCRLPAGVGTRSFLTAKWTPP
GGGPDLLVTGDNGDFTLRLEDVSQAQAGTYTCHIHLQEQQLNATVTLAII
TVTPKSFGSPGSLGKLLCEVTPVSGQERFVWSSLDTPSQRSFSGPWLEAQ
EAQLLSQPWQCQLYQGERLLGAAVYFTELSSPGAQRSGRAPGALPAGHLL
LFLILGVLSLLLLVTGAFGFHLWRRQWRPRRFSALEQGIHPPQAQSKIEE
LEQEPEPEPEPEPEPEPEPEPEQL

Natural Killer Cell Receptor 2B4 (2B4) mediates non-MHC restricted cell killing on NK cells and subsets of T cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 41) or fragments thereof, and/or may optionally comprise up to one or up to two or up to three conservative amino acid substitutions.

SEQ ID NO: 41 is provided below:

(SEQ ID NO: 41)
MLGQWTLILLLLLKVYQGKGCQGSADHWSISGVPLQLQPNSIQTKVDSIA
WKKLLPSQNGFHHILKWENGSLPSNTSNDRFSFIVKNLSLLIKAAQQQDS
GLYCLEVTSISGKVQTATFQVFVFESLLPDKVEKPRLQGQGKILDRGRCQ
VALSCLVSRDGNVSYAWYRGSKLIQTAGNLTYLDEEVDINGTHTYTCNVS
NPVSWESHTLNLTQDCQNAHQEFRFWPFLVIIVILSALFLGTLACFCVWR
RKRKEKQSETSPKEFLTIYEDVKDLKTRRNHEQEQTFPGGGSTIYSMIQS
QSSAPTSQEPAYTLYSLIQPSRKSGSRKRNHSPSFNSTIYEVIGKSQPKA
QNPARLSRKELENFDVYS

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T Cell inhibition via interaction with tumor necrosis family receptors (TNFR), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 42) or fragments thereof, and/or may optionally comprise up to one or up to two or up to three conservative amino acid substitutions.

SEQ ID NO: 42 is provided below:

(SEQ ID NO: 42)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVYASLNHSVIGPNSRLARNVKEAPTEYASICVRS

### Exemplary Fusion Proteins of the Present Technology

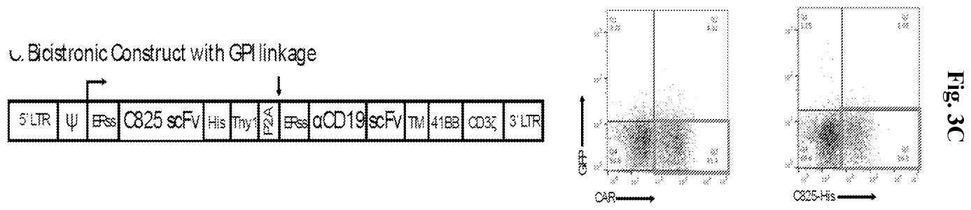

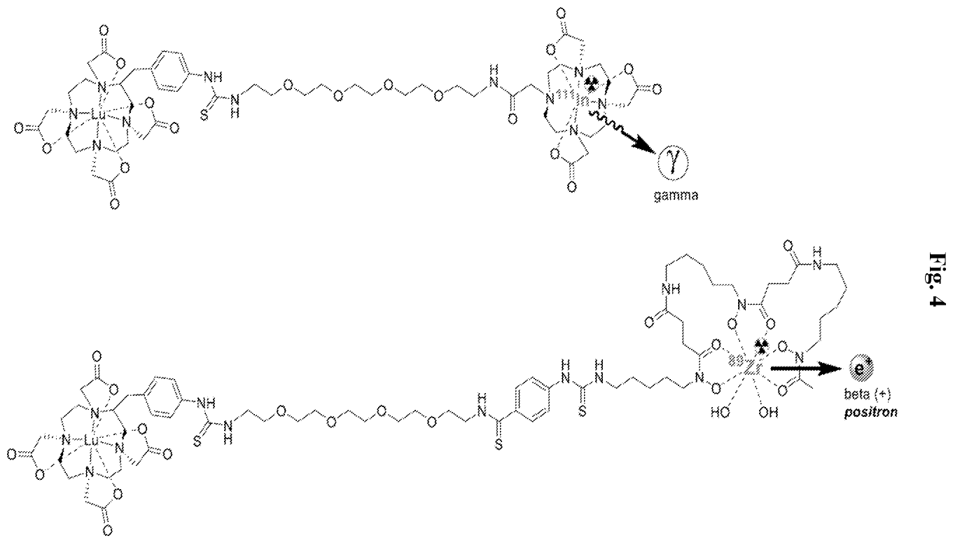

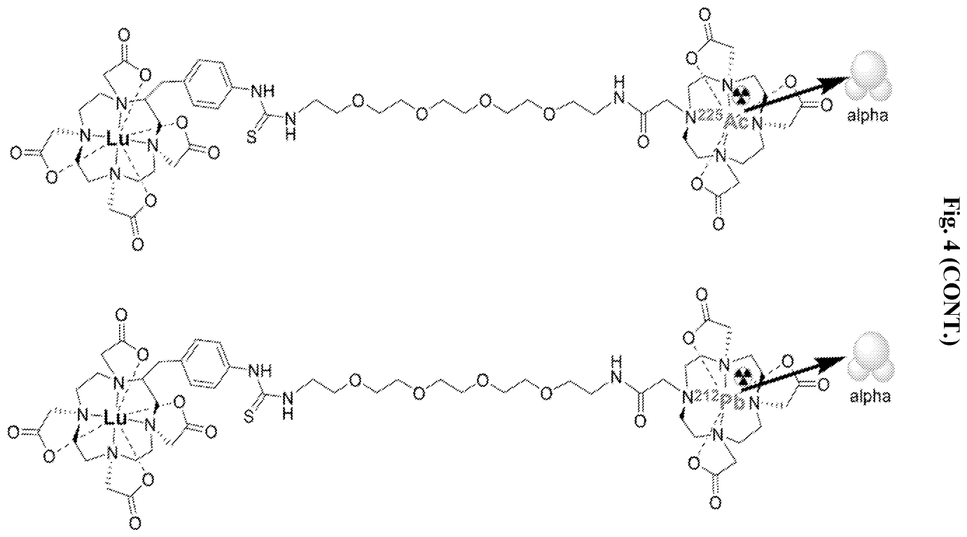

Exemplary fusion proteins of the present technology include those described in FIGS. 3A-3C. In some embodiments, the fusion protein encoding the DOTA binding fragment and the fusion protein encoding the receptor (e.g., CAR) that binds to a target antigen (e.g., tumor antigen) are encoded by separate nucleic acid constructs. The amino acid sequences of the constructs described in FIG. 3A are shown below:

(SEQ ID NO: 43)
TMNRGVPFRHLLLVLQLALLPAATQGHVQLVESGGGLVQPGGSLRLSCAA
SGFSLTDYGVHWVRQAPGKGLEWLGVIWSGGGTAYNTALISRFTISRDNS
KNTLYLQMNSLRAEDTAVYYCARRGSYPYNYFDAWGCGTLVTVSSGGGGS
GGGGSGGGGSQAVVTQEPSLTVSPGGTVTLTCGSSTGAVTASNYANWVQQ
KPGQCPRGLIGGHNNRPPGVPARFSGSLLGGKAALTLLGAQPEDEAEYYC
ALWYSDHWVIGGGTKLTVLGDLEPKSPDKTHTCPPCPAPPVAGPSVFLFP
PKPKDTLMISRTPEVTCVVVDVSHEDPEVKFNWYVDGVEVHNAKTKPREE
QYQSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGKKDPKAAAMALIVLGGVAGLLLFIGLGIFFCVRCRHMVSKGEELFTGV
VPILVELDGDVNGHKFSVSGEGEGDATYGKLTLKFICTTGKLPVPWPTLV
TTLTYGVQCFSRYPDHMKQHDFFKSAMPEGYVQERTIFFKDDGNYKTRAE
VKFEGDTLVNRIELKGIDFKEDGNILGHKLEYNYNSHNVYIMADKQKNGI
KVNFKIRHNIEDGSVQLADHYQQNTPIGDGPVLLPDNHYLSTQSALSKDP
NEKRDHMVLLEFVTAAGITLGMDELYK

*The V_{H} and V_{L} sequences of the C825 scFv are underlined, (G4S)3 linker sequence (SEQ ID NO: 9) is italicized, and first transmembrane domain is in boldface.

19BBz CAR

(SEQ ID NO: 44)
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAPTTTPAPRPPTPAPTIASQPLSLRPEACRPAAGG
AVHTRGLDFACDIYIWAPLAGTCGVLLLSLVITLYCNKRGRKKLLYIFKQ
PFMRPVQTTQEEDGCSCRFPEEEEGGCEL

[symbol]

[symbol]

[symbol]

[symbol]

[symbol]

[symbol]

[symbol]

* The V_{H} and V_{L} sequences of the CD19 scFv are underlined, (G45)3 linker sequence (SEQ ID NO: 9) is italicized, and the second transmembrane domain is in boldface, 41BB is italicized and underlined, and CD3ζ polypeptide is underlined and in boldface.

The amino acid sequences of the fusion proteins described in FIGS. 3B-3C are shown below:

(SEQ ID NO: 45)
TMNRGVPFRHLLLVLQLALLPAATQGHVQLVESGGGLVQPGGSLRLSCAASGFSLT
DYGVHWVRQAPGKGLEWLGVIWSGGGTAYNTALISRFTISRDNSKNTLYLQMNSL
RAEDTAVYYCARRGSYPYNYFDAWGCGTLVTVSSGGGGSGGGGSGGGGSQAVVT
QEPSLTVSPGGTVTLTCGSSTGAVTASNYANWVQQKPGQCPRGLIGGHNNRPPGVP
ARFSGSLLGGKAALTLLGAQPEDEAEYYCALWYSDHWVIGGGTKLTVLGKDPKA
AAMALIVLGGVAGLLLFIGLGIFFCVRCRHMVSKGEELFTGVVPILVELDGDVN
GHKFSVSGEGEGDATYGKLTLKFICTTGKLPVPWPTLVTTLTYGVQCFSRYPDHMK
QHDFFKSAMPEGYVQERTIFFKDDGNYKTRAEVKFEGDTLVNRIELKGIDFKEDGN
ILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYKATNFSLLKQ
AGDVEENPGPALPVTALLLPLALLLHAEVKLQQSGAELVRPGSSVKISCKASGYAFS
SYWMNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGGGSDIE
LTQSPKFMSTSVGDRVSVTCKASQNVGTNVAWYQQKPGQSPKPLIYSATYRNSGV
PDRFTGSGSGTDFTLTITNVQSKDLADYFCQQYNRYPYTSGGGTKLEIKRAAAPTT
TPAPRPPTPAPTIASQPLSLRPEACRPAAGGAVHTRGLDFACDIYIWAPLAGTC
GVLLLSLVITLYCNKRGRKKLLYIFKQPFMRPVQTTQEEDGCSCRFPEEEEGGCEL

[symbol]

[symbol]

[symbol]

[symbol]

[symbol]

[symbol]

[symbol]

[symbol]

(SEQ ID NO: 46)
TMNRGVPFRHLLLVLQLALLPAATQGHVQLVESGGGLVQPGGSLRLSCAASGFSLT
DYGVHWVRQAPGKGLEWLGVIWSGGGTAYNTALISRFTISRDNSKNTLYLQMNSL
RAEDTAVYYCARRGSYPYNYFDAWGCGTLVTVSSGGGGSGGGGSGGGGSQAVVT
QEPSLTVSPGGTVTLTCGSSTGAVTASNYANWVQQKPGQCPRGLIGGHNNRPPGVP
ARFSGSLLGGKAALTLLGAQPEDEAEYYCALWYSDHWVIGGGTKLTVLGHHHHH
HDKLVKCEGISLLAQNTSWLLLLLLSLSLLQATDFMSLATNFSLLKQAGDVEENPG
PMALPVTALLLPLALLLHAEVKLQQSGAELVRPGSSVKISCKASGYAFSSYWMNW
VKQRPGQGLEWIGQIYPGDGDTNYNGKFKGQATLTADKSSSTAYMQLSGLTSEDS
AVYFCARKTISSVVDFYFDYWGQGTTVTVSSGGGGSGGGGSGGGGSDIELTQSPKF
MSTSVGDRVSVTCKASQNVGTNVAWYQQKPGQSPKPLIYSATYRNSGVPDRFTGS
GSGTDFTLTITNVQSKDLADYFCQQYNRYPYTSGGGTKLEIKRAAAPTTTPAPRPP
TPAPTIASQPLSLRPEACRPAAGGAVHTRGLDFACDIYIWAPLAGTCGVLLLSL
VITLYCNKRGRKKLLYIFKQPFMRPVQTTQEEDGCSCRFPEEEEGGCEL

[symbol]

[symbol]

[symbol]

[symbol]

[symbol]

[symbol]

[symbol]

*The V_{H} and V_{L} sequences of the C825 scFv and CD19 scFv are underlined, (G45)3 linker sequence (SEQ ID NO: 9) is italicized, the transmembrane domains are in boldface, 41BB is italicized and underlined, and CD3ζ polypeptide is underlined and in boldface.

### Exemplary Nucleic Acid Constructs of the Present Technology

In one aspect, the present disclosure provides a recombinant nucleic acid sequence encoding any and all embodiments of the fusion proteins disclosed herein. In some embodiments, the recombinant nucleic acid sequence encodes the fusion protein of any one of SEQ ID NOs: 43-46.

In certain embodiments, the receptor that binds to a target antigen (e.g., CAR) and the DOTA binding fragment are expressed as single polypeptide linked by a self-cleaving linker, such as a P2A linker. In certain embodiments, the receptor that binds to a target antigen (e.g., CAR) and the DOTA binding fragment are expressed as two separate polypeptides (See, e.g., FIG. 3A).

In certain embodiments, the CAR comprises an extracellular antigen-binding region that comprises a human scFv that specifically binds to a human tumor antigen, a transmembrane domain comprising a CD28 polypeptide and/or a CD8 polypeptide, and an intracellular domain comprising a CD3ζ polypeptide and a co-stimulatory signaling region that comprises a 4-1BB polypeptide. The CAR also comprises a signal peptide or a leader covalently joined to the N-terminus of the extracellular antigen-binding domain. The signal peptide may include a CD4 signal peptide (e.g., MNRGVPFRHLLLVLQLALLPAATQG (SEQ ID NO: 22)), a CD8 signal peptide (e.g., MALPVTALLLPLALLLHAARP (SEQ ID NO: 23) or MRPRLWLLLAAQLTVLHGNSV (SEQ ID NO: 24)), a CD28 signal peptide (e.g., MLRLLLALNLFPSIQVTG (SEQ ID NO: 25)), or an IL2 signal peptide (e.g., MYRMQLLSCIALSLALVTNS (SEQ ID NO: 26)). In certain embodiments, the human scFv targets a tumor antigen (e.g., 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1).

In some embodiments, the nucleic acid encoding the CAR and the DOTA binding fragment is operably linked to an inducible promoter. In some embodiments, the nucleic acid encoding the CAR and the DOTA binding fragment is operably linked to a constitutive promoter. In some embodiments, the nucleic acid encoding the CAR and the nucleic acid encoding the DOTA binding fragment are operably linked to two separate promoters. In some embodiments, the nucleic acid encoding the CAR is operably linked to a constitutive promoter and the DOTA binding fragment is operably linked to a constitutive promoter. In some embodiments, the nucleic acid encoding the CAR is operably linked to a constitutive promoter and the DOTA binding fragment is operably linked to an inducible promoter.

In some embodiments, the inducible promoter is a synthetic Notch promoter that is activatable in a CAR T cell, where the intracellular domain of the CAR contains a transcriptional regulator that is released from the membrane when engagement of the CAR with the tumor antigen induces intramembrane proteolysis (see, e.g., Morsut et al., Cell 164(4): 780-791 (2016). Accordingly, transcription of the DOTA binding fragment is induced upon binding of the recombinant immune cell with the tumor antigen.

The presently disclosed subject matter also provides isolated nucleic acid molecules encoding the CAR/DOTA binding fragment constructs described herein or a functional portion thereof. In certain embodiments, the isolated nucleic acid molecule encodes an anti-CD19-targeted CAR comprising a human scFv that specifically binds to a human CD19 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a DOTA binding fragment provided herein.

In certain embodiments, the isolated nucleic acid molecule encodes an anti-CD19-targeted CAR comprising a human scFv that specifically binds to a human CD19 polypeptide fused to a synthetic Notch transmembrane domain and an intracellular cleavable transcription factor. In certain embodiments, the isolated nucleic acid molecule encodes a DOTA binding fragment inducible by release of the transcription factor of a synthetic Notch system.

In certain embodiments, the isolated nucleic acid molecule encodes an anti-MUC16-targeted CAR comprising a human scFv that specifically binds to a human MUC16 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a DOTA binding fragment provided herein.

In certain embodiments, the isolated nucleic acid molecule encodes an anti-mesothelin-targeted CAR comprising a human scFv that specifically binds to a human mesothelin polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a DOTA binding fragment provided herein.

In certain embodiments, the isolated nucleic acid molecule encodes an anti-WT1-targeted CAR comprising a human scFv that specifically binds to a human WT1 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a DOTA binding fragment provided herein.

In certain embodiments, the isolated nucleic acid molecule encodes an anti-PSCA-targeted CAR comprising a human scFv that specifically binds to a human PSCA polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a DOTA binding fragment provided herein.

In certain embodiments, the isolated nucleic acid molecule encodes an anti-BCMA-targeted CAR comprising a human scFv that specifically binds to a human BCMA polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and a DOTA binding fragment provided herein.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the targeted CAR (the parent CAR). For example, functional portions encompass the portions, parts or fragments of a tumor antigen-targeted CAR that retains the ability to recognize a target cell, to treat a disease, e.g., solid tumor, to a similar, same, or even a higher extent as the parent CAR. In certain embodiments, an isolated nucleic acid molecule encoding a functional portion of a tumor antigen-targeted CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

### Exemplary Expression Vectors

In another aspect, the present disclosure provides an expression vector comprising any and all embodiments of the recombinant nucleic acid sequences disclosed herein. In certain embodiments, the expression vector comprises the recombinant nucleic acid sequence encoding the fusion protein of any one of SEQ ID NOs: 43-46.

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 48) or a myc tag, hemagglutinin tag or a tag for purification, for example, a GST fusion, and a sequence for directing protein secretion and/or membrane association.

Expression of the antibodies or antigen-binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the antibody chain and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of recombinant immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the tumor antigen-targeted CAR and the DOTA binding fragment can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide tumor antigen-targeted CAR and the DOTA binding fragment expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secretes a cytokine (e.g., 4-1BBL and/or IL-12) in a recombinant immune cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed tumor antigen-targeted CAR is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Immune Cells

In yet another aspect, the present disclosure provides a recombinant immune cell comprising any and all embodiments of the expression vectors described herein.

The presently disclosed subject matter provides recombinant immune cells expressing a DOTA binding fragment and a T-cell receptor (e.g., a CAR) or other ligand that comprises an extracellular antigen-binding domain, a transmembrane domain and an intracellular domain, where the extracellular antigen-binding domain specifically binds tumor antigen, including a tumor receptor or ligand, as described above. In certain embodiments immune cells can be transduced with a presently disclosed CAR/DOTA binding fragment constructs such that the cells express the CAR and the DOTA binding fragment. The presently disclosed subject matter also provides methods of using such cells for the treatment of a tumor. The recombinant immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The recombinant immune cells of the presently disclosed subject matter can express an extracellular antigen-binding domain (e.g., a human scFv, a Fab that is optionally crosslinked, or a F(ab)₂) that specifically binds to a tumor antigen, for the treatment of cancer, e.g., for treatment of solid tumor. Such recombinant immune cells can be administered to a subject (e.g., a human subject) in need thereof for the treatment of cancer. In some embodiments, the immune cell is a lymphocyte, such as a T cell, a B cell or a natural killer (NK) cell. In certain embodiments, the recombinant immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

A presently disclosed recombinant immune cells can further include at least one recombinant or exogenous co-stimulatory ligand. For example, a presently disclosed recombinant immune cells can be further transduced with at least one co-stimulatory ligand, such that the recombinant immune cells co-expresses or is induced to co-express the tumor antigen-targeted CAR and the at least one co-stimulatory ligand. The interaction between the tumor antigen-targeted CAR and at least one co-stimulatory ligand provides a non-antigen-specific signal important for full activation of an immune cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-a, CD134L/OX40L/CD252, CD27L/CD70, Fas ligand (FasL), CD30L/CD153, tumor necrosis factor beta (TNFP)/lymphotoxin-alpha (LT-α), lymphotoxin-beta (LT-β), CD257/B cell-activating factor (BAFF)/BLYS/THANK/TALL-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, or PD-L1/(B7-H1) that are ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the recombinant immune cell comprises one recombinant co-stimulatory ligand that is 4-1BBL. In certain embodiments, the recombinant immune cell comprises two recombinant co-stimulatory ligands that are 4-1BBL and CD80. CARs comprising at least one co-stimulatory ligand are described in U.S. Pat. No. 8,389,282, which is incorporated by reference in its entirety.

Furthermore, a presently disclosed recombinant immune cells can further comprise at least one exogenous cytokine. For example, a presently disclosed recombinant immune cell can be further transduced with at least one cytokine, such that the recombinant immune cells secretes the at least one cytokine as well as expresses the tumor antigen-targeted CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The recombinant immune cells can be generated from peripheral donor lymphocytes, e.g., those disclosed in Sadelain, M., et al., Nat Rev Cancer 3:35-45 (2003) (disclosing peripheral donor lymphocytes genetically modified to express CARs), in Morgan, R. A. et al., Science 314: 126-129 (2006) (disclosing peripheral donor lymphocytes genetically modified to express a full-length tumor antigen-recognizing T cell receptor complex comprising the α and β heterodimer), in Panelli et al., J Immunol 164:495-504 (2000); Panelli et al., J Immunol 164:4382-4392 (2000) (disclosing lymphocyte cultures derived from tumor infiltrating lymphocytes (TILs) in tumor biopsies), and in Dupont et al., Cancer Res 65:5417-5427 (2005); Papanicolaou et al., Blood 102:2498-2505 (2003) (disclosing selectively inv/Yro-expanded antigen-specific peripheral blood leukocytes employing artificial antigen-presenting cells (AAPCs) or pulsed dendritic cells). The recombinant immune cells (e.g., T cells) can be autologous, non-autologous (e.g., allogeneic), or derived in vitro from engineered progenitor or stem cells.

In certain embodiments, a presently disclosed recombinant immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a presently disclosed tumor antigen-targeted CAR and/or DOTA binding fragment.

For example, the higher the CAR expression level in a recombinant immune cell, the greater cytotoxicity and cytokine production the recombinant immune cell exhibits. A recombinant immune cell (e.g., T cell) having a high tumor antigen-targeted CAR expression level can induce antigen-specific cytokine production or secretion and/or exhibit cytotoxicity to a tissue or a cell having a low expression level of tumor antigen-targeted CAR, e.g., about 2,000 or less, about 1,000 or less, about 900 or less, about 800 or less, about 700 or less, about 600 or less, about 500 or less, about 400 or less, about 300 or less, about 200 or less, about 100 or less of tumor antigen binding sites/cell. Additionally or alternatively, the cytotoxicity and cytokine production of a presently disclosed recombinant immune cell (e.g., T cell) are proportional to the expression level of tumor antigen in a target tissue or a target cell. For example, the higher the expression level of human tumor antigen in the target, the greater cytotoxicity and cytokine production the recombinant immune cell exhibits.

The unpurified source of immune cells may be any source known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Suitably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Usually, the cells are collected in a medium comprising 2% fetal calf serum (FCS) or 0.2% bovine serum albumin (BSA) or any other suitable (e.g., sterile), isotonic medium.

In some embodiments, the recombinant immune cells comprise one or more additional modifications. For example, in some embodiments, the recombinant immune cells comprise and express (is transduced to express) an antigen recognizing receptor that binds to a second antigen that is different than selected tumor antigen. The inclusion of an antigen recognizing receptor in addition to a presently disclosed CAR on the recombinant immune cell can increase the avidity of the CAR or the recombinant immune cell comprising thereof on a targeted cell, especially, the CAR is one that has a low binding affinity to a particular tumor antigen, e.g., a K_{d} of about 2×10⁻⁸ M or more, about 5×10⁻⁸ M or more, about 8×10⁻⁸M or more, about 9×10⁻⁸M or more, about 1×10⁻⁷ M or more, about 2×10⁻⁷M or more, or about 5×10⁻⁷M or more.

In certain embodiments, the antigen recognizing receptor is a chimeric co-stimulatory receptor (CCR). CCR is described in Krause, et al., J. Exp. Med. 188(4):619-626(1998), and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology 31(1):71-75 (2013)). With this approach, T-cell activation requires CAR-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises an extracellular antigen-binding domain that binds to an antigen different than selected tumor antigen, a transmembrane domain, and a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second antigen is selected so that expression of both selected tumor antigen and the second antigen is restricted to the targeted cells (e.g., cancerous tissue or cancerous cells). Similar to a CAR, the extracellular antigen-binding domain can be a scFv, a Fab, a F(ab)₂; or a fusion protein with a heterologous sequence to form the extracellular antigen-binding domain. In certain embodiments, the CCR comprises a scFv that binds to CD138, transmembrane domain comprising a CD28 polypeptide, and a co-stimulatory signaling region comprising two co-stimulatory signaling molecules that are CD28 and 4-1BB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells, e.g., myeloma cells. The truncated CAR functions as an adhesion molecule that enhances the avidity of a presently disclosed CAR, especially, one that has a low binding affinity to tumor antigen, thereby improving the efficacy of the presently disclosed CAR or recombinant immune cell (e.g., T cell) comprising thereof. In certain embodiments, the truncated CAR comprises an extracellular antigen-binding domain that binds to CD138, a transmembrane domain comprising a CD8 polypeptide. A presently disclosed T cell comprises or is transduced to express a presently disclosed CAR targeting tumor antigen and a truncated CAR targeting CD138. In certain embodiments, the targeted cells are solid tumor cells. In some embodiments, the recombinant immune cells are further modified to suppress expression of one or more genes. In some embodiments, the recombinant immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the recombinant immune cells are modified to disrupt or reduce expression of an endogenous T-cell receptor gene (see, e.g., WO 2014153470, which is incorporated by reference in its entirety). In some embodiments, the recombinant immune cells are modified to result in disruption or inhibition of PD1, PDL-1 or CTLA-4 (see, e.g., U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Methods for In Vivo Monitoring of Immune Cells in Patients Undergoing Cellular Immunotherapy

In one aspect, the present disclosure provides a method for tracking recombinant immune cells in a subject in vivo comprising: (a) administering to the subject an effective amount of any recombinant immune cell described herein, wherein the recombinant immune cell is configured to localize to a tissue expressing the target antigen recognized by the recombinant immune cell; (b) administering to the subject an effective amount of a DOTA-bearing bischelate, wherein the DOTA-bearing bischelate is configured to bind to the fusion protein expressed by the recombinant immune cell and comprises a radionuclide; and (c) determining the biodistribution of the recombinant immune cells in the subject by detecting radioactive levels emitted by the DOTA-bearing bischelate that are higher than a reference value. In another aspect, the present disclosure provides a method for tracking recombinant immune cells in a subject in vivo comprising: (a) administering to the subject an effective amount of a complex comprising any recombinant immune cell described herein and a DOTA-bearing bischelate comprising a radionuclide, wherein the complex is configured to localize to a tissue expressing the target antigen recognized by the recombinant immune cell; and (b) determining the biodistribution of recombinant immune cells in the subject by detecting radioactive levels emitted by the DOTA-bearing bischelate that are higher than a reference value.

In yet another aspect, the present disclosure provides a method for monitoring viability of recombinant immune cells in a subject comprising: (a) administering to the subject an effective amount of any recombinant immune cell described herein, wherein the recombinant immune cell is configured to localize to a tissue expressing the target antigen recognized by the recombinant immune cell; (b) administering to the subject an effective amount of a DOTA-bearing bischelate, wherein the DOTA-bearing bischelate is configured to bind to the fusion protein expressed by the recombinant immune cell and comprises a radionuclide; (c) detecting radioactive levels emitted by the DOTA-bearing bischelate that are higher than a reference value at a first time point; (d) detecting radioactive levels emitted by the DOTA-bearing bischelate that are higher than a reference value at a second time point; and (e) determining that the recombinant immune cells in the subject are viable when the radioactive levels emitted by the DOTA-bearing bischelate at the second time point are comparable to that observed at the first time point. In some embodiments, the method further comprises administering to the subject a second effective amount of the DOTA-bearing bischelate prior to step (d). Also disclosed herein is a method for monitoring viability of recombinant immune cells in a subject comprising: (a) administering to the subject an effective amount of a complex comprising any recombinant immune cell described herein and a DOTA-bearing bischelate comprising a radionuclide, wherein the complex is configured to localize to a tissue expressing the target antigen recognized by the recombinant immune cell; (b) detecting radioactive levels emitted by the DOTA-bearing bischelate that are higher than a reference value at a first time point; (c) detecting radioactive levels emitted by the DOTA-bearing bischelate that are higher than a reference value at a second time point; and (d) determining that the recombinant immune cells in the subject are viable when the radioactive levels emitted by the DOTA-bearing bischelate at the second time point are comparable to that observed at the first time point.

In yet another aspect, the present disclosure provides a method for monitoring expansion of recombinant immune cells in a subject comprising: (a) administering to the subject an effective amount of any recombinant immune cell described herein, wherein the recombinant immune cell is configured to localize to a tissue expressing the target antigen recognized by the recombinant immune cell; (b) administering to the subject a first effective amount of a DOTA-bearing bischelate, wherein the DOTA-bearing bischelate is configured to bind to the fusion protein expressed by the recombinant immune cell and comprises a radionuclide; (c) detecting radioactive levels emitted by the DOTA-bearing bischelate that are higher than a reference value at a first time point; (d) administering to the subject a second effective amount of the DOTA-bearing bischelate after step (c); (e) detecting radioactive levels emitted by the DOTA-bearing bischelate that are higher than a reference value at a second time point; and (f) determining that the recombinant immune cells in the subject have expanded when the radioactive levels emitted by the DOTA-bearing bischelate at the second time point are higher relative to that observed at the first time point.

In any and all embodiments of the methods disclosed herein, the radioactive levels emitted by the complex or the DOTA-bearing bischelate are detected using positron emission tomography (PET) or single photon emission computed tomography (SPECT).

The DOTA-bearing bischelate may be administered at any time between 1 minute to 4 or more days following administration of the recombinant immune cells expressing any of the fusion proteins disclosed herein. For example, in some embodiments, the DOTA-bearing bischelate is administered 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, or any range therein, following administration of the recombinant immune cells expressing any of the fusion proteins disclosed herein. Alternatively, the DOTA-bearing bischelate may be administered at any time after 4 or more days following administration of the recombinant immune cells expressing any of the fusion proteins disclosed herein.

Additionally or alternatively, in some embodiments of the methods disclosed herein, the DOTA-bearing bischelate is administered intravenously, intramuscularly, intraarterially, intrathecally, intracranially, intracapsularly, intraorbitally, intradermally, intraperitoneally, transtracheally, subcutaneously, intracerebroventricularly, orally, intratumorally, or intranasally. In certain embodiments, the DOTA-bearing bischelate is administered into the cerebral spinal fluid or blood of the subject.

In some embodiments of the methods disclosed herein, the radioactive levels emitted by the DOTA-bearing bischelate are detected between 2 to 120 hours after the DOTA-bearing bischelate is administered. In certain embodiments of the methods disclosed herein, the radioactive levels emitted by the DOTA-bearing bischelate are expressed as the percentage injected dose per gram tissue (% ID/g). The reference value may be calculated by measuring the radioactive levels present in normal tissues, and computing the average radioactive levels present in normal tissues±standard deviation. In some embodiments, the reference value is the standard uptake value (SUV). See Thie JA, J Nucl Med. 45(9):1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

In any and all embodiments of the methods disclosed herein, the radionuclide is an alpha particle-emitting isotope, a beta particle-emitting isotope, or an Auger-emitter. Examples of radionuclides include ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu. In any of the preceding embodiments of the methods disclosed herein, the subject is human.

In any and all embodiments of the methods disclosed herein, the subject is diagnosed with, or is suspected of having cancer. Examples of cancer include, but are not limited to, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof

### EXAMPLES

The present technology is further illustrated by the following Examples, which should not be construed as limiting in any way.

### Example 1: Materials and Methods

Cell lines. 293T cells were used to establish quantitative correlates between cell-membrane expression of C825 and in vivo radiohapten capture efficiency. The 293T cell line is a readily transducible cell line of embryonal renal origin that grows readily both in tissue culture and as solid tumor in vivo in immunologically deficient mice (see below).

Initially, 293T cells were transduced with C825 or subjected to a “mock” transduction and used in a series of in vitro and in vivo experiments designed to evaluate companion biomarker radiohaptens for image-guided targeted radiotherapy during anti-tumor/C825 CAR T-cell infusion.

The 293T cell line was used as a test system to compare the effectiveness of transduction vectors for producing C825 membrane expression. It has been documented that 293T cells exhibit a cancer stem cell-like phenotype when cultured as 3D spheres, as well as form phenotypically stable xenografts with histological appearance of high-grade, poorly differentiated tumor (Debeb et al., Mol Cancer. 9:180 (2010)). 293T cells were used as a comparator for all the tested DOTA-based radiohaptens, to verify in vivo pharmacology of radiohapten binding (uptake and retention) to a membrane-anchored radiohapten antibody, and to verify rapid renal clearance of non-tumor-bound radiohaptens.

Determination of in vitro uptake of reporter probes and assessment of binding kinetics and quantitation of binding sites. A total of 500,000 293T-C825 cells were suspended in RPMI+10% fetal calf serum and incubated at 37° C. for one hour with ¹¹¹In-radiohapten (0.00008-8 nM) (total volume of 300 μL). Cells were harvested with a cell harvester and counted in a WIZARD automatic γ-counter (PerkinElmer) and labeling efficiency (% LE; i.e., radioactivity bound to the cells) was calculated. Activity standards were also harvested each time to correct for non-specific binding. Standard saturation binding parameters were obtained using GraphPad Prism.

Animal xenograft model. 293T-C825 cells (3×10⁶) were injected subcutaneously (s.c.) over the left shoulder and wild-type 293T cells (3×10⁶) over the right shoulder into nude mice (6-8 week old athymic nude-Foxn1^{nu} from Envigo). Seven days later, the mice received intravenous (i.v.) radiotracer administration followed by PET imaging studies.

Small-animal PET/CT imaging. Small-animal PET/CT scans were performed using the Inveon PET/CT system (Siemens). Mice were anesthetized using 1.5-2% isoflurane (Baxter Healthcare) and IV injected with [⁸⁶Y]DOTA-benzene (7.4 MBq). At 5 min, 1 h, 18 h, and 22 h post-injection, mice underwent a 30-min static scans. Data were corrected for decay and detector dead-time, and images were reconstructed by 2D OSEM into 128×128 matrix (0.78×0.78×0.80 mm voxel dimensions). Image counts per voxel per second were converted to activity concentrations (Bq/mL) using a system-specific calibration factor. CT scans were reconstructed using a modified Feldkamp cone beam reconstruction algorithm to generate 512×512×768 voxel image volumes (0.197×0.197×0.197 mm voxel dimensions).

Affinity measurement of DOTA-PEG6 Biotin by Surface Plasmon Resonance (Biacore). sBiacore T-100 Biosensor, streptavidin (SA) sensor chip, and related reagents were purchased from GE Healthcare. The SA chip coated with DOTA-PEG6 Biotin and then flowed over with an antitumor/anti-DOTA IgG-scFv. The KDs were generated from a sensorgram data series consisting of 6 concentrations of anti-tumor/anti-DOTA IgG-scFv, by fitting to a 1:1 binding model using the Biacore T-100 evaluation software. The values are as follows: ka (1/Ms)=7.36E+05; kd (1/s)=5.05E-05; KD (M)=6.86E-11; t½ (s)=13719.6; Chi2 (RU2)=1.03.

Flow Cytometry. Data was collected using a Guava easyCyte HT Flow Cytometer (Millipore Sigma, Jaffrey, NH) or a BD LSR Fortessa (Becton, Dickinson, and Company, Franklin Lakes, NJ). Data was analyzed using Flowjo v10.4 software (Flowjo, Ashland, OR). 293T cells were analyzed with APC conjugated anti-human Fc (Jackson ImmunoResearch, West Grove, PA) or with DOTA-PEG6 Biotin to assess huC825-expression and DOTA-radiohapten capture, respectively. Specifically, for assay of DOTA-radiohapten capture, cells were labeled with DOTA-PEG6 Biotin and subsequently analyzed with Alexa-647 conjugated streptavidin.

DOTA-PEG6 Biotin Synthesis.

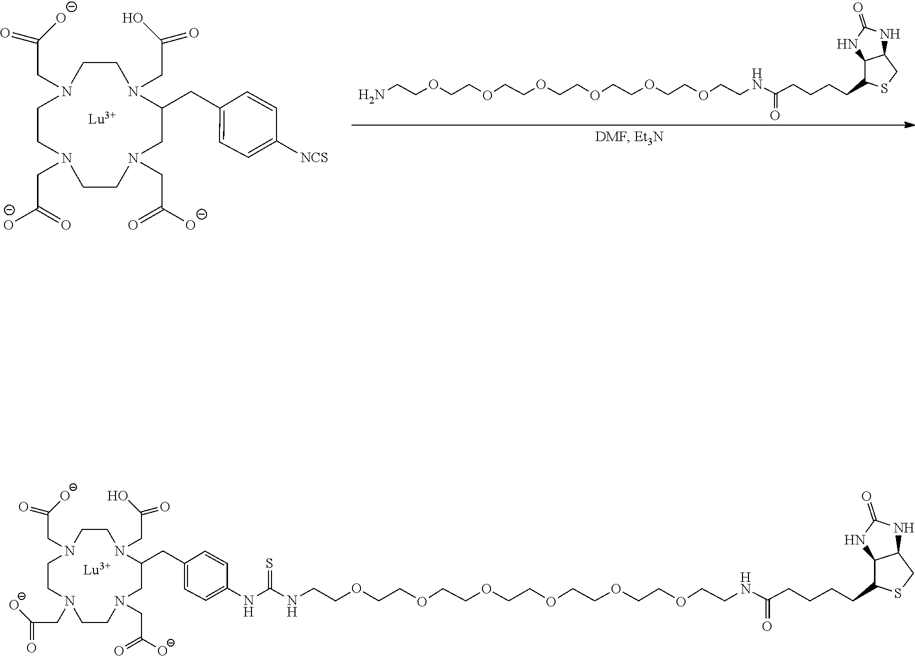

High purity solvents and reagents were purchased from commercial sources and were used without further purification. Proton- and carbon-13 NMR spectra were recorded on a Bruker Avance-600 spectrometer in CDCl₃. All liquid chromatography mass spectrometry (LCMS) data was obtained with a Waters Autopure system comprised of Sample Manager, binary Gradient Module, System Fluidics Organizer, Evaporative Light Scattering Detector, Photodiode Array Detector, 3100 Mass Detector. Binary solvent system: solvent A, 0.05% TFA in water; solvent B, 0.05% TFA in acetonitrile. Analytical method (unless otherwise noted): 5-95% solvent B in 10 min, 1.2 mL/min flow rate. Analytical columns: Waters XBridge, BEH300, C18, 5 μm, 4.6×50 mm. Preparative method: 5-95% solvent B in 30 min, 20 mL/min flow rate. Preparative Columns: Waters XBridge Prep C18, 5 μm, OBD, 19×150 mm.

p-SCN-Bn-DOTA·Lu3⁺ complex: LuCl₃·6 H₂O (142 mg, 365 μmol) was added to 0.6 mL NaOAc (0.4 M solution), then p-SCN-Bn-DOTA·2.5 HCl·2.5 H₂O (50 mg, 73 μmol) was added to the solution. The mixture was stirred at room temperature overnight. Purification was performed by C-18 reverse phase column, using the gradient 0-40% ACN/water. Major isomer 31.22 mg (60.5%) and minor isomer 8 mg (15.2%) were collected.

Dota-biotin-PEG-6-Lu3+: p-SCN-Bn-DOTA·Lu3⁺ complex major isomer (20 mg, 27.6 μmol) and Biotin-PEG-6-NH₂ (15 mg, 27.2 μmol) were added to DMF (0.4 mL), followed by Et₃N (10 μL). The mixture was stirred at room temperature for 4 h. Solvents were removed by evaporation, and purification by HPLC provided 28.34 mg (82%) of the desired product. ¹HNMR (500 MHz, D₂O): δ=7.24-7.20 (m, 4H), 4.51 (dd, 1H, J=8.0 Hz, J=5.0 Hz), 4.33 (d, 1H, J=8.0 Hz, J=5.0 Hz), 3.63-3.21 (m, 40H), 3.03-2.41 (m, 16H), 2.19-2.16 (m, 2H), 1.65-1.50 (m, 4H), 1.34-1.32 (m, 2H). LCMS: calcd: 1274.4 [M+H]⁺. found: 1274.8.

### Example 1A: Synthesis of DOTA·Lu³⁺-PEG 4-DFO

Scheme 1 provides a synthetic route to provide DOTA·Lu³⁺-PEG 4-DFO of the present technology. Experimental details of the synthesis are provided thereafter.

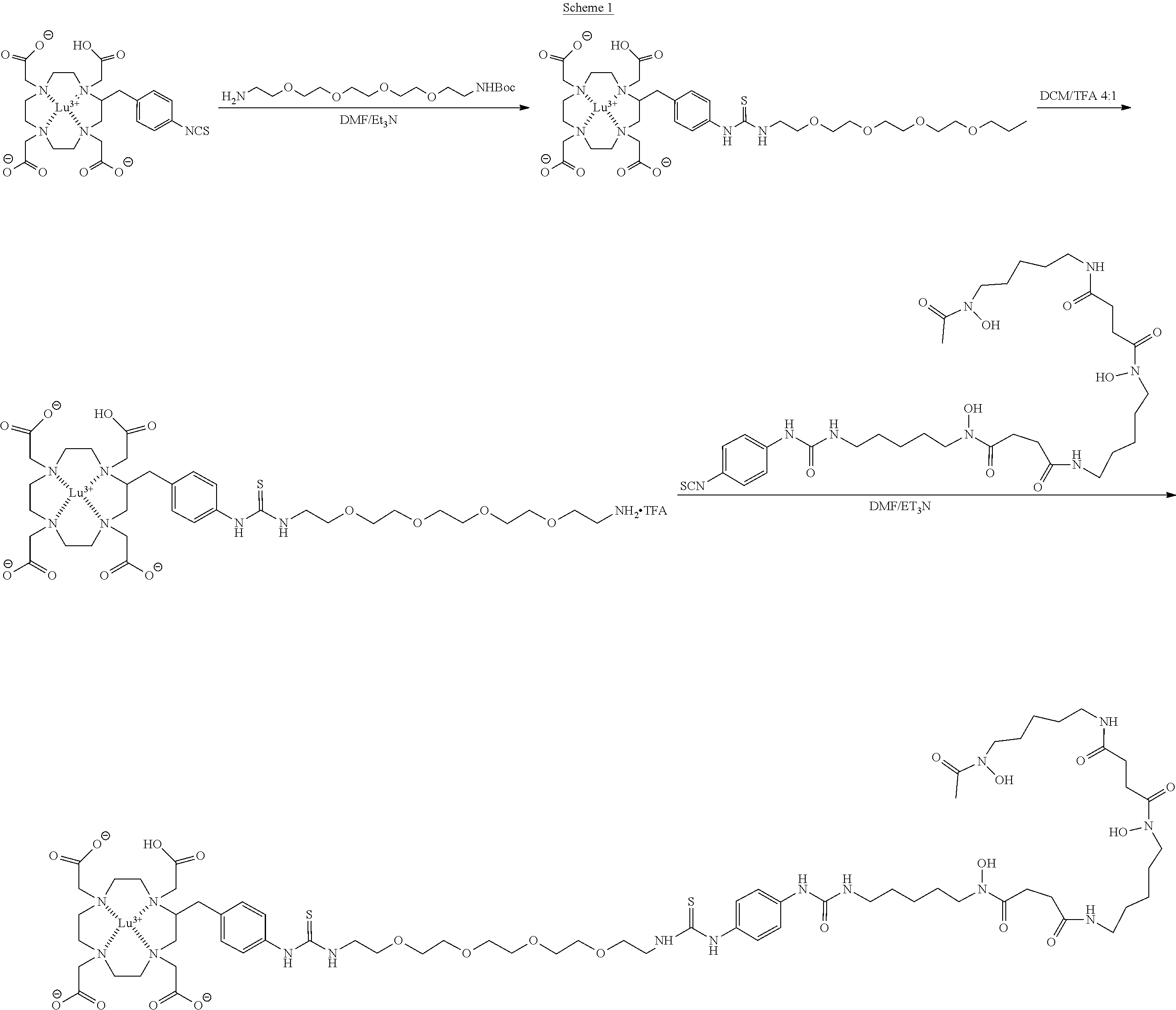

DOTA-Lu³⁺-PEG 4-NHBoc

p-SCN-Bn-DOTA·Lu³⁺ complex (major isomer of Example 1) (30 mg, 41.5 μmol) and Boc-NH-PEG 4-NH₂ (17 mg, 50.5 μmol) were added to DMF (0.8 mL), followed by addition of Et₃N (35 and the resulting mixture stirred at room temperature (about 21° C.) overnight. Solvent was removed by vacuum evaporation, then dried over high vacuum. The resulting product was used directly in the next reaction.

Bn-DOTA·Lu³⁺-PEG 4-NH₂·TFA

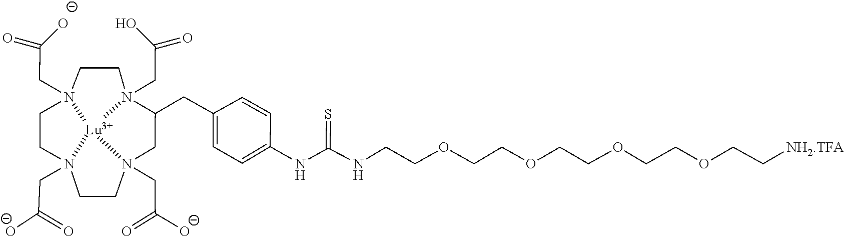

DOTA-Lu³⁺-PEG 4-NHBoc was dissolved in a 4:1 (v/v) solution of DCM/TFA (0.8 mL), and the resulting colorless mixture was stirred at room temperature (about 21° C.) for 40 min. Solvents were then removed by vacuum evaporation, and the residue was purified by HPLC, C-18 reverse phase column, using the gradient 5-40% acetonitrile (containing 0.05% TFA) in water (containing 0.05% TFA). Subsequent lyophilization provided the desired DOTA-Lu³⁺-PEG4-NH₂ TFA salt (21 mg, 53%) as a white foam.

DOTA-Lu³⁺-PEG 4-DFO

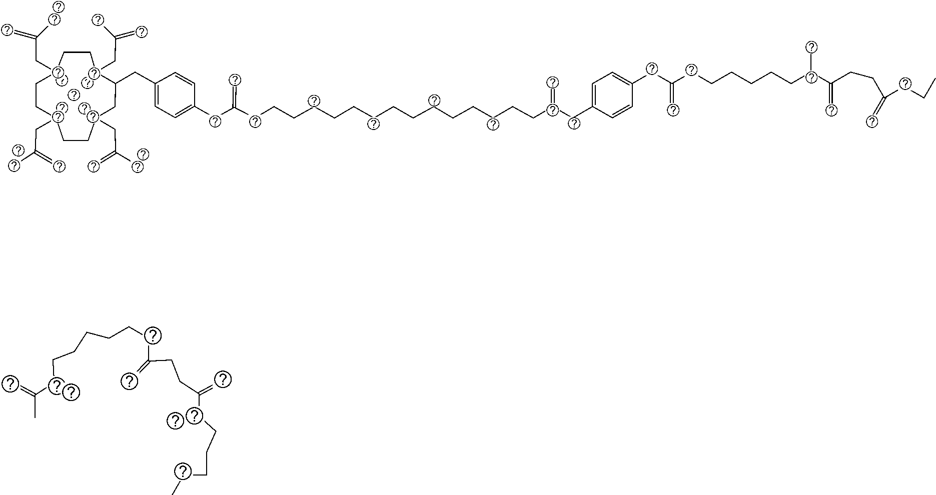

At room temperature (about 21° C.), a solution of Dota-Lu³⁺-PEG (4)-NH₂·TFA salt (21 mg, 21.9 μmol) and DFO-SCN (18 mg, 23.9 μmol) in DMF (0.8 mL) was treated with Et₃N (15 μL), and stirring was at room temperature was maintained for an overnight period. The volatiles were then removed under vacuum, and the residue was then purified by reverse phase HPLC using the gradient 5-50% acetonitrile (containing 0.05% TFA) in water (containing 0.05% TFA). DOTA-Lu³⁺-PEG 4-DFO (37.2 mg, 91%) was isolated as a white foam after lyophilization of the appropriate fractions. 1H NMR, D₂O: 7.23-7.17 (m, 8H), 3.70-2.90 (m, 45H), 2.81-2.30 (m, 19H), 2.16 (d, 1H), 2.02-2.05 (m, 3H), 1.60-1.51 (m, 8H), 1.44-1.39 (m, 4H), 1.22-1.19 (m, 6H). LCMS: Rf: 3.63 Minutes within a 8 minutes' run. MS calculated for C₆₇H₁₀₆LuN₁₅O₂₀S₃ [M+1]⁺=1712.64, [M+1]²⁺=856.32. Found: 856.81. In negative mode: calculated, [M−1]²⁻=855.31. Found: 855.37.

Notably, utilizing different isothiocyanates in a similar reaction with DOTA-Lu³⁺-PEG4-NH₂TFA provides for other compounds and compositions of the present technology. For example, utilizing PCTA-isothiocyanate (illustrated below in Scheme 3) or a salt thereof (e.g., the tris-HCl salt of PCTA-isothiocyanate) instead of DFO-SCN provides DOTA·Lu³⁺-PEG₄-PCTA of the present technology, illustrated in Scheme 3.

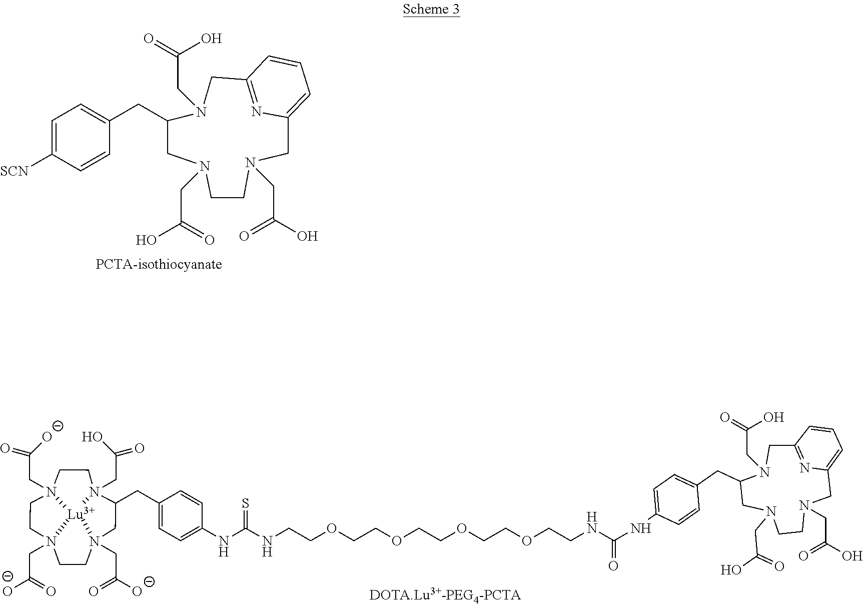

### Example 1B: Synthesis of DOTA·Lu³⁺-PEG 4-DOTA

Scheme 2 provides a synthetic route to provide DOTA·Lu³⁺-PEG 4-DOTA of the present technology. Experimental details of the synthesis are provided thereafter.

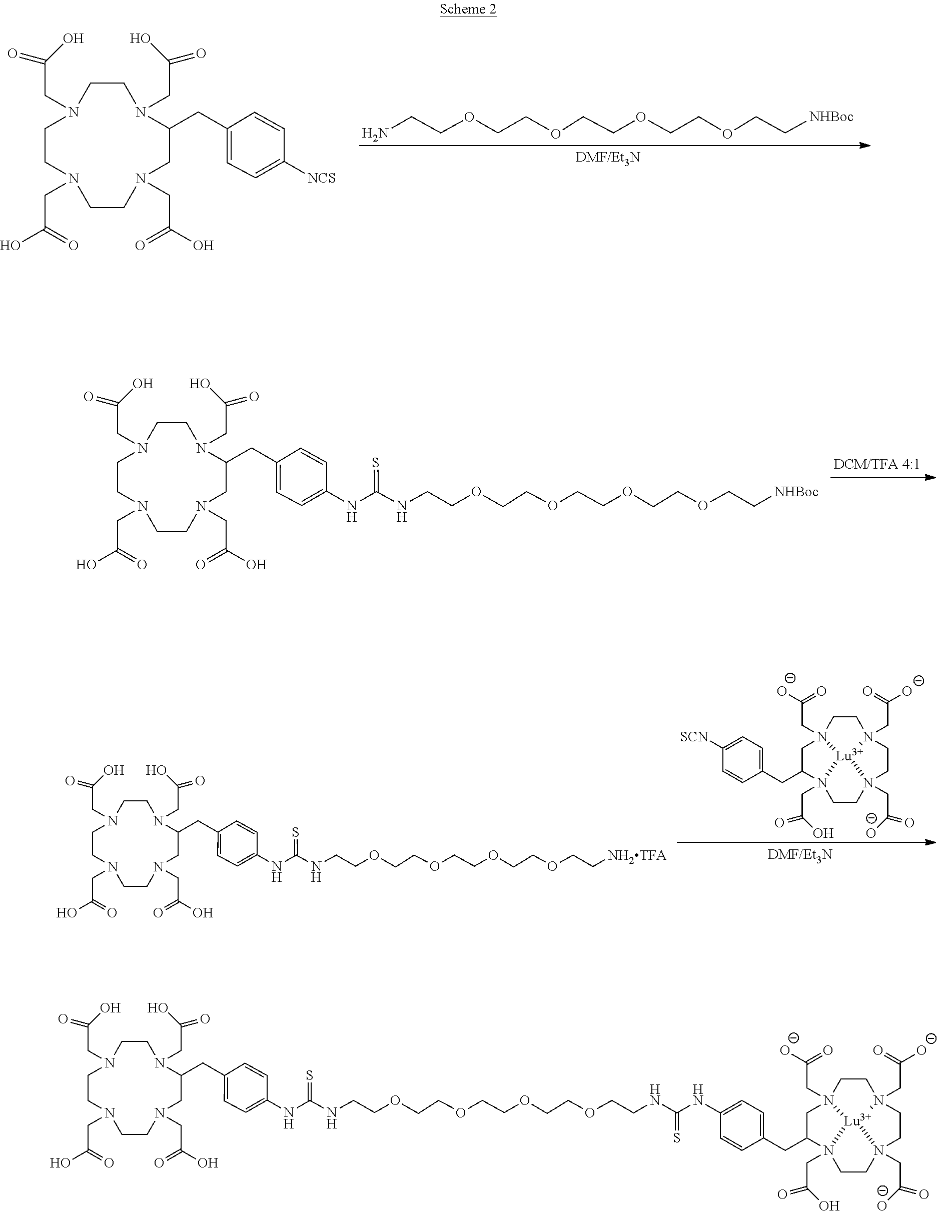

DOTA-PEG 4-NHBoc

At room temperature (about 21° C.), P-SCN-Bn-DOTA (30 mg, 54.4 μmol) and Boc-NH-PEG 4-NH₂ (18 mg, 53.5 μmol) were dissolved in anhydrous DMF (0.7 mL) the resulting solution was treated with Et₃N (36 μL). The mixture was stirred at room temperature overnight. Solvents were then removed by vacuum evaporation, and the residue was dried over high vacuum. This was submitted directly in the next step.

DOTA-PEG 4-NH₂·TFA

DOTA-PEG 4-NHBoc was dissolved in a 4:1 (v/v) DCM/TFA (0.8 mL), and the resulting colorless mixture was stirred at RT for 40 min. The volatiles were then removed by evaporation, and the residue was purified by reverse phase C-18 HPLC using the gradient 5-40% acetonitrile (containing 0.05% TFA) in water (containing 0.05% TFA). DOTA-PEG 4-NH₂·TFA (20 mg, 47%) was obtained after lyophilization of the appropriate fractions.

DOTA·Lu³⁺-PEG 4-DOTA

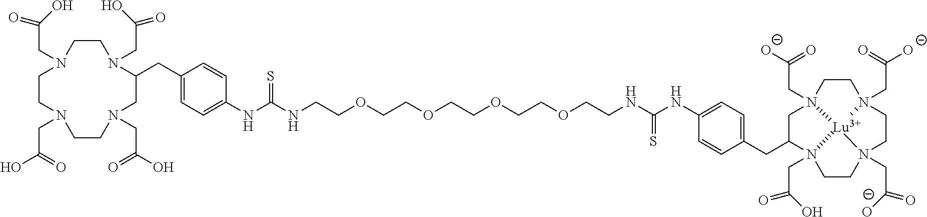

At room temperature (about 21° C.), DOTA-PEG 4-NH₂·TFA salt (20 mg, 25.4 μmol) and DOTA·Lu³⁺-SCN major isomer complex (15.3 mg, 21.1 μmol) were mixed in anhydrous DMF (0.8 mL) and then treated with Et₃N (15 The reaction was stirred room temperature under argon atmosphere overnight. Solvents were then removed by vacuum evaporation, and the residue was purified by reverse phase C-18 HPLC using the gradient 5-50% acetonitrile (containing 0.05% TFA) in water (containing 0.05% TFA). The desired DOTA-PEG 4-DOTA·Lu³⁺ (19.6 mg, 61%) mono-complex was isolated as a white foam upon lyophilization of product-containing fractions. ¹H NMR, D₂O: 7.30-7.15 (m, 8H), 3.75-2.90 (m, 58H), 2.82-2.36 (m, 11H), 2.18-2.14 (m, 1H). The latter multiplet contains some water peaks as well.

LCMS: R_{f}=4.51 minutes on a 8 minutes' HPLC run. MS calculated for C₅₈H₈₇LuN₁₂O₂₀S₂, [M+1]⁺=1511.51, [M+1]²⁺=755.75. Found: 756.25. In negative mode, [M−1]²⁻=754.82. found: 754.75.

### Example 1C: Synthesis of DOTA·Lu³⁺-PEG 4-NODAGA

DOTA·Lu³⁺-PEG 4-NODAGA

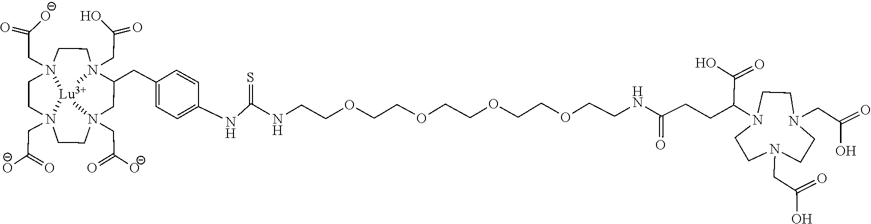

At room temperature (about 21° C.), p-SCN-Bn-DOTA·Lu³⁺ major isomer complex (20 mg, 27.6 μmol) and NH₂-PEG 4-NODAGA (17 mg, 28.6 μmol) were dissolved in anhydrous DMF (0.8 mL) before treatment with Et₃N (20 μL). The resulting mixture was stirred at room temperature for an overnight period. Solvents were then removed by vacuum evaporation, and the colorless residue was purified by reverse phase C-18 HPLC, using the gradient 5-40% acetonitrile (containing 0.05% TFA) in water (containing 0.05% TFA). DOTA·Lu³⁺-PEG 4-NODAGA (15.1 mg, 41%) was obtained as a white foam after lyophilization of the appropriate fractions.

¹H NMR, D₂O: 7.15-7.25 (m, 4H), 3.94-3.91 (m, 1H), 3.89-3.51 (m, 26H), 3.45-2.81 (m, 24H), 2.5-2.35 (m, 12H), 2.20-2.18 (m, 1H), 2.07-2.03 (m, 1H), 1.97-1.94 (m, 1H). Two close isomers are observed in LCMS with the ratios: 18% and 82%. The minor is at 3.02 minutes Rf and the major at 3.08 minutes within the 8 minutes' run.

MS calculated for C₄₉H₇₇LuN₁₀O₁₉S=1316.45. [M+1]⁺=1317.46, [M+1]²⁺=658.73. Found: 659.35.

Alternatively, DOTA-Lu³⁺-PEG4-NH₂TFA may be reacted with the NHS ester of NODAGA (“NODAGA-NHS,” CAS Number 1407166-70-4, illustrated in Scheme 4) and excess base in DMF, and after completion of the reaction (e.g., as indicated by HPLC) utilizing reverse phase C-18 HPLC purification and lyophilization to provide DOTA·Lu³⁺-PEG 4-NODAGA.

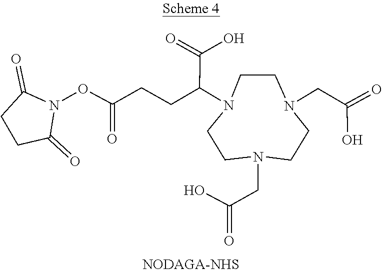 [SMILES: O=C(O)CN1CCN(CC(=O)O)CCN(C(CCC(=O)ON2C(=O)CCC2=O)C(=O)O)CC1]

Notably, utilizing protocols similar to either of the above-described procedures provides for other compounds of the present technology. For example, HOPO-NHS (illustrated in Scheme 5) may be reacted with DOTA-Lu³⁺-PEG4-NH₂TFA and excess base in DMF, and after completion of the reaction (e.g., as indicated by HPLC) utilizing reverse phase C-18 HPLC purification and lyophilization to provide DOTA-Lu³⁺-PEG4-HOPO (as also illustrated in Scheme 5).

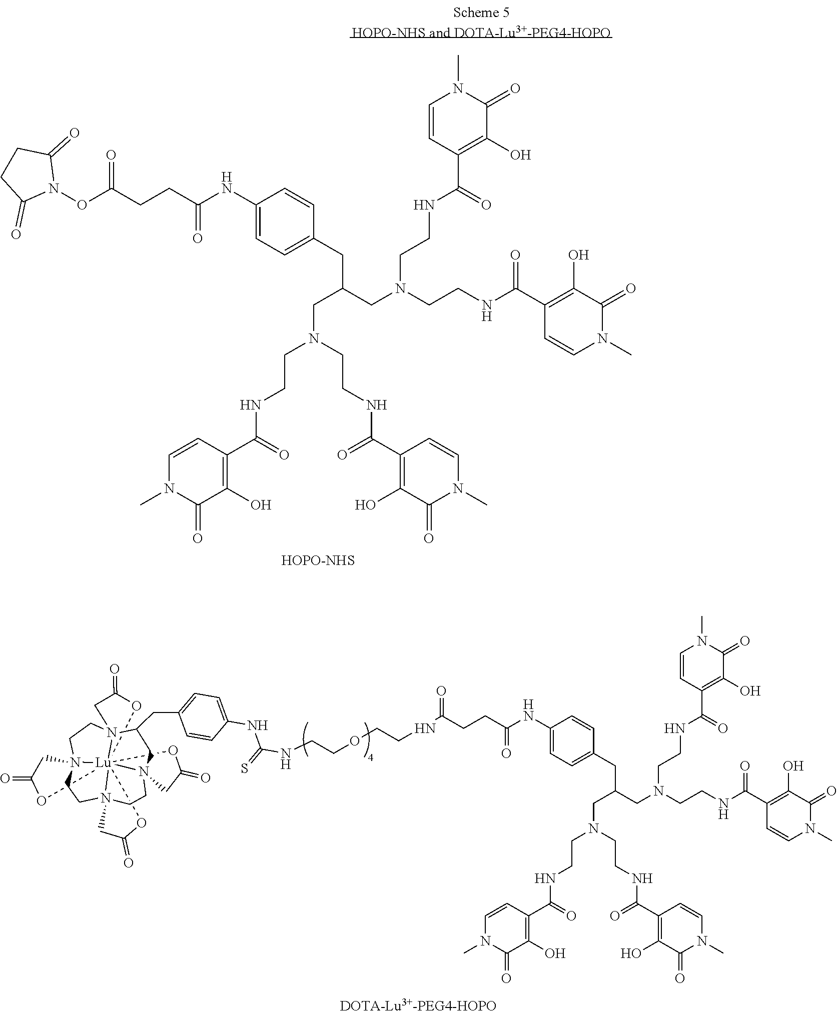

### Example 1D: Synthesis of TCMC-PEG₄-^{nat}LuDOTABn

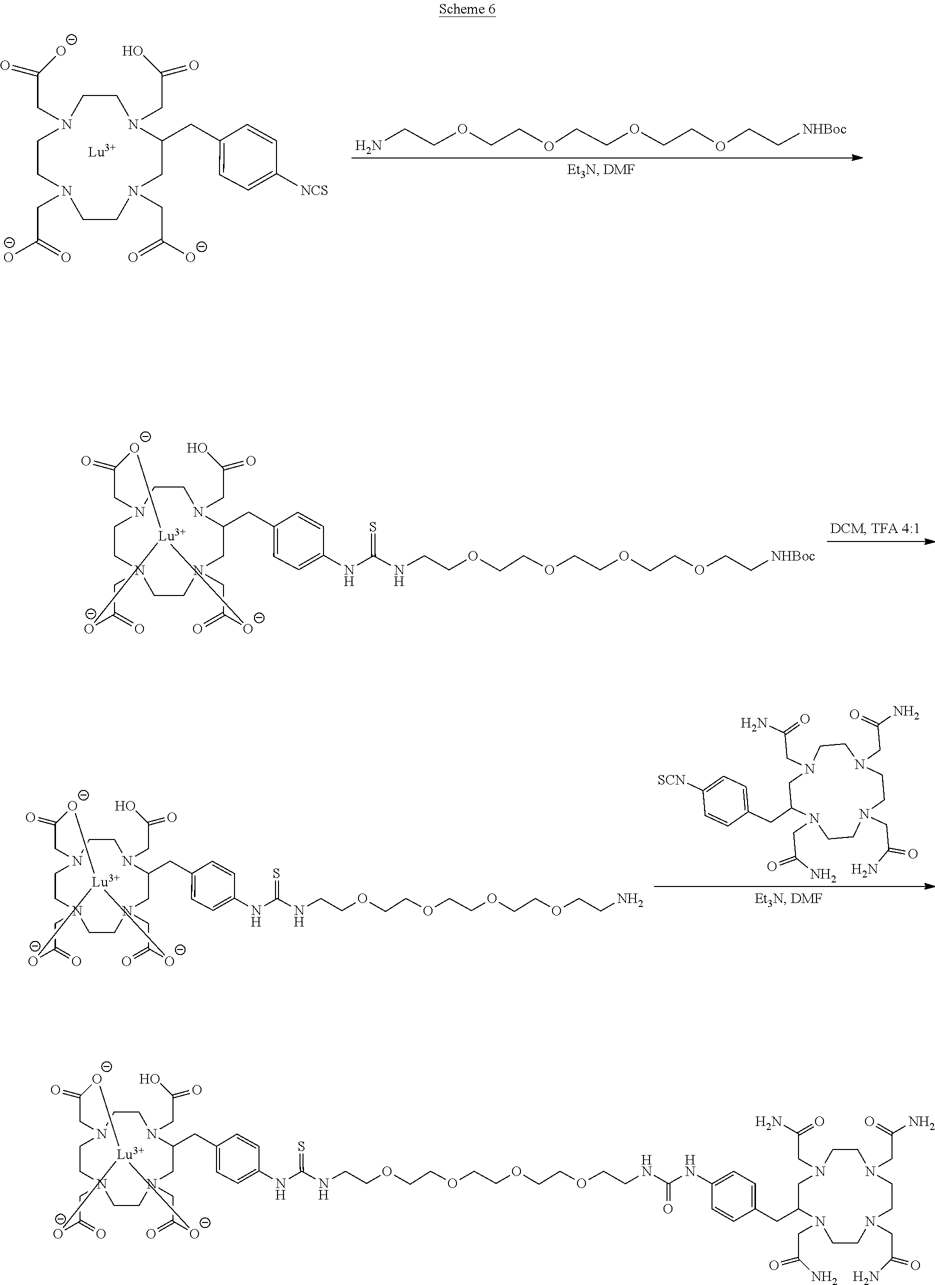

DOTA-Lu³⁺-PEG4-NHBoc:

To DOTA-Lu³⁺-SCN (25.0 mg, 34.6 μmol) and BocNH-PEG4-NH₂ (13.9 mg, 41.3 μmol) in DMF (0.8 mL) was added Et₃N (29 μL). The mixture was stirred at RT for 5 h. Solvents were removed under reduced pressure. The residue was purified by preparative reverse phase C-18 HPLC using a gradient of 20:80 MeCN:H₂O to 40:60 MeCN: H₂O (both containing 0.05% TFA) over 10 min, the product was obtained after lyophilization (14.0 mg, 38%).

DOTA-Lu³⁺-PEG4-NH₂:

DOTA-Lu³⁺-PEG4-NHBoc (14.0 mg, 13.2 μmol) in TFA:DCM (4:1, V:V) was stirred at RT for 40 min, the solvents were then removed under reduced pressure. The residue was dried under high vacuum (2 h) and submitted directly in the next step without further purification.

DOTA-Lu³⁺-PEG4-TCMC

The residue above was dissolved in DMF (0.8 mL), then TCMC-DOTA (10 mg, 18.3 μmol) and Et₃N (40 μL) were added to the mixture. The reaction was stirred at ambient temperature overnight. The volatiles were removed under reduced pressure, and the residue was purified by preparative C-18 reverse phase HPLC using the gradient of 5:95 MeCN:H₂O to 40:60 MeCN: H₂O (both with 0.05% TFA) over 10 min. The product was obtained after lyophilization (16.19 mg, 81%). ¹HNMR (500 MHz, D₂O): δ=7.25-7.18 (m, 8H), 3.82-3.2 (m, 40H), 3.10-2.95 (m, 2H), 2.83-2.38 (m, 28H). MS: calculated: 1507.6 [M+H]⁺. found: 1507.5.

### Example 1E: Radiosynthesis of Compounds of Present Technology

Radiochemistry was performed in appropriately shielded chemical fume hoods equipped with electronic flow monitoring and sliding leaded glass windows. A CRC-55tR dose calibrator was used to measure radioactivity using manufacturer recommended calibration settings (Capintec Inc., Florham Park, NJ). Buffers and water used for radiochemical synthesis were treated with 5% w/v Chelex ion exchange resin (BT Chelex 100 Resin, Bio-Rad Inc., Hercules, CA) to remove adventitious heavy metals. Plasticware (pipet tips and microcentrifuge tubes) were tracemetal grade/RNA grade. RadioHPLC was performed on a Shimadzu Prominence HPLC system comprised of an LC-20AB dual pump module, DGU-20A3R degasser, SIL-20ACHT autosampler, SPD-20A UV-Vis detector and a Bioscan Flow-Count B-FC-1000 with PMT/NaI radioactivity detector in-line. Separations were run on an analytical 4.6×250 mm Gemini-NX C18 or Fusion RP C18 HPLC column (Phenomenex, Inc. Torrance, CA). Unless otherwise noted, HPLC conditions were: solvent A—10 mM pH 5 NH₄OAc, B—CH₃CN, 1.0 mL/min flow rate, λ=254 nm, injection volume 10-50 μL, gradient: 0% B to 40% B over 10 min. Samples of free radiometals, reaction mixtures and purified products were diluted 1:5 in 5 mM DTPA prior to analysis.

Radiosynthesis of [²⁰³Pb]TCMC-PEG4-LuDOTA

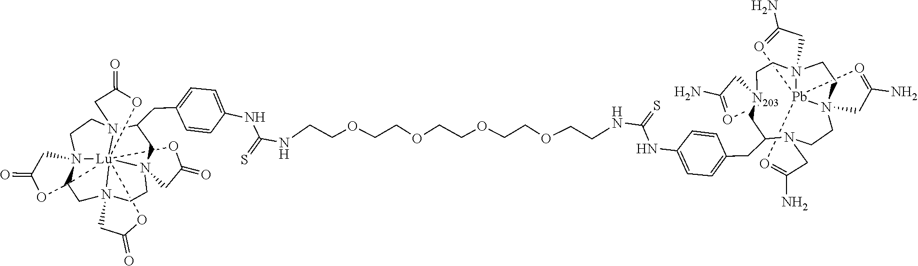

²⁰³PbCl₂ (39.2 MBq/1.06 mCi) in 154, of 0.5M HCl (Lantheus Medical Imaging, Billerica MA) was transferred to a metal-free 1.5 mL microcentrifuge tube and diluted with 2004, of chelexed aqueous 0.5M NH₄OAc (pH 5.3) and mixed gently. To this was added 104, of 1 mM TCMC-PEG4-LuDOTA (10 nmol) and mixed gently and placed in a heat block set to 40° C. After 30 minutes, the reaction was cooled briefly, then the entirety was gravity loaded on a 30 mg Strata-X SPE cartridge (Phenomenex, Torrance CA), which had been equilibrated with 1 mL of ethanol and 1 mL of water. Water (100 μL) was used to rinse the reaction tube and passed through the cartridge. The column was washed slowly dropwise with 200 μL of water, the column purged gently with nitrogen gas, then the product was slowly eluted dropwise with 2004, of ethanol into a clean 2 mL microfuge tube and diluted to 2.0 mL with normal saline (Hospira, Lake Forest, IL) and sterile filtered to obtain [²⁰³Pb]TCMC-PEG4-LuDOTABn (36.1 MBq (975 μCi), 92% yield, A_{M}=3.9MBq/nmol (106 μCi/nmol)). RadioHPLC confirmed that no free radiometal remained (98.1% radiochemical purity; major isomer t_{R}=10.8 min).

Radiosynthesis of [⁸⁹Zr]DFO-PEG₄-LuDOTA

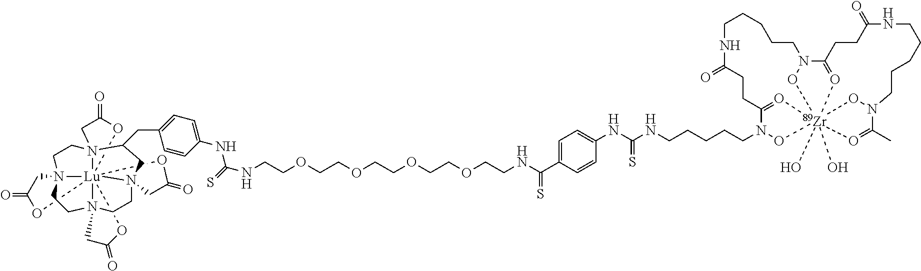

[⁸⁹Zr]ZrOxalate₂ (67.7 MBq/1.83 mCi) in 50 μL of 1.0M oxalic acid (Cyclotron Core Facility MSKCC) was transferred to a metal-free 1.5 mL microcentrifuge tube and neutralized with an equimolar amount of metal-free 1.0M Na₂CO₃ ˜45 μL, then diluted with 4004, of metal-free 0.5M HEPES buffer (pH 7.5) and mixed. To this was added DFO-PEG4-LuDOTA (9.2 nmol, 9.2 μL of 1.0 mM solution in water), mixed and placed in a heat block at 40° C. After 60 minutes, the entirety was gravity loaded on a 30 mg Strata-X SPE cartridge (Phenomenex, Torrance CA), which had been equilibrated with 1 mL of ethanol and 1 mL of water. Water (100 μL) was used to rinse the reaction tube and passed through the cartridge. The SPE cartridge was washed with 200 μL of water, gently blown dry with nitrogen gas, then the product was slowly eluted dropwise with 200 μL, of ethanol into a clean 2 mL microfuge tube. The eluent was diluted into 2 mL with normal saline (Hospira, Lake Forest, IL) and sterile filtered to obtain 44 MBq (1.2mCi; 66% yield, A_{M}=7.4MBq(0.2mCi)/nmol) of [⁸⁹Zr]DFO-PEG₄-LuDOTA. This stock was used to prepare the doses for PET imaging and biodistribution (3.7MBq/100 μCi; 0.5 nmol). RadioHPLC (solvent A: 0.1% TFA, B: CH₃CN) of crude and purified material confirmed that no detectable free radiometal remained (major isomer t_{R}=10.7 min, 99+% conversion).

Radiosynthesis of [¹⁷⁷Lu]DOTABn-PEG₄-LuDOTA

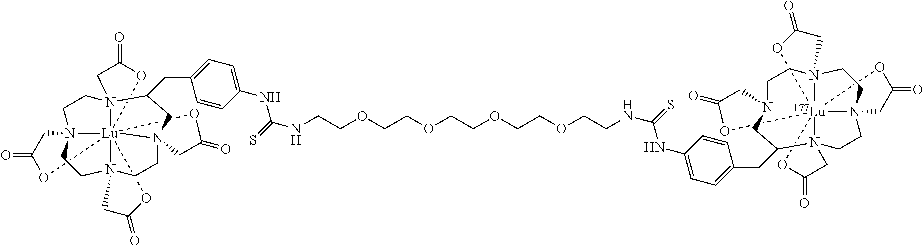

[¹⁷⁷Lu]LuCl₃ (38 MBq/1.03 mCi) in 19 μL of 0.05M HCl (NIDC/MURR; Missouri University Research Reactor, Columbia, MO) was transferred to a metal-free 1.5 mL microcentrifuge tube and diluted with 100 μL, of metal-free 0.5M NH₄OAc (pH 5.3) and mixed gently. To this was added DOTABn-PEG₄-LuDOTABn (5 nmol, 5 μL of 1 mM solution in water), and mixed gently and placed in a heat block at 80° C. for 60 minutes. After cooling for 5 minutes, the entirety was gravity loaded on a 30 mg Strata-X SPE cartridge (Phenomenex, Torrance CA), which had been equilibrated with 1 mL of ethanol and 1 mL of water. Water (100 μL) was used to rinse the reaction tube and passed through the cartridge. The column was washed slowly dropwise with 200 μL of water, gently blown dry with nitrogen gas. The product was slowly eluted dropwise with 200 μL, of ethanol into a clean 2 mL microfuge tube and diluted to 2.0 mL with normal saline (Hospira, Lake Forest, IL) and sterile filtered to obtain [¹⁷⁷Lu]DOTABn-PEG₄-^{nat}LuDOTA (33.7 MBq (0.91mCi), 88% yield, A_{M}=7.4MBq/nmol (0.2mCi/nmol)). RadioHPLC of crude and purified material confirmed that no free radiometal remained (99+% radiochemical purity; major isomer t_{R}=9.3 min).

Radiosynthesis of [⁸⁶Y]DOTABn-PEG₄-LuDOTA

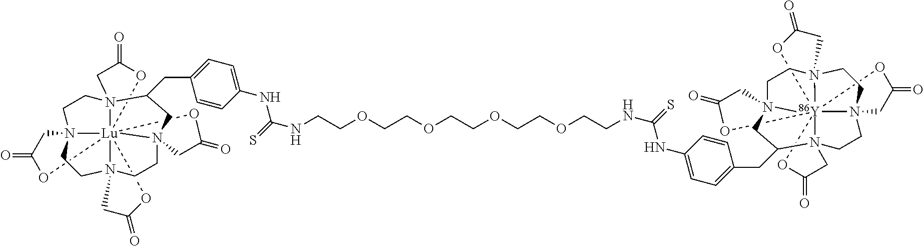

[⁸⁶Y]YCl₃ (4.7 MBq/126 μCi) in 5 μL of 0.04M HCl (MDACC CRF; Cyclotron Radiochemistry Facility MD Anderson Cancer Center, Houston, TX) was transferred to a metal-free 0.5 mL microcentrifuge tube and diluted with 50 μL of metal-free 0.5M NH₄OAc (pH 5.3) and mixed gently. To this was added DOTABn-PEG4-LuDOTABn (2 nmol, 2 μL of 1 mM solution in water), and mixed gently and placed in a heat block at 80° C. for 60 minutes. After cooling for 5 minutes, the entirety was gravity loaded on a 30 mg Strata-X SPE cartridge (Phenomenex, Torrance CA), which had been equilibrated with 1 mL of ethanol and 1 mL of water. Water (100 μL) was used to rinse the reaction tube and passed through the cartridge. The column was washed slowly dropwise with 200 μL of water, gently blown dry with nitrogen gas. The product was slowly eluted dropwise with 200 μL of ethanol into a clean 2 mL microfuge tube and diluted to 2.0 mL with normal saline (Hospira, Lake Forest, IL) and sterile filtered to obtain [⁸⁶Y]DOTABn-PEG₄-^{nat}LuDOTA (1.38 MBq (37.2 μCi), 29% yield, A_{M}=2.3MBq/nmol (63 μCi/nmol)). RadioHPLC confirmed that no free radiometal remained (99+% radiochemical purity; major isomer t_{R}=9.15 min).

Radiosynthesis of [⁶⁸Ga]NODAGA-PEG₄-LuDOTA

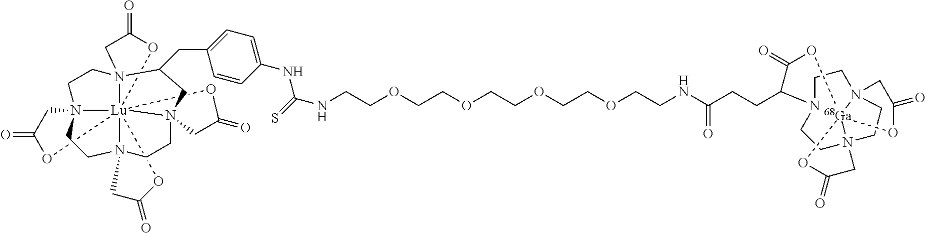

[⁶⁸Ga]GaCl₃ (175 MBq/4.7 mCi) in 1 mL 0.1M HCl was eluted from a GalliaPharm ⁶⁸Ge/⁶⁸Ga generator (Eckert & Ziegler Radiopharma GmbH, Berlin, Germany) was transferred to a metal-free 2 mL microcentrifuge tube and diluted with 500 μL of chelexed aqueous 0.5M NH₄OAc (pH 5.3) and mixed gently. To this was added NODAGA-PEG₄-LuDOTA (2 nmol in 20 μL water) and mixed gently. The tube was placed in a heat block at 80° C. for 15 minutes. After cooling for 5 minutes, the entirety was gravity loaded on a 30 mg Strata-X SPE cartridge (Phenomenex, Torrance CA), which had been equilibrated with 1 mL of ethanol and 1 mL of water. Water (100 μL) was used to rinse the reaction tube and passed through the cartridge. The column was washed with 200 μL of water, blown dry with nitrogen gas, then the product was slowly eluted dropwise with 200 μL of ethanol into a clean 1.5 mL microfuge tube. The volume of eluent was reduced under dry nitrogen gas flow to approximately 50 μL, diluted into 2 mL of normal saline (Hospira, Lake Forest, IL) and sterile filtered to obtain 141 MBq (3.8mCi; 81% yield, A_{M}=65MBq/nmol (1.8mCi/nmol)) of [⁶⁸Ga]NODAGA-PEG4-LuDOTA. This stock was used to prepare the doses for PET imaging (9.6MBq/260 μCi; 0.15 nmol) and was diluted further in sterile saline for biodistribution doses (6.5MBq/175 μCi; 0.1 nmol). RadioHPLC of crude and purified material confirmed that no free radiometal remained (major isomer t_{R}=8.1 min, 99+% conversion).

Radiosynthesis of [⁶⁴Cu]NODAGA-PEG₄-LuDOTA

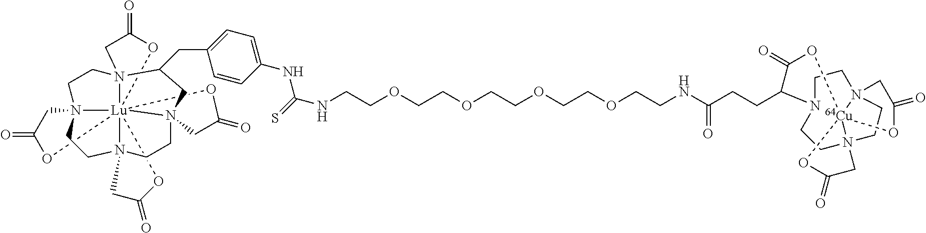

[⁶⁴Cu]CuCl₂ (38.1 MBq/1.03 mCi) in 4 μL (Washington University St. Louis) was transferred to a metal-free 1.5 mL microcentrifuge tube and diluted with 304, of chelexed aqueous 0.5M NH₄OAc (pH 5.3) and mixed gently. To this was added NODAGA-PEG4-LuDOTA (3 nmol) in 30 μL buffer, and mixed gently. After 5 minutes, the entirety was gravity loaded on a 30 mg Strata-X SPE cartridge (Phenomenex, Torrance CA), which had been equilibrated with 1 mL of ethanol and 1 mL of water. Water (100 μL) was used to rinse the reaction tube and passed through the cartridge. The column was washed slowly dropwise with 200 μL of water, gently blown dry with nitrogen gas, then the product was slowly eluted dropwise with 200 μL of ethanol into a clean 1.5 mL microfuge tube. The volume of eluent was reduced under dry nitrogen gas flow to approximately 50 μL, diluted into normal saline (Hospira, Lake Forest, IL) and sterile filtered to obtain 26.1 MBq (0.71mCi; 68% yield) of [⁶⁴Cu]NODAGA-PEG4-LuDOTA. This stock was used to prepare the doses for PET imaging (11MBq/300 μCi; 1 nmol) and was diluted further in sterile saline for biodistribution doses (1.9MBq/51 μCi; 0.15 nmol). RadioHPLC of crude and purified material confirmed that no free radiometal remained (99+% radiochemical purity; A_{M}=12.7MBq/nmol).

### Example 2: Radiotracer Targeting in In Vitro and Xenograft Models

A biotinylated DOTA-based hapten probe was utilized for flow cytometry characterization (FACS) of C825-transduced (CAR-T) cells in vitro to verify successful C825-transduction and assay for functional C825-hapten binding.

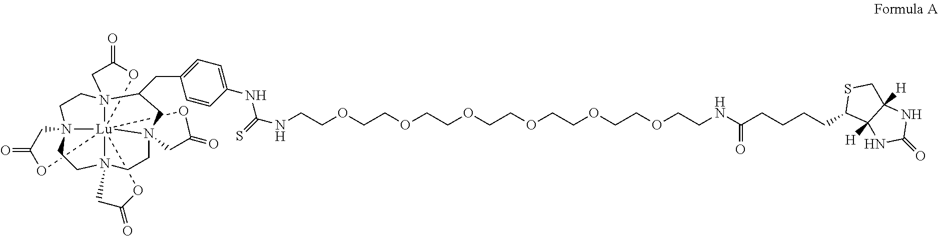

The probe may also be used for histological assay (e.g., Axworthy D B Proc Natl Acad Sci 2000 Feb. 15; 97(4):1802-7). Lu may be substituted with Gd.

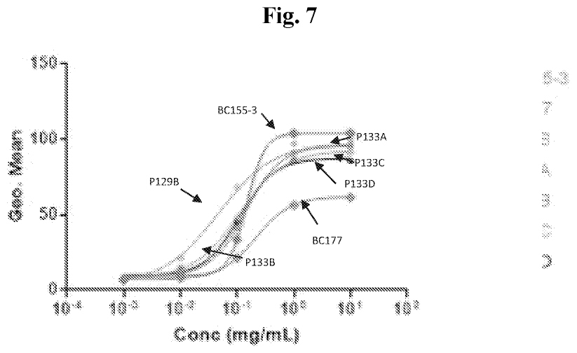

Functional C825-hapten binding was assessed via FACS using several anti-GPA33/C825 bispecific antibodies. GPA33-expressing Colo205 cells were harvested and viability was assessed. 1 million live cells per FACS tube were spun down in ice-cold phosphate buffered saline without Ca and Mg (PBS) (500 g, 5 min), 1 tube per sample. Supernatant was decanted and primary antibody (1 μg in 100 μl of PBS, per sample for 0.01 mg/mL or 10-2 mg/mL) was added, vortexed and incubated at 4° C. for 20 minutes. Tubes were filled with PBS and spun down once to wash out primary antibody (500 g, 5 min). Supernatant was decanted and biotin-DOTA(Lu) probe (0.1 μg in 100 μg of PBS, per sample) was added, vortexed and incubated at 4° C. for 20 minutes. Tubes were filled with PBS and spun down once to wash out biotin-DOTA(Lu) probe (500 g, 5 min). The PBS wash step was repeated. Supernatant was decanted and streptavidin-phycoerythrin (streptavidin-PE; BD catalog 554061; 1:500 dilution in 100 μl of PBS, per sample) was added, vortexed and incubated at 4° C. for 20 minutes. Tubes were filled with PBS and spun down once to wash out streptavidin-PE (500 g, 5 min). Supernatant was decanted and 200-500 μl of PBS was added. Samples were vortexed and evaluated on a flow cytometer. PBS and single-color controls may be prepared for comparison. Results were analyzed with FlowJo software (FlowJo LLC, 220 Ashland, Oregon). Results are shown in FIG. 7.

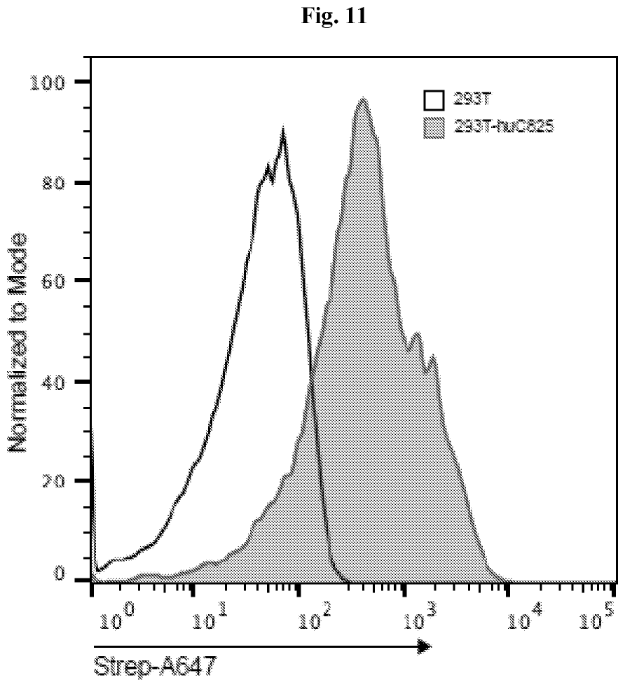

A mean Kd (equilibrium dissociation constant) of 240±122 pM, a mean Bmax (sites/cell) of 17 000±4400, and a mean R2 of 0.983±0.006 was determined in three independent saturation binding assays. In addition, the binding capability of huC825 for hapten by flow cytometry was assayed using DOTA-PEG6 Biotin in combination with streptavidin-fluorophore (FIG. 11).

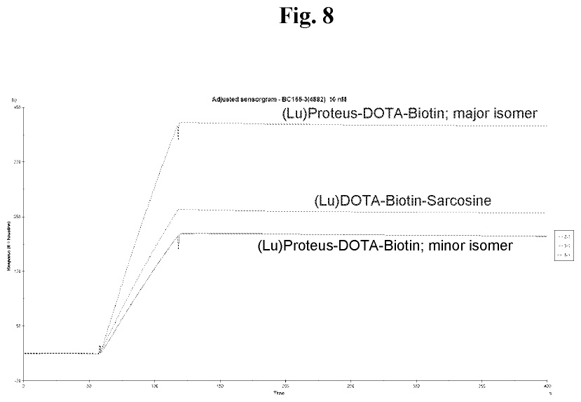

Biacore method: SA chip (Streptavidin) (GE Healthcare) was coated with either of the (Lu)Proteus-DOTA-Biotin (Formula A) and (Lu)DOTA-Biotin-Sarcosine and then flowed over with BC155 BsAb (a GPA33×C825 IgG-scFv). The curve shows the 10 nM concentration. KDs were generated from a series containing 6 concentrations. See Table below and FIG. 8.

| GPA33-C825 IgG-L-scFv (BC155-3) | KD |
|---|---|
| (Lu)DOTA-Biotin-Sarcosine | 5.82E−11 |
| (Lu)Proteus-DOTA-Biotin | 1.93E−11 |

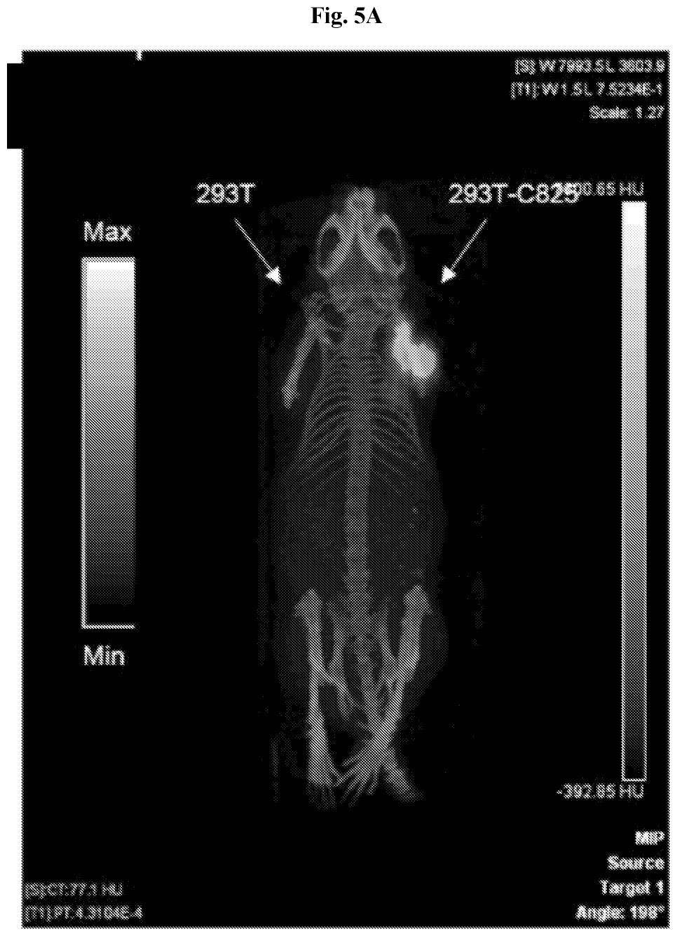

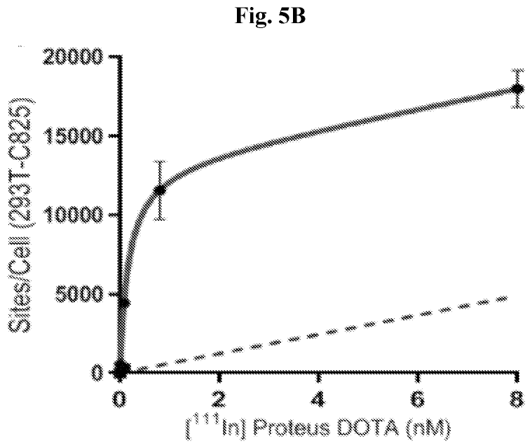

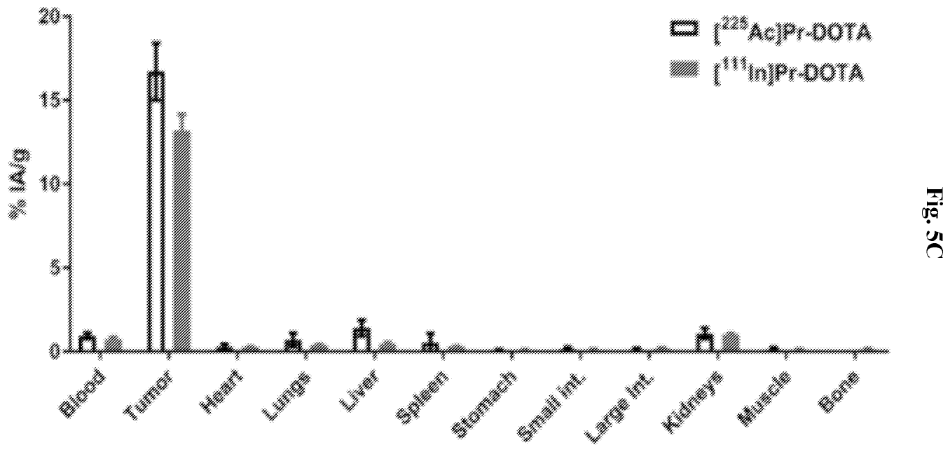

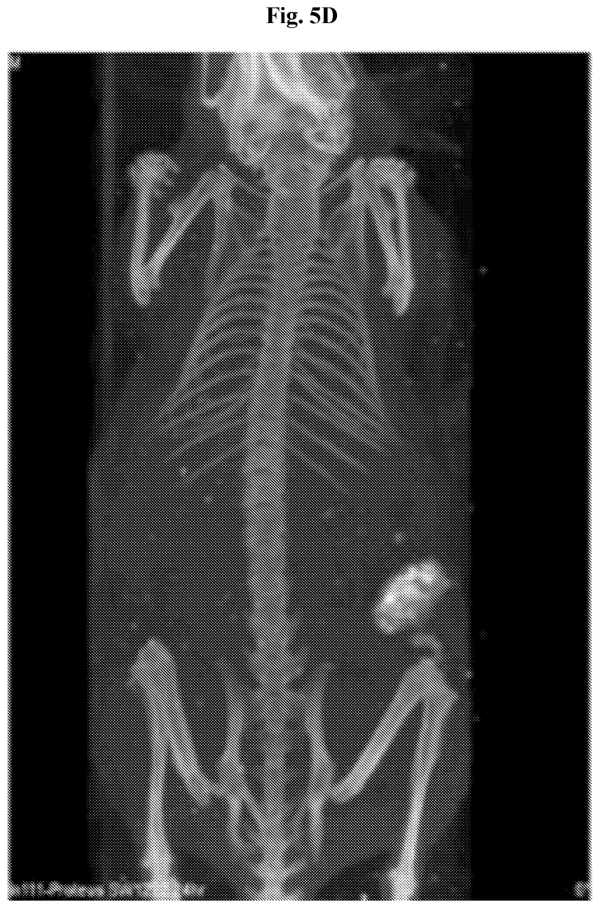

FIG. 5A shows a maximum intensity projection (MIP) PET/CT of a mouse bearing bilateral 293T only/293T-C825 xenografts in the upper shoulders approximately 18 h post-injection of [⁸⁶Y]DOTA-benzene. The 293T-C825 tumor uptake determined by ex vivo biodistribution immediately following imaging was ˜14% ID/g. The detectable signal was observed in the shoulder containing 293T tumor cells only. This was consistent with the results of the in vitro saturation binding assay for [¹¹¹In]Pr-DOTA. See FIG. 5B showing that 293T-C825 cells had a significantly greater number of sites for [¹¹¹In]Pr-DOTA capture compared to the 293T negative control.

FIG. 5C shows a comparison of biodistribution of tracer pretargeted [²²⁵Ac]Pr-DOTA or [¹¹¹In]Pr-DOTA in groups of SW1222 human colorectal cancer tumor-bearing athymic nude mice at 24 h p.i. Following i.v. injections of huA33-C825 bispecific antibody (0.25 mg, 1.19 nmol), clearing agent, and radiolabeled DOTA haptens, the animals were euthanized 24 h later for organ collection and assayed for radioactivity. Data is presented as mean±SEM. FIG. 5D shows an MIP SPECT/CT image approximately 24 h p.i. of pretargeted [¹¹¹In]Pr-DOTA in a SW1222 tumor-bearing athymic nude mouse. The SW1222-xenograft can be clearly delineated in the flank.

### Example 3: In Vivo Tracking of CAR T Cells Transduced with C825 scFv Construct in Xenograft Models

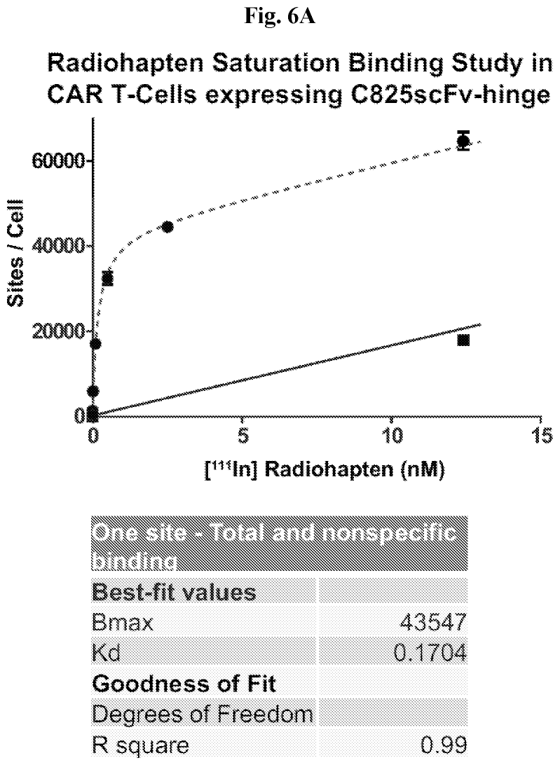

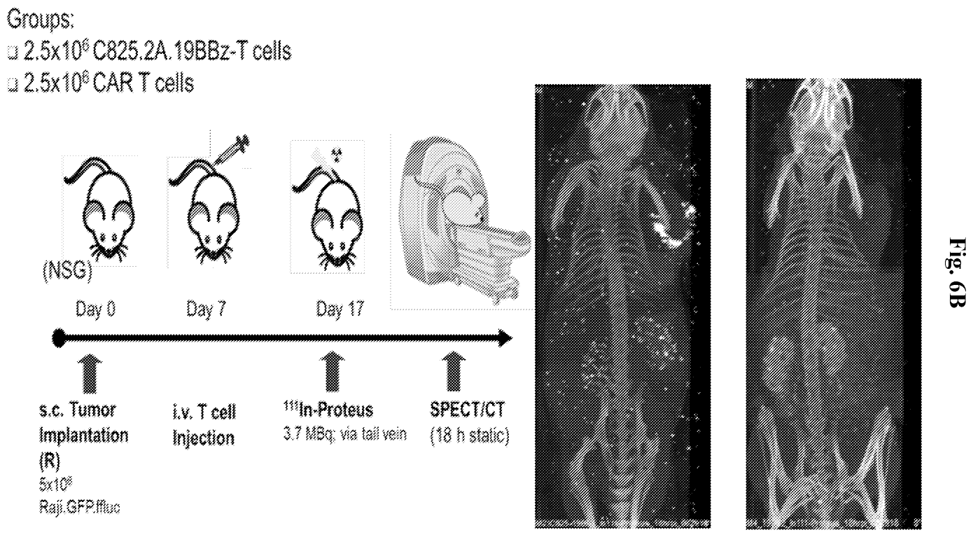

CD-19 CAR-T cells were transduced with a specialized ultra-high affinity membrane expressing hapten capture antibody C825. These cells were purified and tested for surface vector expression using [¹¹¹In]Pr-DOTA radiohapten system prior to in vivo use, by saturation binding assay as shown in FIG. 6A. CD19-expressing Raji lymphoma, a human B-cell (Burkitt's type) lymphoma, were used as a lymphomatous tumor in immunologically deficient mice for CD19 targeting. FIG. 6B shows a NSG mouse with a s.c. Raji GFP-fluc tumor in the right shoulder. Ten days after i.v. CAR-T cell injection (2.5×10⁶), the mouse was injected with [¹¹¹In]Pr-DOTA radiohapten for in vivo tracking of CAR T cells (either: CD19 CAR+C825, or control CD19 CAR only). As shown in FIG. 6C, animals treated with CD19 CAR T cells expressing C825 scFv showed effective tumor targeting in xenografts bearing Raji tumors. CAR-T cells efficiently captured radiohapten chelates with optimized pharmacology via renal clearance (data not shown).

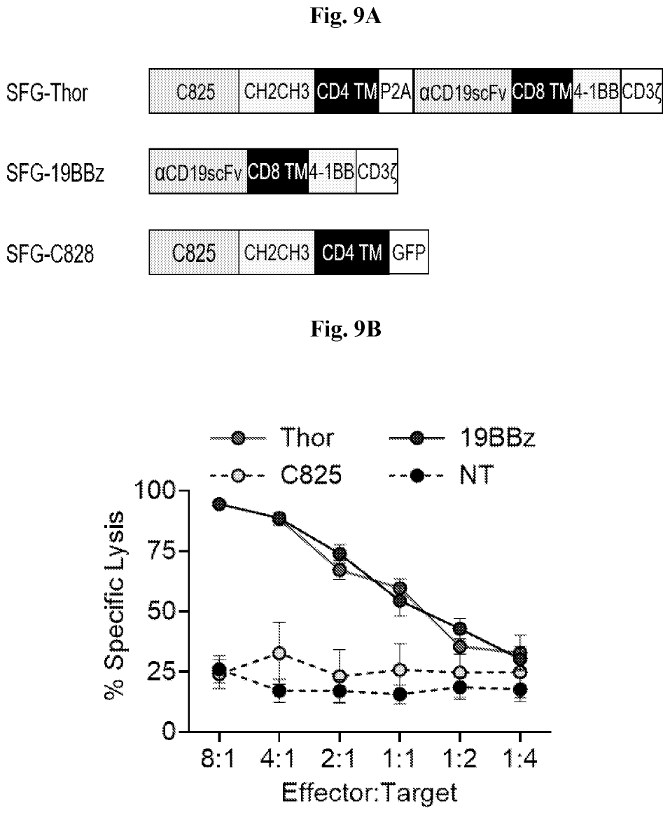

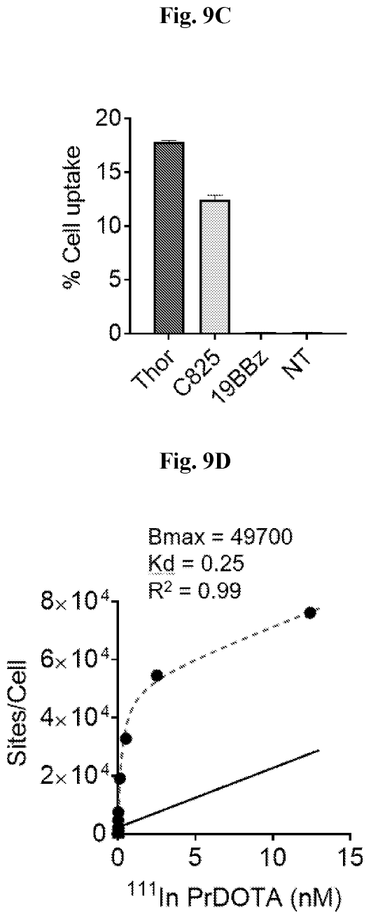

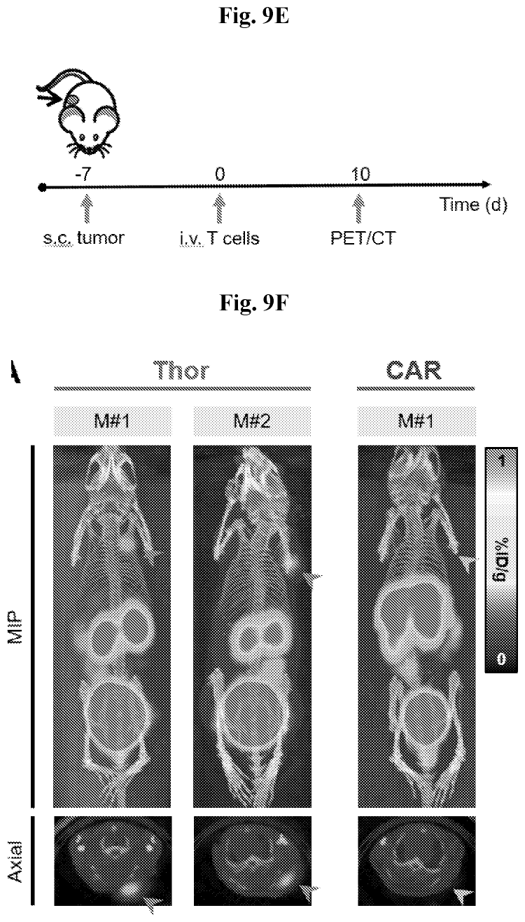

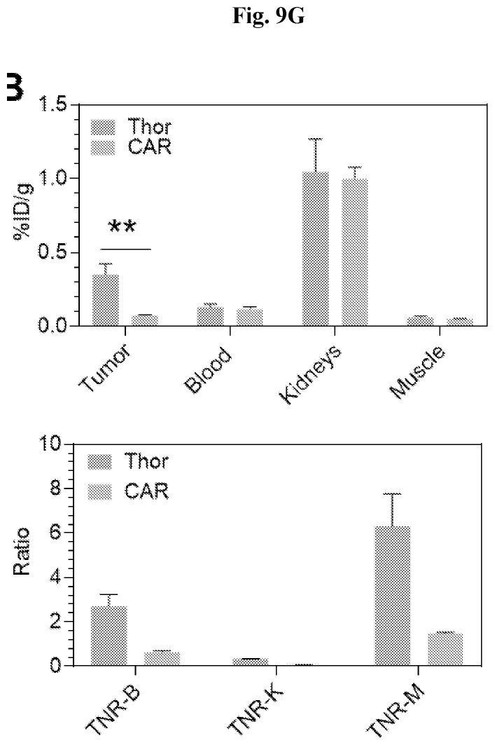

FIG. 9A shows schematic structures of retroviral vectors SFG-Thor, SFG-19BBz (CAR) and SFG-C825. FIG. 9B shows that there is no difference between SFG-Thor T cells and SFG-19BBz (CAR) T cells with respect to killing CD19(+) Raji tumor cells as measured by in vitro 4 h cytotoxicity assays. These results demonstrate that transducing CD19-specific CAR T cells with humanized C825 scFv did not negatively impact their ability to target and lyse CD19(+) tumor target cells. FIG. 9C shows in vitro binding of [¹¹¹In]InPr at 1 h. This representative data set demonstrates the specific binding of the radiolabeled DOTA probe to C825-expressing T cells, whereas no significant uptake was observed in SFG-19BBz (CAR) and NT T cells. (All experiments were performed in triplicate at 37° C.). Data are mean±SD. FIG. 9D shows in vitro binding kinetics of [¹¹¹In]InPr to SFG-Thor T cells (n=3 independent assays; representative example shown). FIG. 9E shows an exemplary scheme of in vivo study for assessing T cell targeting to tumor cells. ⁶⁸Ga-NODAGA-Pr (100 mCu, 700 pmol) was used as the radiotracer and administered 10 days after T cell administration (1×10⁶T cells) in NSG mice bearing CD19(+) Raji xenografts. FIG. 9F shows exemplary Maximum intensity projection (MIP) images at 1 h post-injection (p.i.) of ⁶⁸Ga-NODAGA-Pr depicting homing and accumulation of SFG-Thor T cells at the tumor (right shoulder, red arrow). No uptake above background at the tumor site is noted following SFG-19BBz (CAR) T cell administration (blue arrow). FIG. 9G shows mean uptake in tumors and tumor-to-normal-tissue-ratios (TNR) (SFG-Thor: n=4; SFG-19BBz (CAR): n=2) using image-based biodistribution. **, P<0.01.

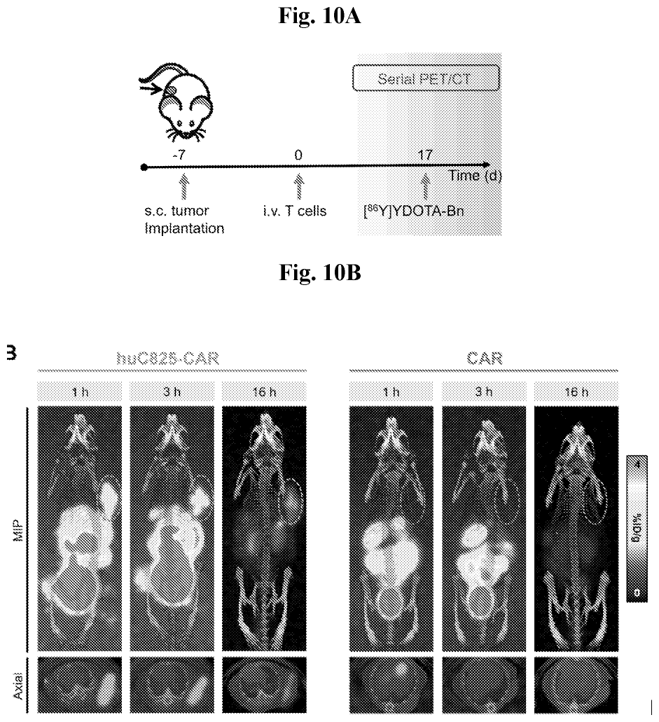

FIG. 10A shows an exemplary scheme for tracking engineered T cells in vivo in a s.c. Raji-tumor mouse model (3×10⁶ cells) with established treatment failure. Seven days post tumor inoculation, mice were injected i.v. with either 3×10⁶ huC825-19BBz or 3×10⁶ 19BBz T cells. On day 17 post T cell administration, mice demonstrating persistent growing tumor burden indicating treatment failure were i.v. injected with ⁸⁶Y-DOTA-Bn (3.7 MBq; 40 pmol) to assess persistence and localization of the transplanted T cells. FIG. 10B shows Maximum intensity projection (MIP) and axial PET/CT images at 1, 3 and 16 h p.i. depict accumulation of huC825-19BBz-CAR T cells at the tumor (orange circle). Highest intratumoral T cell uptake was seen at 3 h pi of 4.9% ID/g (vs 0.8% ID/g in control). No uptake above background at the tumor is noted in control mice (19BBz CAR; green circle). Rapid, predominant renal tracer clearance was noted.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A kit comprising
   (a) a first expression vector comprising a first recombinant nucleic acid sequence that encodes a first fusion protein comprising a DOTA binding fragment fused to a first transmembrane domain, and a reporter gene, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively; and
   (b) a biotinylated DOTA-based hapten; and
   (c) a DOTA-bearing bischelate.
B. The kit of Paragraph A, wherein the first fusion protein further comprises a receptor that binds to a target antigen.
C. The kit of Paragraph A, further comprising a second expression vector comprising a second recombinant nucleic acid sequence that encodes a second fusion protein comprising a receptor that binds to a target antigen.
D. A kit comprising
   (a) a first recombinant nucleic acid sequence encoding a first fusion protein comprising a DOTA binding fragment fused to a glycosylphosphatidylinositol (GPI)-anchored polypeptide, and a receptor that binds to a target antigen, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively; and
   (b) a biotinylated DOTA-based hapten; and
   (c) a DOTA-bearing bischelate.
E. The kit of any one of Paragraphs A-D, wherein the receptor is a T cell receptor, a native cell receptor, a non-native cell receptor, or a chimeric antigen receptor.
F. The kit of Paragraph E, wherein the chimeric antigen receptor comprises (i) an extracellular antigen binding domain; (ii) a second transmembrane domain; and (iii) an intracellular domain.
G. The kit of Paragraph F, wherein the extracellular antigen binding domain binds to the target antigen.
H. The kit of any one of Paragraphs B-G, wherein the target antigen is a tumor antigen.
I. The kit of Paragraph H, wherein the tumor antigen is selected from the group consisting of 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-7A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1.
J. The kit of any one of Paragraphs F-I, wherein the extracellular antigen binding domain comprises a single chain variable fragment (scFv).
K. The kit of any one of Paragraphs F-J, wherein the extracellular antigen binding domain comprises a human scFv.
L. The kit of any one of Paragraphs F-K, wherein the extracellular antigen binding domain comprises a CD19 scFv of SEQ ID NO: 27 or SEQ ID NO: 28.
M. The kit of any one of Paragraphs F-L, wherein the extracellular antigen binding domain comprises a CD19 scFv having at least 80%, 85%, 90%, 95%, 96%, 97%, 98%, or 99% sequence identity to SEQ ID NO: 27 or SEQ ID NO: 28.
N. The kit of any one of Paragraphs F-M, wherein the extracellular antigen binding domain comprises a signal peptide that is covalently joined to the N-terminus of the extracellular antigen binding domain.
O. The kit of any one of Paragraphs F-N, wherein the second transmembrane domain comprises an amino acid sequence that is at least 90% identical to an amino acid sequence of a transmembrane region of CD8, CD28, CD3ζ, CD4, or 4-1BB ligand receptor.
P. The kit of any one of Paragraphs F-O, wherein the intracellular domain comprises one or more costimulatory domains.
Q. The kit of any one of Paragraphs F-P, wherein the one or more costimulatory domains are selected from a CD28 costimulatory domain, a CD3-chain, a 4-1BBL costimulatory domain, or any combination thereof.
R. The kit of any one of Paragraphs A-C and E-Q, wherein the DOTA binding fragment is located at the N-terminus of the first transmembrane domain or the glycosylphosphatidylinositol (GPI)-anchored polypeptide.
S. The kit of any one of Paragraphs A-R, wherein the first and/or second fusion protein further comprises an endoplasmic reticulum signal sequence.
T. The kit of any one of Paragraphs A-S, wherein the sequence of an intra-peptide linker between the V_{H} and the V_{L} in the DOTA binding fragment is any one of SEQ ID NOs: 9-11.
U. The kit of any one of Paragraphs A-T, wherein the V_{H} is located at the N-terminus or the C-terminus of the V_{L}.
V. The kit of any one of Paragraphs A-C and E-U, wherein the first transmembrane domain comprises a transmembrane region of CD8, CD28, CD3ζ, CD4 or 4-1BB
W. The kit of any one of Paragraphs A-C and E-V, wherein the reporter gene is located at the C-terminus of the first transmembrane domain or the glycosylphosphatidylinositol (GPI)-anchored polypeptide.
X. The kit of any one of Paragraphs A, C, and E-U, further comprising a spacer domain interspersed between the DOTA binding fragment and the transmembrane domain or the glycosylphosphatidylinositol (GPI)-anchored polypeptide.
Y. The kit of Paragraph X, wherein the spacer domain is a Fc domain or a polyhistidine tag.
Z. The kit of Paragraph Y, wherein the Fc domain comprises a Fc fragment of human IgG.
AA. The kit of Paragraph Z, wherein the Fc fragment of human IgG comprises the amino acid sequence of any one of SEQ ID NOs: 12-16.
AB. The kit of any one of Paragraphs A-C and E-AA, wherein the first transmembrane domain comprises an amino acid sequence that is at least 90% identical to an amino acid sequence of a transmembrane region of CD8, CD28, CD3ζ, or CD4 or 4-1BB ligand receptor.
AC. The kit of any one of Paragraphs A-C and E-AB, wherein the first and/or second transmembrane domain comprises the amino acid sequence of SEQ ID NO: 17.
AD. The kit of any one of Paragraphs A-C and E-AC, wherein the reporter gene is a fluorescent reporter gene, a chemiluminescent reporter gene, or a bioluminescent reporter gene.
AE. The kit of Paragraph AD, wherein the fluorescent reporter gene is GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa.
AF. The kit of Paragraph AE, wherein the bioluminescent reporter gene is Aequorin, firefly luciferase, Renilla luciferase, red luciferase, luxAB, or nanoluciferase.
AG. The kit of Paragraph AE, wherein the chemiluminescent reporter gene is β-galactosidase, horseradish peroxidase (HRP), or alkaline phosphatase.
AH. The kit of any one of Paragraphs A-AG, wherein the first fusion protein comprises the amino acid sequence of SEQ ID NO: 43, SEQ ID NO: 45, or SEQ ID NO: 46.
AI. The kit of any one of Paragraphs C and E-AH, wherein the first fusion protein does not include an internal ribosome entry site (IRES), or a 2A self-cleaving peptide.
AJ. The kit of any one of Paragraphs A-AI, further comprising streptavidin coated beads and optionally instructions for determining biotin-streptavidin binding activity of the biotinylated DOTA-based hapten.
AK. The kit of any one of Paragraphs A-AJ, wherein the first and/or second recombinant nucleic acid sequence is operably linked to an expression control sequence.
AL. The kit of Paragraph AK, wherein the expression control sequence is an inducible promoter or a constitutive promoter.
AM. The kit of any one of Paragraphs A-AL, wherein the biotinylated DOTA-based hapten is of Formula I

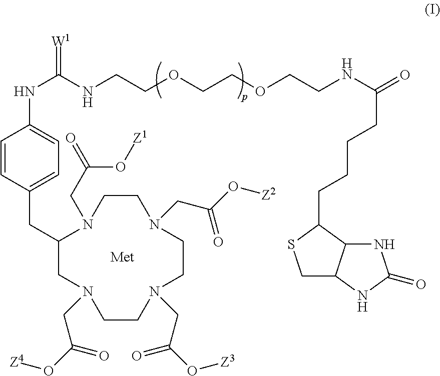

or a pharmaceutically acceptable salt thereof, wherein
   Met is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
   W¹ is S or O;
   Z¹, Z², Z³, and Z⁴ are each independently a lone pair of electrons (i.e., providing an oxygen anion) or H; and
   p is 1, 2, 3, 4, 5, 6, 7, 8, 9, 10, 11, 12, 13, 14, 15, 16, 17, 18, 19, 20, 21, or 22.
AN. The kit of Paragraph AM, wherein the biotinylated DOTA-based hapten of Formula I is of Formula IA

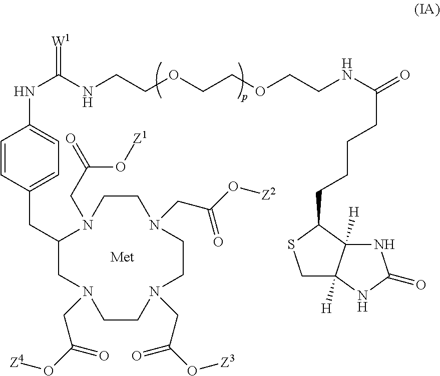

or a pharmaceutically acceptable salt thereof.
AO. The kit of any one of Paragraphs A-AN, wherein the DOTA-bearing bischelate is of Formula II

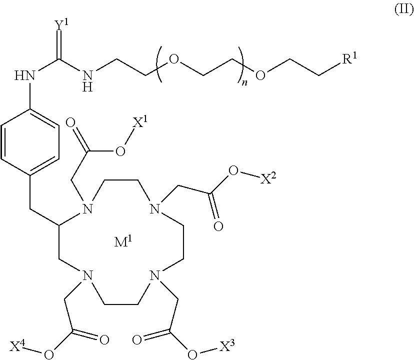

or a pharmaceutically acceptable salt thereof, wherein
M¹ is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
R¹ is

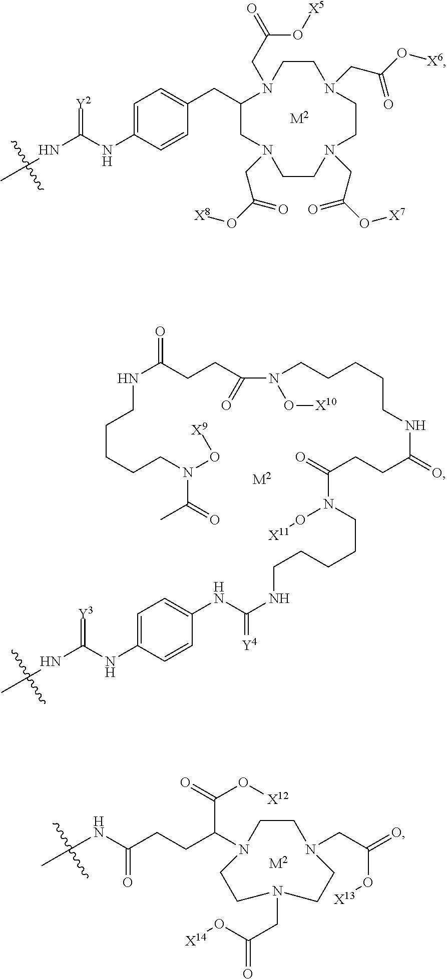

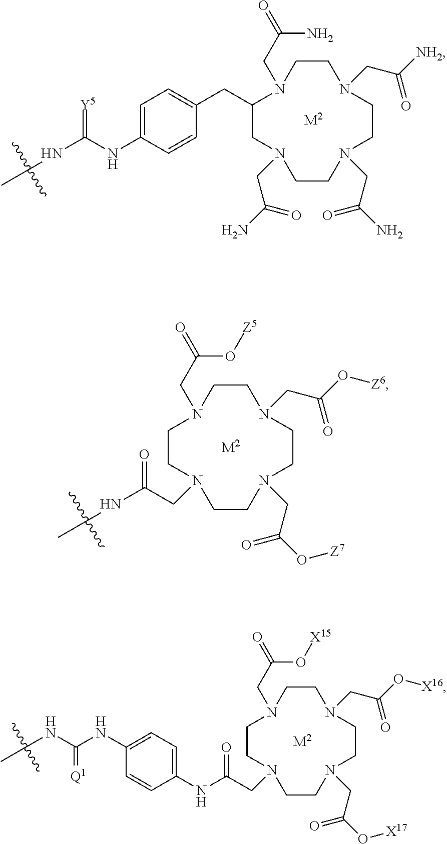

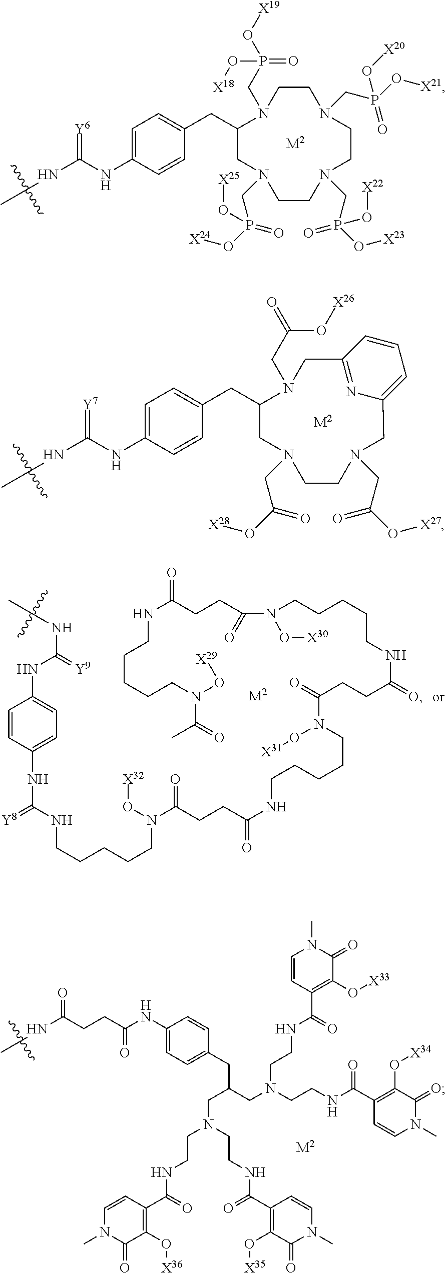

M2 is independently at each occurrence a radionuclide cation chelated by the R¹ group;
   X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², X²³, X²⁴, X²⁵, X²⁶, X²⁷, X²⁸, X²⁹, X³⁰, X³¹, X³², X³³, X³⁴, X³⁵, and X³⁶ are each independently a lone pair of electrons (i.e., providing an oxygen anion) or H;
   Z⁵, Z⁶, and Z⁷ are each independently a lone pair of electrons (i.e., providing an oxygen anion) or H;
   Y², Y³, Y⁴, Y⁵, Y⁶, Y⁷, Y⁸, and Y⁹ are each independently S or O;
   Q¹ is S or O; and
   n is 1, 2, 3, 4, 5, 6, 7, 8, 9, 10, 11, 12, 13, 14, 15, 16, 17, 18, 19, 20, 21, or 22.
AP. The kit of Paragraph AO, wherein M² is an alpha particle-emitting isotope, a beta particle-emitting isotope, an Auger-emitter, or a combination of any two or more thereof.
AQ. The kit of Paragraph AO or AP, wherein M² is ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, or ²⁵⁵Fm.
AR. The kit of any one of Paragraphs AO-AQ, wherein M² is ⁸⁶Y, ⁹⁰Y, ⁸⁹Sr, ¹⁶⁵Dy, ¹⁸⁶Re, ¹⁸⁸Re, ¹⁷⁷Lu, or ⁶⁷Cu.
AS. The kit of any one of Paragraphs AO-AQ, wherein M² is ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, or ²⁰³Pb.
AT. The kit of any one of Paragraphs AO-AQ, wherein M2 is ⁸⁹Zr, 86 Ga, ²¹²Pb, ²²⁷Th, or ⁶⁴Cu.
AU. The kit of any one of Paragraphs AO-AQ, wherein M² is ⁸⁹Zr, ²⁰³Pb, ²²⁷Th, or ⁶⁴Cu.
AV. The kit of any one of Paragraphs AO-AU, wherein M¹ is a chelated ¹⁷⁵Lu³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺.
AW. The kit of any one of Paragraphs AO-AU, wherein M¹ is a chelated ¹⁷⁵Lu³⁺, ¹¹³In³⁺, ¹¹⁵In³⁺, ¹³⁹La³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺.
AX. A fusion protein comprising a DOTA binding fragment fused to a first transmembrane domain, and a reporter gene, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively, and
   optionally wherein the fusion protein further comprises a receptor that binds to a target antigen.
AY. A fusion protein comprising a DOTA binding fragment fused to a glycosylphosphatidylinositol (GPI)-anchored polypeptide, and a receptor that binds to a target antigen, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively.
AZ. The fusion protein of Paragraph AX or Paragraph AY, wherein the receptor is a T cell receptor, a native cell receptor, a non-native cell receptor, or a chimeric antigen receptor, and/or wherein the target antigen is a tumor antigen.
BA. The fusion protein of Paragraph AZ, wherein the chimeric antigen receptor comprises (i) an extracellular antigen binding domain; (ii) a second transmembrane domain; and (iii) an intracellular domain, and optionally wherein the extracellular antigen binding domain binds to the target antigen.
BB. The fusion protein of Paragraph AZ or Paragraph BA, further comprising a self-cleaving peptide located between the DOTA binding fragment and the chimeric antigen receptor.
BC. A recombinant nucleic acid sequence encoding the fusion protein of any one of Paragraphs AX-BB.
BD. An expression vector comprising the recombinant nucleic acid sequence of Paragraph BC.
BE. A recombinant immune cell comprising the expression vector of Paragraph BD.
BF. The recombinant immune cell of Paragraph BE, wherein the recombinant immune cell is a T cell, a B cell, a tumor infiltrating lymphocyte, or a natural killer (NK) cell.
BG. The recombinant immune cell of Paragraph BE or Paragraph BF, wherein the recombinant nucleic acid sequence is operably linked to an expression control sequence that is heterologous or native to the recombinant immune cell.
BH. The recombinant immune cell of any one of Paragraphs BE-BG, wherein the recombinant immune cell is derived from an autologous donor or an allogenic donor.
BI. A method for determining the number of DOTA binding sites in a transduced immune cell comprising contacting the recombinant immune cell of any one Paragraphs BE-BH with a biotinylated DOTA-based hapten, and determining the binding activity of the biotinylated DOTA-based hapten to the recombinant immune cell, wherein the recombinant immune cell exhibits cell surface expression of the fusion protein of any one of Paragraphs AX-BB.
BJ. The method of Paragraph BI, wherein the binding activity of the biotinylated DOTA-based hapten is determined via saturation binding assays or competition binding assays.
BK. The method of Paragraph BI or Paragraph BJ, further comprising determining a mean equilibrium dissociation constant (Kd) of the biotinylated DOTA-based hapten and a mean Bmax (sites/cell) for the fusion protein.
BL. A method for tracking recombinant immune cells in a subject in vivo comprising
   (a) administering to the subject an effective amount of the recombinant immune cell of any one of Paragraphs BE-BH, wherein the recombinant immune cell is configured to localize to a tissue expressing the target antigen recognized by the recombinant immune cell;
   (b) administering to the subject an effective amount of a DOTA-bearing bischelate, wherein the DOTA-bearing bischelate is configured to bind to the fusion protein expressed by the recombinant immune cell and comprises a radionuclide; and
   (c) determining the biodistribution of the recombinant immune cells in the subject by detecting radioactive levels emitted by the DOTA-bearing bischelate that are higher than a reference value.
BM. A method for tracking recombinant immune cells in a subject in vivo comprising
   (a) administering to the subject an effective amount of a complex comprising the recombinant immune cell of any one of Paragraphs BE-BH and a DOTA-bearing bischelate comprising a radionuclide, wherein the complex is configured to localize to a tissue expressing the target antigen recognized by the recombinant immune cell; and
   (b) determining the biodistribution of recombinant immune cells in the subject by detecting radioactive levels emitted by the DOTA-bearing bischelate that are higher than a reference value.
BN. A method for monitoring viability of recombinant immune cells in a subject comprising:
   (a) administering to the subject an effective amount of the recombinant immune cell of any one of Paragraphs BE-BH, wherein the recombinant immune cell is configured to localize to a tissue expressing the target antigen recognized by the recombinant immune cell;
   (b) administering to the subject an effective amount of a DOTA-bearing bischelate, wherein the DOTA-bearing bischelate is configured to bind to the fusion protein expressed by the recombinant immune cell and comprises a radionuclide;
   (c) detecting radioactive levels emitted by the DOTA-bearing bischelate that are higher than a reference value at a first time point;
   (d) detecting radioactive levels emitted by the DOTA-bearing bischelate that are higher than a reference value at a second time point; and
   (e) determining that the recombinant immune cells in the subject are viable when the radioactive levels emitted by the DOTA-bearing bischelate at the second time point are comparable to that observed at the first time point.
BO. The method of Paragraph BN, further comprising administering to the subject a second effective amount of the DOTA-bearing bischelate prior to step (d).
BP. A method for monitoring viability of recombinant immune cells in a subject comprising:
   (a) administering to the subject an effective amount of a complex comprising the recombinant immune cell of any one of Paragraphs BE-BH and a DOTA-bearing bischelate comprising a radionuclide, wherein the complex is configured to localize to a tissue expressing the target antigen recognized by the recombinant immune cell;
   (b) detecting radioactive levels emitted by the DOTA-bearing bischelate that are higher than a reference value at a first time point;
   (c) detecting radioactive levels emitted by the DOTA-bearing bischelate that are higher than a reference value at a second time point; and
   (d) determining that the recombinant immune cells in the subject are viable when the radioactive levels emitted by the DOTA-bearing bischelate at the second time point are comparable to that observed at the first time point.
BQ. A method for monitoring expansion of recombinant immune cells in a subject comprising:
   (a) administering to the subject an effective amount of the recombinant immune cell of any one of Paragraphs BE-BH, wherein the recombinant immune cell is configured to localize to a tissue expressing the target antigen recognized by the recombinant immune cell;
   (b) administering to the subject a first effective amount of a DOTA-bearing bischelate, wherein the DOTA-bearing bischelate is configured to bind to the fusion protein expressed by the recombinant immune cell and comprises a radionuclide;
   (c) detecting radioactive levels emitted by the DOTA-bearing bischelate that are higher than a reference value at a first time point;
   (d) administering to the subject a second effective amount of the DOTA-bearing bischelate after step (c);
   (e) detecting radioactive levels emitted by the DOTA-bearing bischelate that are higher than a reference value at a second time point; and
   (f) determining that the recombinant immune cells in the subject have expanded when the radioactive levels emitted by the DOTA-bearing bischelate at the second time point are higher relative to that observed at the first time point.
BR. The method of any one of Paragraphs BL-BQ, wherein the radioactive levels emitted by the complex or the DOTA-bearing bischelate are detected using positron emission tomography or single photon emission computed tomography.
BS. The method of any one of Paragraphs BL-BR, wherein the DOTA-bearing bischelate is of Formula II

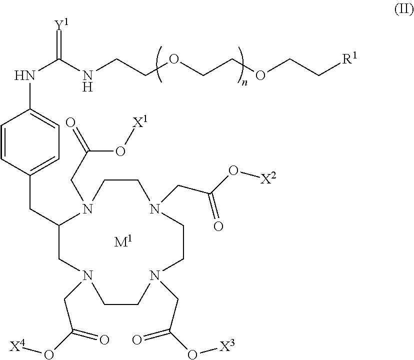

or a pharmaceutically acceptable salt thereof, wherein
M¹ is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In¹³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
R¹ is

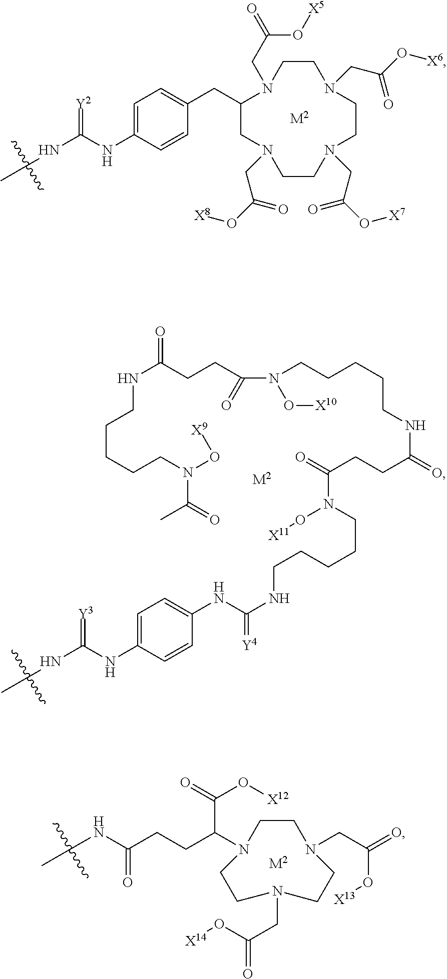

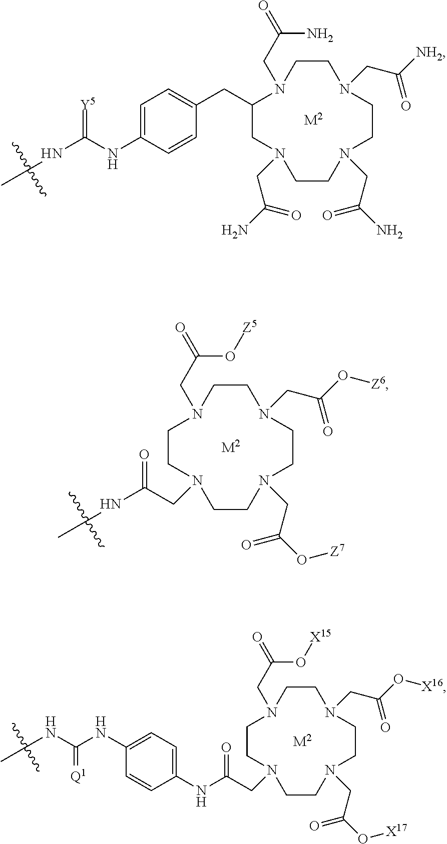

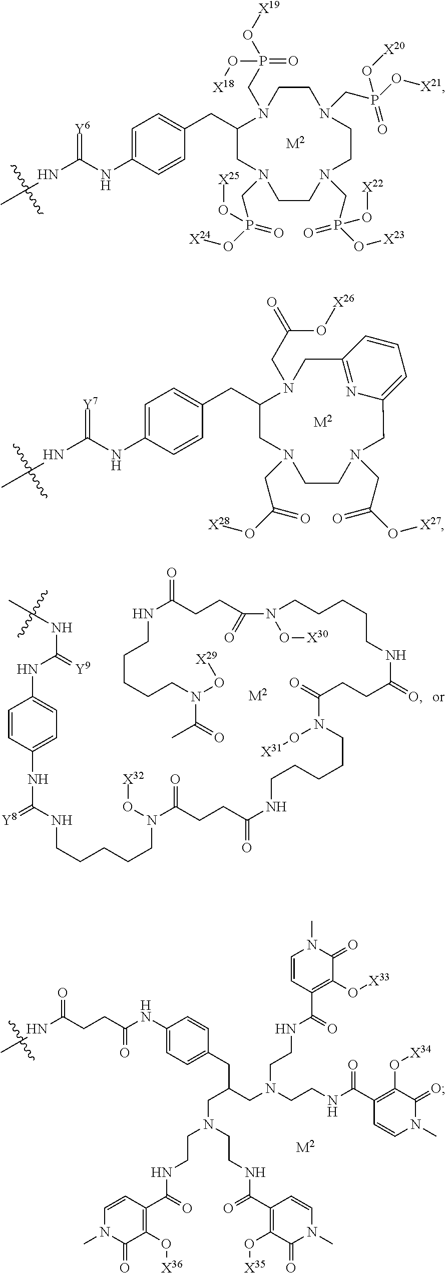

M² is independently at each occurrence a radionuclide cation chelated by the R¹ group;
X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², X²³, X²⁴, X²⁵, X²⁶, X²⁷, X²⁸, X²⁹, X³⁰, X³¹, X³², X³³, X³⁴, X³⁵, and X³⁶ are each independently a lone pair of electrons (i.e., providing an oxygen anion) or H;
Z⁵, Z⁶, and Z⁷ are each independently a lone pair of electrons (i.e., providing an oxygen anion) or H
Y², Y³, Y⁴, Y⁵, Y⁶, Y⁷, Y⁸, and Y⁹ are each independently S or O;
Q¹ is S or O; and
n is 1, 2, 3, 4, 5, 6, 7, 8, 9, 10, 11, 12, 13, 14, 15, 16, 17, 18, 19, 20, 21, or 22.
BT. The method of any one of Paragraphs BL-BS, wherein the radionuclide is an alpha particle-emitting isotope, a beta particle-emitting isotope, an Auger-emitter, or a combination of any two or more thereof (e.g., M² is an alpha particle-emitting isotope, a beta particle-emitting isotope, an Auger-emitter, or a combination of any two or more thereof).
BU. The method of any one of Paragraphs BL-BT, wherein the radionuclide is ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu (e.g. M² is ²¹³Bi, ²¹¹At ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu).
BV. The method of any one of Paragraphs BL-BU, wherein the subject is diagnosed with or is suffering from a cancer selected from the group consisting of a carcinoma, a sarcoma, a melanoma, a hematopoietic cancer, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Other embodiments are set forth in the following claims.

1. A kit comprising
(I)
(a) a first expression vector comprising a first recombinant nucleic acid sequence that encodes a first fusion protein comprising a DOTA binding fragment fused to a first transmembrane domain, and a reporter gene, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively, optionally wherein the first fusion protein further comprises a receptor that binds to a target antigen; and
(b) a biotinylated DOTA-based hapten; and
(c) a DOTA-bearing bischelate, optionally wherein the kit further comprises a second expression vector comprising a second recombinant nucleic acid sequence that encodes a second fusion protein comprising a receptor that binds to a target antigen; or
(II)
(a) a first recombinant nucleic acid sequence encoding a first fusion protein comprising a DOTA binding fragment fused to a glycosylphosphatidylinositol (GPI)-anchored polypeptide, and a receptor that binds to a target antigen, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively; and
(b) a biotinylated DOTA-based hapten; and
(c) a DOTA-bearing bischelate.

2. (canceled)

3. (canceled)

4. (canceled)

5. The kit of claim 1, wherein the receptor is a T cell receptor, a native cell receptor, a non-native cell receptor, or a chimeric antigen receptor; or
wherein the first fusion protein further comprises a spacer domain interspersed between the DOTA binding fragment and the first transmembrane domain or the glycosylphosphatidylinositol (GPI)-anchored polypeptide, optionally wherein the spacer domain is a Fc domain or a polyhistidine tag, optionally wherein the Fc domain comprises a Fc fragment of human IgG or comprises the amino acid sequence of any one of SEQ ID NOs: 12-16.

6. The kit of claim 5, wherein the chimeric antigen receptor comprises
(i) an extracellular antigen binding domain that binds to the target antigen, optionally wherein the target antigen is a tumor antigen;
(ii) a second transmembrane domain, optionally wherein the second transmembrane domain comprises an amino acid sequence that is at least 90% identical to an amino acid sequence of a transmembrane region of CD8, CD28, CD3ζ, CD4, or 4-1BB ligand receptor; and
(iii) an intracellular domain, optionally wherein the intracellular domain comprises one or more costimulatory domains, optionally wherein the one or more costimulatory domains are selected from a CD28 costimulatory domain, a CD3ζ-chain, a 4-1BBL costimulatory domain, or any combination thereof;
optionally wherein the tumor antigen is selected from the group consisting of 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1.

7. (canceled)

8. (canceled)

9. (canceled)

10. The kit of claim 6, wherein the extracellular antigen binding domain comprises
a single chain variable fragment (scFv), optionally wherein the scFv is a human scFv; or
a CD19 scFv of SEQ ID NO: 27 or SEQ ID NO: 28; or
a CD19 scFv having at least 80%, 85%, 90%, 95%, 96%, 97%, 98%, or 99% sequence identity to SEQ ID NO: 27 or SEQ ID NO: 28; or
a signal peptide that is covalently joined to the N-terminus of the extracellular antigen binding domain.

11. (canceled)

12. (canceled)

13. (canceled)

14. (canceled)

15. (canceled)

16. (canceled)

17. (canceled)

18. The kit of claim 1, wherein the DOTA binding fragment is located at the N-terminus of the first transmembrane domain or the glycosylphosphatidylinositol (GPI)-anchored polypeptide; or
wherein the first and/or second fusion protein further comprises an endoplasmic reticulum signal sequence; or
wherein the sequence of an intra-peptide linker between the V_{H} and the V_{L} in the DOTA binding fragment is any one of SEQ ID NOs: 9-11; or
the V_{H} is located at the N-terminus or the C-terminus of the V_{L}; or
wherein the first transmembrane domain comprises a transmembrane region of CD8, CD28, CD3ζ, CD4 or 4-1BB; or
wherein the reporter gene is located at the C-terminus of the first transmembrane domain; or
wherein the first transmembrane domain comprises an amino acid sequence that is at least 90% identical to an amino acid sequence of a transmembrane region of CD8, CD28, CD3ζ, or CD4 or 4-1BB ligand receptor; or
wherein the first and/or second transmembrane domain comprises the amino acid sequence of SEQ ID NO: 17; or
wherein the first fusion protein comprises the amino acid sequence of SEQ ID NO: 43, SEQ ID NO: 45, or SEQ ID NO: 46; or
wherein the first fusion protein does not include an internal ribosome entry site (IRES), or a 2A self-cleaving peptide; or
wherein the first and/or second recombinant nucleic acid sequence is operably linked to an expression control sequence, optionally wherein the expression control sequence is an inducible promoter or a constitutive promoter; or
wherein the reporter gene is a fluorescent reporter gene, a chemiluminescent reporter gene, or a bioluminescent reporter gene.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The kit of claim 18, wherein the fluorescent reporter gene is GFP, YFP, CFP, RFP, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa; or
wherein the bioluminescent reporter gene is Aequorin, firefly luciferase, Renilla luciferase, red luciferase, luxAB, or nanoluciferase; or
wherein the chemiluminescent reporter gene is β-galactosidase, horseradish peroxidase (HRP), or alkaline phosphatase.

32. (canceled)

33. (canceled)

34. (canceled)

35. (canceled)

36. (canceled)

37. (canceled)

38. (canceled)

39. The kit of claim 1, wherein the biotinylated DOTA-based hapten is of Formula I 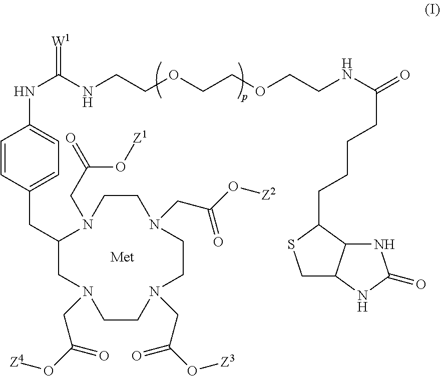
or a pharmaceutically acceptable salt thereof, wherein
Met is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
W¹ is S or O;
Z¹, Z², Z³, and Z⁴ are each independently a lone pair of electrons (i.e., providing an oxygen anion) or H; and
p is 1, 2, 3, 4, 5, 6, 7, 8, 9, 10, 11, 12, 13, 14, 15, 16, 17, 18, 19, 20, 21, or 22,
optionally wherein the biotinylated DOTA-based hapten of Formula I is of Formula IA 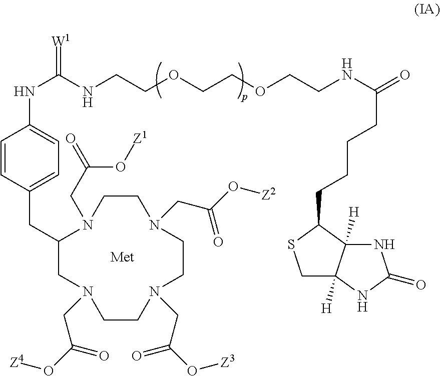
or a pharmaceutically acceptable salt thereof, and/or wherein the kit further comprises streptavidin coated beads and optionally instructions for determining biotin-streptavidin binding activity of the biotinylated DOTA-based hapten.

40. (canceled)

41. The kit of claim 1, wherein the DOTA-bearing bischelate is of Formula II 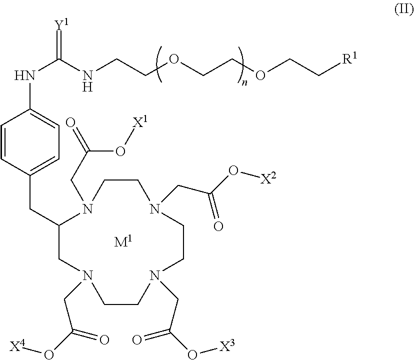
or a pharmaceutically acceptable salt thereof, wherein
M¹ is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In¹³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
R¹ is 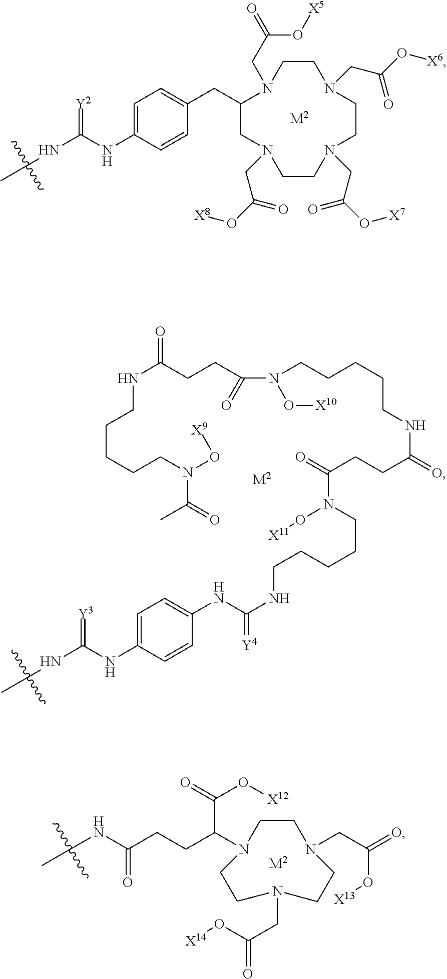 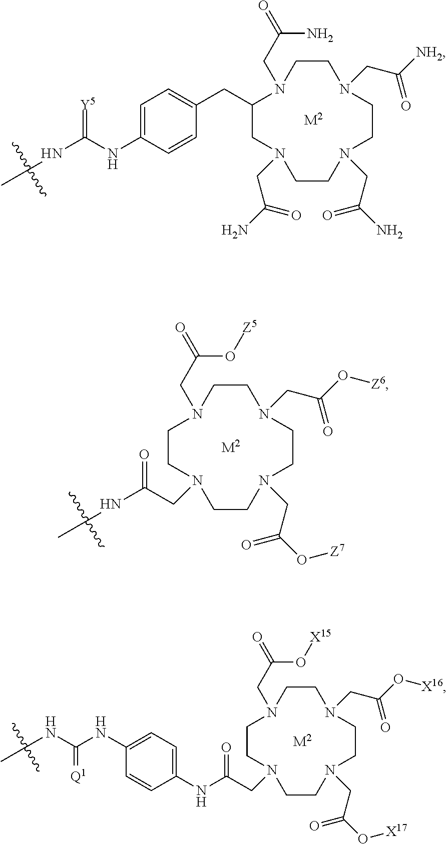 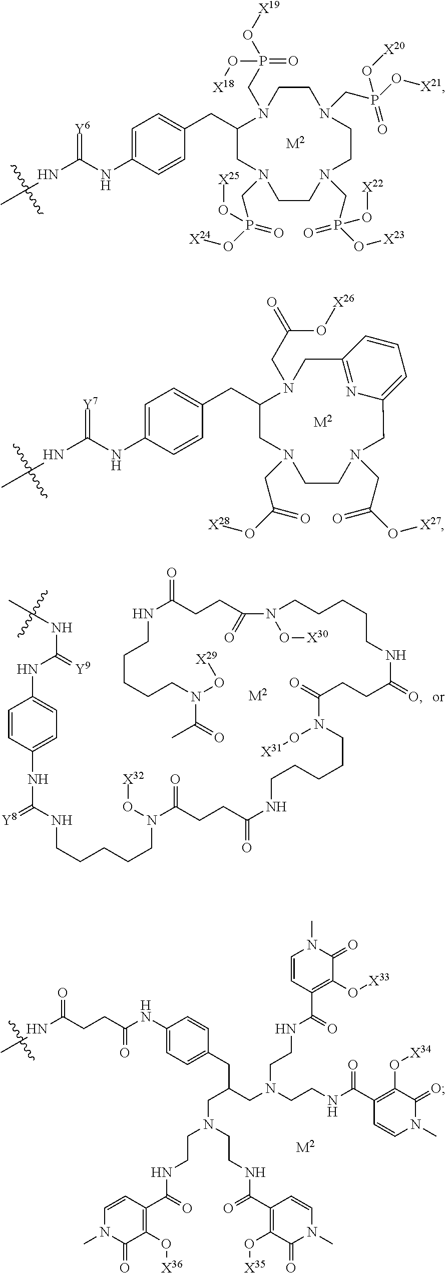
M² is independently at each occurrence a radionuclide cation chelated by the R¹ group;
X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², X²³, X²⁴, X²⁵, X²⁶, X²⁷, X²⁸, X²⁹, X³⁰, X³¹, X³², X³³, X³⁴, X³⁵, and X³⁶ are each independently a lone pair of electrons (i.e., providing an oxygen anion) or H;
Z⁵, Z⁶, and Z⁷ are each independently a lone pair of electrons (i.e., providing an oxygen anion) or H;
Y¹, Y², Y³, Y⁴, Y⁵, Y⁶, Y⁷, Y⁸, and Y⁹ are each independently S or O;
Q¹ is S or O; and
n is 1, 2, 3, 4, 5, 6, 7, 8, 9, 10, 11, 12, 13, 14, 15, 16, 17, 18, 19, 20, 21, or 22.

42. The kit of claim 41, wherein M² is an alpha particle-emitting isotope, a beta particle-emitting isotope, an Auger-emitter, or a combination of any two or more thereof; or
wherein M² is ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, or ²⁵⁵Fm; or
wherein M² is ⁸⁶Y, ⁹⁰Y, ⁸⁹Sr, ¹⁶⁵Dy, ¹⁸⁶Re, ¹⁸⁸Re, ¹⁷⁷Lu, or ⁶⁷Cu, or
wherein M² is ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, or ²⁰³Pb; or
wherein M² is ⁸⁹Zr, ⁶⁸Ga, ²¹²Pb, ²²⁷Th, or ⁶⁴Cu; or
wherein M² is ⁸⁹Zr, ²⁰³Pb, ²²⁷Th, or ⁶⁴Cu; or
wherein M¹ is a chelated ¹⁷⁵Lu³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺; or
wherein M¹ is a chelated ¹⁷⁵Lu³⁺, ¹¹³In³⁺, ¹¹⁵In³⁺, ¹³⁹La³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺.

43. (canceled)

44. (canceled)

45. (canceled)

46. (canceled)

47. (canceled)

48. (canceled)

49. (canceled)

50. A fusion protein comprising
(I) a DOTA binding fragment fused to a first transmembrane domain, and a reporter gene, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively, and optionally wherein the fusion protein further comprises a receptor that binds to a target antigen; or
(II) a DOTA binding fragment fused to a glycosylphosphatidylinositol (GPI)-anchored polypeptide, and a receptor that binds to a target antigen, wherein the DOTA binding fragment comprises a heavy chain immunoglobulin variable domain (V_{H}) and a light chain variable immunoglobulin domain (V_{L}) comprising the amino acid sequence of SEQ ID NO: 1, and SEQ ID NO: 5, respectively.

51. (canceled)

52. The fusion protein of claim 50, wherein the receptor is a T cell receptor, a native cell receptor, a non-native cell receptor, or a chimeric antigen receptor, and/or wherein the target antigen is a tumor antigen, optionally wherein
the chimeric antigen receptor comprises (i) an extracellular antigen binding domain; (ii) a second transmembrane domain; and (iii) an intracellular domain, and optionally wherein the extracellular antigen binding domain binds to the target antigen; or the fusion protein further comprises a self-cleaving peptide located between the DOTA binding fragment and the chimeric antigen receptor.

53. (canceled)

54. (canceled)

55. A recombinant nucleic acid sequence encoding the fusion protein of claim 50.

56. An expression vector comprising the recombinant nucleic acid sequence of claim 55.

57. A recombinant immune cell comprising the expression vector of claim 56, optionally wherein
the recombinant immune cell is a T cell, a B cell, a tumor infiltrating lymphocyte, or a natural killer (NK) cell; or
wherein the recombinant immune cell is derived from an autologous donor or an allogenic donor; or
wherein the recombinant nucleic acid sequence is operably linked to an expression control sequence that is heterologous or native to the recombinant immune cell.

58. (canceled)

59. (canceled)

60. (canceled)

61. A method for determining the number of DOTA binding sites in a transduced immune cell comprising contacting the recombinant immune cell of claim 57 with a biotinylated DOTA-based hapten, and determining the binding activity of the biotinylated DOTA-based hapten to the recombinant immune cell, wherein the recombinant immune cell exhibits cell surface expression of the fusion protein.

62. The method of claim 61, wherein the binding activity of the biotinylated DOTA-based hapten is determined via saturation binding assays or competition binding assays or wherein the method further comprises determining a mean equilibrium dissociation constant (Kd) of the biotinylated DOTA-based hapten and a mean Bₘₐₓ (sites/cell) for the fusion protein.

63. (canceled)

64. A method for tracking recombinant immune cells in a subject in vivo comprising
(I)
(a) administering to the subject an effective amount of a recombinant immune cell, wherein the recombinant immune cell is configured to localize to a tissue expressing the target antigen recognized by the recombinant immune cell;
(b) administering to the subject an effective amount of a DOTA-bearing bischelate, wherein the DOTA-bearing bischelate is configured to bind to the fusion protein expressed by the recombinant immune cell and comprises a radionuclide; and
(c) determining the biodistribution of the recombinant immune cells in the subject by detecting radioactive levels emitted by the DOTA-bearing bischelate that are higher than a reference value; or
(II)
(a) administering to the subject an effective amount of a complex comprising a recombinant immune cell and a DOTA-bearing bischelate comprising a radionuclide, wherein the complex is configured to localize to a tissue expressing the target antigen recognized by the recombinant immune cell; and
(b) determining the biodistribution of recombinant immune cells in the subject by detecting radioactive levels emitted by the DOTA-bearing bischelate that are higher than a reference value,
wherein the recombinant immune cell is the recombinant immune cell of claim 57.

65. (canceled)

66. A method for monitoring viability of recombinant immune cells in a subject comprising:
(I)
(a) administering to the subject an effective amount of a recombinant immune cell, wherein the recombinant immune cell is configured to localize to a tissue expressing the target antigen recognized by the recombinant immune cell;
(b) administering to the subject an effective amount of a DOTA-bearing bischelate, wherein the DOTA-bearing bischelate is configured to bind to the fusion protein expressed by the recombinant immune cell and comprises a radionuclide;
(c) detecting radioactive levels emitted by the DOTA-bearing bischelate that are higher than a reference value at a first time point;
(d) detecting radioactive levels emitted by the DOTA-bearing bischelate that are higher than a reference value at a second time point; and
(e) determining that the recombinant immune cells in the subject are viable when the radioactive levels emitted by the DOTA-bearing bischelate at the second time point are comparable to that observed at the first time point,
optionally wherein the method further comprises administering to the subject a second effective amount of the DOTA-bearing bischelate prior to step (d); or
(II)
(a) administering to the subject an effective amount of a complex comprising a recombinant immune cell and a DOTA-bearing bischelate comprising a radionuclide, wherein the complex is configured to localize to a tissue expressing the target antigen recognized by the recombinant immune cell;
(b) detecting radioactive levels emitted by the DOTA-bearing bischelate that are higher than a reference value at a first time point;
(c) detecting radioactive levels emitted by the DOTA-bearing bischelate that are higher than a reference value at a second time point; and
(d) determining that the recombinant immune cells in the subject are viable when the radioactive levels emitted by the DOTA-bearing bischelate at the second time point are comparable to that observed at the first time point,
wherein the recombinant immune cell is the recombinant immune cell of claim 57.

67. (canceled)

68. (canceled)

69. A method for monitoring expansion of recombinant immune cells in a subject comprising:
(a) administering to the subject an effective amount of the recombinant immune cell of claim 57, wherein the recombinant immune cell is configured to localize to a tissue expressing the target antigen recognized by the recombinant immune cell;
(b) administering to the subject a first effective amount of a DOTA-bearing bischelate, wherein the DOTA-bearing bischelate is configured to bind to the fusion protein expressed by the recombinant immune cell and comprises a radionuclide;
(c) detecting radioactive levels emitted by the DOTA-bearing bischelate that are higher than a reference value at a first time point;
(d) administering to the subject a second effective amount of the DOTA-bearing bischelate after step (c);
(e) detecting radioactive levels emitted by the DOTA-bearing bischelate that are higher than a reference value at a second time point; and
(f) determining that the recombinant immune cells in the subject have expanded when the radioactive levels emitted by the DOTA-bearing bischelate at the second time point are higher relative to that observed at the first time point.

70. The method of claim 64, wherein the radioactive levels emitted by the complex or the DOTA-bearing bischelate are detected using positron emission tomography or single photon emission computed tomography; or wherein the DOTA-bearing bischelate is of Formula II 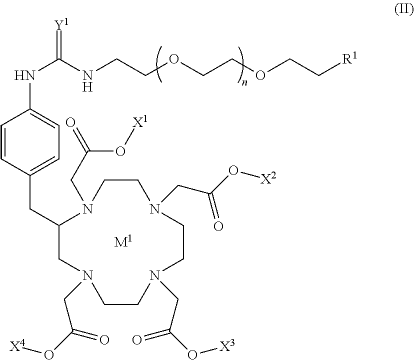
or a pharmaceutically acceptable salt thereof, wherein
M¹ is a chelated ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
R¹ is 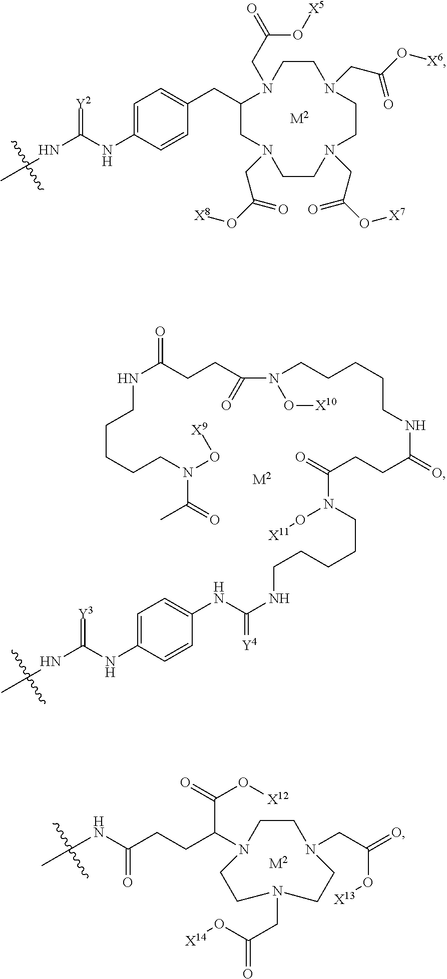 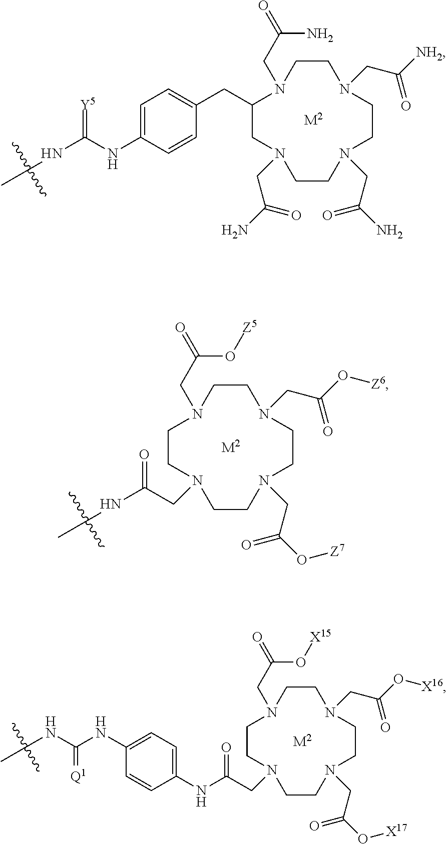 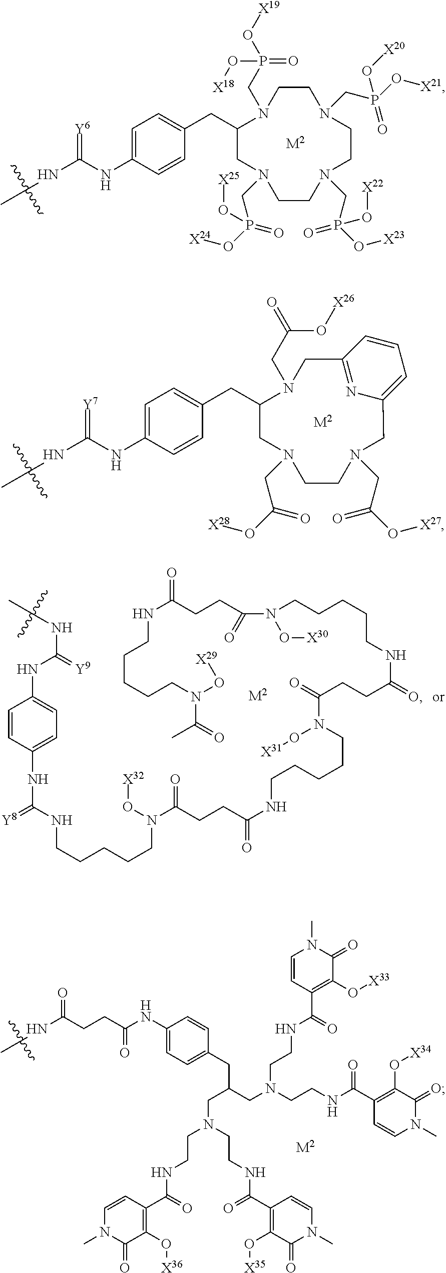
M² is independently at each occurrence a radionuclide cation chelated by the R¹ group;
X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², X²³, X²⁴, X²⁵, X²⁶, X²⁷, X²⁸, X²⁹, X³⁰, X³¹, X³², X³³, X³⁴, X³⁵, and X³⁶ are each independently a lone pair of electrons (i.e., providing an oxygen anion) or H;
Z⁵, Z⁶, and Z⁷ are each independently a lone pair of electrons (i.e., providing an oxygen anion) or H;
Y¹, Y², Y³, Y⁴, Y⁵, Y⁶, Y⁷, Y⁸ and Y⁹ are each independently S or O;
Q¹ is S or O; and
n is 1, 2, 3, 4, 5, 6, 7, 8, 9, 10, 11, 12, 13, 14, 15, 16, 17, 18, 19, 20, 21, or 22; or
wherein the radionuclide is an alpha particle-emitting isotope, a beta particle-emitting isotope, an Auger-emitter, or a combination of any two or more thereof (e.g., M² is an alpha particle-emitting isotope, a beta particle-emitting isotope, an Auger-emitter, or a combination of any two or more thereof); or
wherein the radionuclide is ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, 58Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu (e.g., M² is ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, ²⁵⁵Fm, ⁸⁶Y, ⁹⁰Y, ⁸⁹Sr, ¹⁶⁵Dy, ¹⁸⁶Re, ¹⁸⁸Re, ¹⁷⁷Lu, ⁶⁷Cu, ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁶⁸Ga, ²²⁷Th, or ⁶⁴Cu); or
wherein the subject is diagnosed with or is suffering from a cancer selected from the group consisting of a carcinoma, a sarcoma, a melanoma, a hematopoietic cancer, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

71. (canceled)

72. (canceled)

73. (canceled)

74. (canceled)
